(19) United States
(12) Patent Application Publication
(10) Pub. No.: US 2023/0167142 A1
(43) Pub. Date: Jun. 1, 2023

(54) METHODS FOR THE MONO-AMIDATION OF PHOSPHATES AND PHOSPHONATES

(71) Applicant: Board of Regents, The University of Texas System, Austin, TX (US)

(72) Inventors: Victoria YAN, Hillsborough, CA (US); Cong-Dat PHAM, Houston, TX (US); Florian MULLER, Houston, TX (US)

(21) Appl. No.: 17/936,970

(22) Filed: Sep. 30, 2022

Related U.S. Application Data
(63) Continuation of application No. PCT/US2021/025399, filed on Apr. 1, 2021.
(60) Provisional application No. 63/004,063, filed on Apr. 2, 2020.
(60) Provisional application No. 63/108,099, filed on Oct. 30, 2020.

(51) Int. Cl. C07F 9/59; C07F 9/6558; C07F 9/24; C07F 9/58; C07H 19/20; C07F 9/6561; A61K 45/06; A61P 35/00

(52) U.S. Cl. CPC C07F 9/59; C07F 9/65583; C07F 9/24; C07F 9/58; C07H 19/20; C07F 9/65616; A61K 45/06; A61P 35/00

(57) ABSTRACT

Provided is a method for the preparation of mono-amidated phosphates or phosphonates as well as mono-amidated phosphates and phosphonates prepared by such method.

This application is a bypass continuation of International Application No. PCT/US2021/025399, filed Apr. 1, 2021, which claims priority to U.S. Application No. 63/004,063, filed Apr. 2, 2020, and U.S. Application No. 63/108,099, filed Oct. 30, 2020, the contents of which are incorporated herein by reference for all purposes.

Phosphoramidates and phosphonoamidates are structurally intriguing chemical moieties with high biological and therapeutic relevance. Inclusion of a phosphoramidate moiety has become an increasingly attractive pro-drug strategy for anionic phosphate- or phosphonate-containing drugs. While the development of phosphoramidate and phosphonoamidate prodrugs has been primarily spearheaded by the antiviral nucleotide field, this approach is emerging as an appealing strategy for the delivery of novel phosphonate-containing cancer therapeutics. Especially amidst the emergence of precision medicine, the efficient preparation of phosphoramidates proves to be a therapeutically significant endeavor.

Historically, phosphoramidates have been prepared using harsh reaction conditions involving toxic reagents. Alternative approaches that involve initial assembly of a phosphoramidate fragment, followed by N-methylimidazole (NMI) or t-butylmagnesium chloride are highly inconsistent and poor-yielding, while peptide coupling strategies using N—N′-dicyclohexylcarbodiimide (DCC) or 1-ethyl-3-(3-dimethylaminopropyl)carbodiimide (EDC) suffer from the difficulty of isolating the water-soluble mono-phosphoramidate from the water-soluble urea byproducts. In general, the development of phosphoramidate-containing molecules has been offset by the high variability in reaction efficiency and poor purification techniques to preserve the integrity of the acid-sensitive phosphoramidate.

The Mitsunobu reaction is a highly versatile reaction that converts an alcohol into a number of functional groups such as an ester or an ether. In more niche instances, it can be used to generate sulfonamides, thioesters, and N-alkylamines. However, since its initial inception in 1967, the Mitsunobu reaction has not been applied to a phosphate or phosphonate and an amine to generate the corresponding phosphoramidate. There is a need for new methods to generate phosphoramidates. The present disclosure fulfills these and other needs, as evident in reference to the following disclosure.

### BRIEF DESCRIPTION OF THE DRAWINGS

FIGS. 1A, 1B, 1C, and 1D show cell proliferation assays for enolase inhibitors HEX (1A), FLM37 (1B), CDP18 (1C), and VCY32 (1D) against (i) ENO1-deleted (D423), (ii) ENO1-rescued (D423 ENO1), and (iii) ENO1-wildtype (LN319) glioma cells. Cells were incubated with pro-drug inhibitor for 5 days. Then, cells were fixed and stained with crystal violet and quantified spectroscopically. Numerical labels indicate concentration of enolase inhibitor in nM.

FIGS. 2A, 2B, and 2C show plots of relative cell density (vertical axis) against concentration (horizontal axis, nM) of enolase inhibitors FLM37 (2A), CDP18 (2B), and VCY32 (2C) for (i) ENO1-deleted (D423), (ii) ENO1-rescued (D423 ENO1), and (iii) ENO1-wildtype (LN319) glioma cells.

FIGS. 3A and 3B show cell proliferation assays for enolase inhibitors VCY33 (3A) and CDP22 (3B) against (i) ENO1-deleted (D423) and (ii) ENO1-wildtype (LN319) glioma cells. Numerical labels indicate concentration of enolase inhibitor in nM.

FIGS. 4A and 4B show plots of relative cell density (vertical axis) against concentration (horizontal axis, nM) of enolase inhibitors VCY33 (4A) and CDP22 (4B) for (i) ENO1-deleted glioma cells (D423) and (ii) ENO1-wildtype (LN319) glioma cells.

FIG. 5 shows strategies for employing benzylamine-based prodrugs to achieve enolase inhibition. The indicated compounds can be activated by (a) carboxyesterase activity, (b) E2 elimination, (c) thioesterase activity, or (d) nitroreductase activity, followed by (e) phosphoramidase activity.

FIG. 6 shows strategies for employing prodrugs based on aliphatic amines to achieve enolase inhibition. The indicated compounds can be activated by (a) carboxyesterase activity, (b) E2 elimination, (c) thioesterase activity, or (d) nitroreductase activity, followed by (e) phosphoramidase activity.

FIG. 7 shows strategies for employing prodrugs based on methylamine to achieve enolase inhibition. The indicated compounds can be activated by (a) carboxyesterase activity, (b) E2 elimination, (c) thioesterase activity, or (d) nitroreductase activity, followed by (e) phosphoramidase activity.

FIG. 8 shows strategies for employing prodrugs to achieve inhibition of viral polymerases. The indicated compounds can be activated by (a) carboxyesterase activity, (b) E2 elimination, (c) thioesterase activity, or (d) nitroreductase activity, followed by (e) phosphoramidase and (f) nucleotide kinase activity.

### BRIEF SUMMARY

Provided is a method for preparing a compound of Formula I

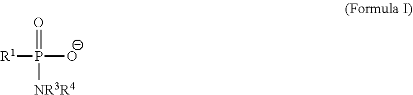

wherein

R¹ is chosen from —R² and —OR²;

R² is chosen from alkyl_{(C≤12)}, cycloalkyl_{(C≤12)}, heterocycloalkyl_{(C≤12)}, aryl_{(C≤12)}, and heteroaryl_{(C≤12)}, or a substituted version of any of these groups;

R³ is chosen from hydrogen, alkyl_{(C≤18)}, and substituted alkyl_{(C≤18)}; and

R⁴ is chosen from alkyl_{(C≤18)}, aryl_{(C≤18)}, aralkyl_{(C≤18)}, heteroaralkyl_{(C≤18)}, alkanediyl_{(C≤18)}-alkoxy_{(C≤18)}, cycloalkyl_{(C≤18)}, alkanediyl_{(C≤18)}-cycloalkyl_{(C≤18)}, or a substituted version of any of these groups; or

R³ and R⁴, together with the intervening nitrogen, combine to form a 5-7 membered heterocycloalkyl;

said method comprising:

treating a compound of Formula II

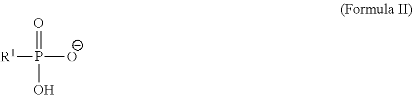

with a dialkylazodicarboxylate; a phosphine reagent; and an amine of Formula III

NHR³R⁴ (Formula III)
in an aprotic solvent to yield a compound of Formula I.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The terms “treating” “adding” “reacting” and “mixing” are used interchangeably to refer to contacting one reactant, reagent, solvent, catalyst, or a reactive group with another reactant, reagent, solvent, catalyst, or reactive group. Unless otherwise specified, reactants, reagents, solvents, catalysts, and reactive groups can be added individually, simultaneously, or separately, and/or can be added in any order. They can be added in the presence or absence of heat, and can optionally be added under an inert atmosphere (e.g., N₂ or Ar).

The term “therapeutically effective amount refers to the quantity of a specific substance, such as a disclosed agent, sufficient to achieve a desired therapeutic effect in a subject being treated. In some embodiments, a therapeutically effective amount is the amount necessary to inhibit an immunodeficiency virus replication or treat AIDS in a subject with an existing infection with the immunodeficiency virus.

The term “prevent,” “preventing” or “prevention” means prevention of the occurrence or onset of one or more symptoms associated with a particular disorder and does not necessarily mean the complete prevention of a disorder. For example, the term “prevent,” “preventing” and “prevention” refers to the administration of therapy on a prophylactic or preventative basis to an individual who may ultimately manifest at least one symptom of a disease or condition but who has not yet done so. Such individuals can be identified since risk factors that are known to correlate with the subsequent occurrence of the disease. Alternatively, prevention therapy can be administered without prior identification of a risk factor, as a prophylactic measure. Delaying the onset of the at least one symptom can also be considered prevention or prophylaxis.

The term “prophylactically effective amount” refers to the amount of an agent (or combination) sufficient to prevent the occurrence or onset of one or more symptoms associated with a particular disorder. In some embodiments, the “prophylactically effective amount” is the amount of an agent (or combination) that inhibits or prevents establishment of a self-replicating infection with an infectious agent, such as an immunodeficiency virus, for example the Human Immunodeficiency Virus (HIV).

Post-exposure prophylaxis (PEP) is the prevention or inhibition of an immunodeficiency virus infection, wherein the active agent(s) are administered after a potential exposure to an immunodeficiency virus such as HIV. The exposure can be recreational (sexual, drug related, etc.) or occupational (such as from a needle stick or contaminated blood product in the hospital setting).

Pre-exposure prophylaxis (PrEP) is the prevention or inhibition of an immunodeficiency virus infection in a host, wherein the active agent(s) are administered prior to any possible infection (e.g., prior to any exposure) of the subject with the virus.

“Protection” as used in the context of a host primate response to an immunodeficiency virus challenge is defined by the host primate being serologically negative and negative in a polymerase chain reaction (PCR) testing for viral genome.

The term “substantially complete” when referring to a reaction means that the reaction contains no greater than about 50%, no greater than about 40%, no greater than about 30%, no greater than about 20%, no greater than about 10%, no greater than about 5%, no greater than about 4%, no greater than about 3%, no greater than about 2%, no greater than about 1%, no greater than about 0.5%, no greater than about 0.1%, or no greater than about 0.05% of a starting material left.

The term “substantially free” when referring to a composition that is “substantially free” of a compound means that the composition contains no greater than about 20% by weight, no greater than about 10% by weight, no greater than about 5% by weight, no greater than about 3% by weight, no greater than about 1% by weight, no greater than about 0.5% by weight, no greater than about 0.2% by weight, no greater than about 0.1% by weight, no greater than about 0.01% by weight, no greater than about 0.001% by weight, or no greater than about 0.0001% by weight of the compound.

The term “substantially pure” when referring to a compound or composition means that the compound or composition has a purity of no less than about 80% by weight, no less than about 90% by weight, no less than about 95% by weight, no less than about 96% by weight, no less than about 97% by weight, no less than about 98% by weight, no less than about 99% by weight, no less than about 99.5% by weight, no less than about 99.9% by weight, no less than about 99.95% by weight, no less than about 99.99% by weight, g no less than about 99.995% by weight, no less than about 99.999% by weight, no less than about 99.9995% by weight, or no less than about 99.9999% by weight, as determined by HPLC.

The terms “process” and “method” are used interchangeably to refer to a method disclosed herein for a compound preparation. Modifications to the processes and methods disclosed herein (e.g., starting materials, reagents, protecting groups, solvents, temperatures, reaction times, and/or purification) that are well known to those of ordinary skill in the art are also encompassed by the disclosure.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups are optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like.

The term “alkylene” or “alkanediyl” as used herein, alone or in combination, refers to a straight chain saturated or unsaturated hydrocarbon attached at two positions, such as methylene (—CH₂—), ethylene (—CH₂CH₂—), and propylene (—CH₂CH₂CH₂—). “Alkylene” thus consists of units chosen from —CH₂— and —CH═. Representative alkylenes include —CH₂—, —CH₂CH₂—, —CH═CH—, —CH₂CH₂CH₂—, —CH₂CH═CH—, and —CH═CH—CH═CH—. Alkylenes can be characterized by the count of atoms in the chain; thus, the representative alkylenes have 1, 2, 2, 3, 3, and 4 atoms, respectively.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, when alone, refer to an amino group as described below attached to the parent molecular moiety through a carbonyl group, or vice versa. The terms “amido” and “carbamoyl,” as used herein, when in combination, refer to either of —C(O)NH— and —NHC(O)—. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′ wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which is optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term “aryloxy” as used herein, alone or in combination, refers to an aryl group attached to the parent molecular moiety through an oxy.

The term “carbamate,” as used herein, alone or in combination, refers to an ester of carbamic acid (—NHCOO—) which may be attached to the parent molecular moiety from either the nitrogen or acid end, and which is optionally substituted as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. In certain embodiments, said cycloalkyl will comprise a spirocycle ring system. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1.1.1]pentane, camphor, adamantane, and bicyclo [3.2.1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and one, two, or three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroalkylene,” as used herein, alone or in combination, refers to an alkylene in which either one or both of the following hold: (a) one or more —CH₂— groups is substituted with —NH— groups, and/or (b) one or more —CH=groups is substituted with —N=groups. Representative heteroalkylenes include —CH₂NH—, —CH═NH—, —NHCH₂CH₂—, —CH₂NHCH₂—, —NHCH═CH—, —NHCH₂CH₂CH₂—, —CH═CH—N═CH, and —CH═CH—CH═N—. As with alkylenes, heteroalkylenes can be characterized by the count of atoms in the chain; thus, the representative alkylenes have 2, 2, 3, 3, 3, 4, 4, and 4 atoms, respectively.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings wherein heteroaryl rings are fused with other heteroaryl rings wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridinyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member wherein each said heteroatom may be independently chosen from nitrogen, oxygen, and sulfur. In certain embodiments, said heterocycloalkyl will comprise a spirocycle ring system. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups is optionally substituted unless specifically prohibited.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The phrase “in the main chain” refers to the longest contiguous or adjacent chain of carbon atoms starting at the point of attachment of a group to the compounds of any one of the formulas disclosed herein.

The phrase “linear chain of atoms” refers to the longest straight chain of atoms independently chosen from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which is optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′ wherein R and R′ are independently chosen from hydrogen and lower alkyl, either of which is optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “spirocycle ring system” refers to a polycyclic ring system comprising two rings such that a single atom is common to both rings.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “trihalomethoxy” refers to a X₃CO— group where X is a halogen.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}” and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. These carbon number indicators may precede or follow the chemical groups or class it modifies and it may or may not be enclosed in parenthesis, without signifying any change in meaning.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently chosen from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which is optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Provided is a method of producing phosphoramidate-containing compounds using a novel variation of the Mitsunobu coupling. Unlike state-of-the-art approaches, the reaction occurs expediently and under mild conditions. For phosphonates, the desired phosphoramidate can be generated in less than 30 minutes. For phosphates, the desired phosphoramidate can be generated in less than 1 minute. A single water extraction following reaction completion is all that is required to isolate the product in good yields (>60-70%) and with sufficient purity for additional reaction(s).

Provided is a method for preparing a compound of Formula I

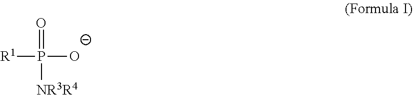

wherein

R¹ is chosen from —R² and —OR²;

R² is chosen from alkyl_{(C≤12)}, cycloalkyl_{(C≤12)}, heterocycloalkyl_{(C≤12)}, aryl_{(C≤12)}, and heteroaryl_{(C≤12)}, or a substituted version of any of these groups;

R³ is chosen from hydrogen, alkyl_{(C≤18)}, and substituted alkyl_{(C≤18)}; and

R⁴ is chosen from alkyl_{(C≤18)}, aryl_{(C≤18)}, aralkyl_{(C≤18)}, heteroaralkyl_{(C≤18)}, alkanediyl_{(C≤18)}-alkoxy_{(C≤18)}, cycloalkyl_{(C≤18)}, alkanediyl_{(C≤18)}-cycloalkyl_{(C≤18)}, or a substituted version of any of these groups; or

R³ and R⁴, together with the intervening nitrogen, combine to form a 5-7 membered heterocycloalkyl;

said method comprising:

treating a compound of Formula II

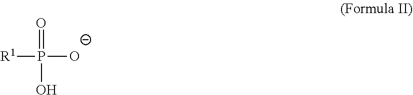

with a dialkylazodicarboxylate; a phosphine reagent; and an amine of Formula III

NHR³R⁴ (Formula III)
in an aprotic solvent to yield a compound of Formula I.

In some embodiments, R¹ is —R². In some embodiments, R¹ is —OR².

In some embodiments, R² is chosen from aryl_{(C≤12)}, aralkyl_{(C≤12)}, heteroaryl_{(C≤12)}, cycloalkyl_{(C≤12)}, and heterocycloalkyl_{(C≤12)}, or a substituted version of any of these groups.

In some embodiments, R² is aryl_{(C≤12)} or substituted aryl_{(C≤12)}. In some embodiments, R² is naphthalen-1-yl or substituted naphthalen-1-yl.

In some embodiments, R² is 1-(benzyloxy)-2-oxopiperidin-3-yl or 1-acetoxy-2-oxopiperidin-3-yl.

In some embodiments, the compound of Formula II is fludarabine monophosphate.

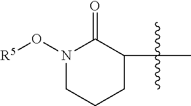

In some embodiments, R¹ is Wherein R⁵ is hydrogen, alkyl_{(C≤12)}, aryl_{(C≤12)}, heteroaryl_{(C≤12)}, aralkyl_{(C≤12)}, heteroaralkyl_{(C≤12)}, or acyl_{(C≤12)}, or a substituted version of any of these groups.

In some embodiments, R⁵ is chosen from hydrogen, acyl_{(C≤12)}, substituted acyl_{(C≤12)}, aralkyl_{(C≤12)}, and substituted aralkyl_{(C≤12)}.

In some embodiments, R⁵ is aryl_{(C≤12)} methyl.

In some embodiments, R⁵ is hydrogen.

In some embodiments, R⁵ is acyl_{(C≤12)} or substituted acyl_{(C≤12)}. In some embodiments, R⁵ is acyl_{(C≤12)}. In some embodiments, R⁵ is acetyl.

In some embodiments, R⁵ is chosen from aralkyl_{(C≤12)}, and substituted aralkyl_{(C≤12)}. In some embodiments, R⁵ is chosen from aralkyl_{(C≤12)}. In some embodiments, R⁵ is benzyl.

In some embodiments, R³ is chosen from hydrogen and methyl. In some embodiments, R³ is hydrogen. In some embodiments, R³ is methyl.

In some embodiments, R⁴ is aralkyl_{(C≤12)} or substituted aralkyl_{(C≤12)}.

In some embodiments, R⁴ is aralkyl_{(C≤12)}. In some embodiments, R⁴ is benzyl.

In some embodiments, R⁴ is substituted aralkyl_{(C≤12)}. In some embodiments, R⁴ is chosen from 3-fluorobenzyl, 4-fluorobenzyl, 2,4-difluorobenzyl, 2,6-difluorobenzyl, 3,4-difluorobenzyl, 2-aminobenzyl, and 3-hydroxybenzyl.

In some embodiments, R⁴ is heteroaralkyl_{(C≤12)} or substituted heteroaralkyl_{(C≤12)}. In some embodiments, R⁴ is heteroaralkyl_{(C≤12)}. In some embodiments, R⁴ is chosen from pyridine-2-ylmethyl and pyridine-3-ylmethyl.

In some embodiments, R⁴ is alkanediyl_{(C≤12)}-alkoxy_{(C≤18)} or alkanediyl_{(C≤12)}-alkoxy_{(C≤18)}. In some embodiments, R⁴ is alkanediyl_{(C≤12)}-alkoxy_{(C≤18)}.

In some embodiments, R⁴ is alkanediyl_{(C≤12)}-cycloalkyl_{(C≤12)} or substituted alkanediyl_{(C≤12)}-cycloalkyl_{(C≤12)}.

In some embodiments, R⁴ is alkanediyl_{(C≤12)}-cycloalkyl_{(C≤12)}. In some embodiments, R⁴ is —CH₂-cyclopropyl, —CH₂-cyclobutyl, or —CH₂-cyclohexyl.

In some embodiments, R⁴ is cycloalkyl_{(C≤12)}. In some embodiments, R⁴ is cyclopropyl or cyclobutyl.

In some embodiments, R⁴ is alkyl_{(C≤12)} or substituted alkyl_{(C≤12)}. In some embodiments, R⁴ is alkyl_{(C≤12)}.

In some embodiments, R⁴ is alkyl_{(C≤12)}. In some embodiments, R⁴ is dodecyl. In some embodiments, R⁴ is methyl.

In some embodiments, R⁴ is substituted alkyl_{(C≤12)}. In some embodiments, R⁴ is 2-(pyrrolidine-1-yl)ethyl or prop-2-yn-1-yl.

In some embodiments, R⁴ is aryl_{(C≤12)} or substituted aryl_{(C≤12)}. In some embodiments, R⁴ is substituted aryl_{(C≤12)}. In some embodiments, R⁴ is 3-fluorophenyl, 4-fluorophenyl, 2,4-difluorophenyl, 2,6-difluorophenyl, 3,4-difluorophenyl, 2-aminophenyl, and 3-hydroxyphenyl. In some embodiments, R⁴ is 4-fluorophenyl.

In some embodiments, R³ and R⁴, together with the intervening nitrogen, combine to form a 5-7 membered heterocycloalkyl. In some embodiments, R³ and R⁴, together with the intervening nitrogen, combine to form a heterocycloalkyl chosen from pyrrolidinyl and piperidinyl, or a substituted version of either of these groups.

In some embodiments, the amine of formula III is an amino acid. In some embodiments, the amine of formula III is chosen from histidine (H), arginine (R), alanine (A), isoleucine (I), cysteine (C), aspartic acid (D), leucine (L), glutamine (Q), asparagine (N), lysine (K), glycine (G), glutamic acid (E), methionine (M), proline (P), serine (S), phenylalanine (F), tyrosine (Y), selenocysteine (U), threonine (T), tryptophan (W), and valine (V). In some embodiments, the carboxyl group of the amino acid is protected as the corresponding ester. In some embodiments, the ester is prepared using a C₁₋₆ alcohol. In some embodiments, the amine of formula III is an isopropyl ester of an amino acid.

In some embodiments, the dialkylazodicarboxylate is chosen from diisopropyl azodicarboxylate (DIAD), diethyl azodicarboxylate (DEAD), and di-2-methoxyethyl azodicarboxylate (DMEAD). In some embodiments, the dialkylazodicarboxylate is DIAD.

In some embodiments, the phosphine reagent is triphenylphosphine or tributylphosphine. In some embodiments, the phosphine reagent is triphenylphosphine. In some embodiments, the reagent is triethylphosphite or triphenyl phosphite. In some embodiments, the reagent is (2-hydroxybenzyl)diphenylphosphine oxide.

In some embodiments, the ratio of dialkylazodicarboxylate to the phosphine reagent is about 1:1.

In some embodiments, the ratio of compound of Formula II and the amine of Formula III is from about 1:1 to about 1:3.

In some embodiments, the ratio of compound of Formula II and the amine of Formula III is about 1:2.

In some embodiments, the aprotic solvent is chosen from dichloromethane, chloroform, ethyl acetate, tetrahydrofuran, acetone, dimethylformamide, acetonitrile, and dimethylsulfoxide. In some embodiments, the aprotic solvent is dichloromethane. In some embodiments, the aprotic solvent is chloroform.

In some embodiments, the triphenylphosphine and the dialkylazodicarboxylate are combined to form a betaine prior to treatment with the compound of Formula II and/or the compound of Formula III.

In some embodiments, the reaction is conducted in the presence of 1,8-diazabicyclo[5.4.0]undec-7-ene (DBU). In some embodiments, from about 0.75-3 equivalents, such as about 0.75-1 equivalents of DBU is used for phosphate reactions and 2 equivalents of DBU are used for phosphonate reactions.

Also provided is a compound of Formula I prepared by any of the methods described herein.

In some embodiments, the method further comprises converting a compound of Formula I to a compound of Formula IV

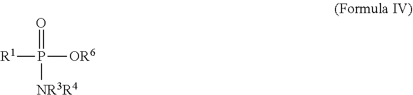

wherein

R³ is chosen from hydrogen, alkyl_{(C≤18)}, and substituted alkyl_{(C≤18)};

R⁴ is chosen from alkyl_{(C≤18)}, aryl_{(C≤18)}, aralkyl_{(C≤18)}, heteroaralkyl_{(C≤18)}, alkanediyl_{(C≤18)}-alkoxy_{(C≤18)}, cycloalkyl_{(C≤18)}, alkanediyl_{(C≤18)}-cycloalkyl_{(C≤18)}, or a substituted version of any of these groups; or

R³ and R⁴, together with the intervening nitrogen, combine to form a 5-7 membered heterocycloalkyl;

R⁶ is hydrogen, or alkyl_{(C≤12)}, acyl_{(C≤12)}, aralkyl_{(C≤12)}, heteroaralkyl_{(C≤12)}, or a substituted version of any of these groups; or -L₁-R⁷ wherein L₁ is alkanediyl_{(C≤8)} or substituted alkanediyl_{(C≤8)}; and R⁷ is acyl_{(C≤12)}, acyloxy_{(C≤12)}, acylthio_{(C≤12)}, —C(O)-alkoxy_{(C≤12)}, —OC(O)-heterocycloalkanediyl_{(C≤12)}-heterocycloalkyl_{(C≤12)}, or a substituted version of any of these groups, or R⁷ and R⁴ are taken together and are -alkanediyl_{(C≤12)}-arenediyl_{(C≤12)}- or substituted alkanediyl_{(C≤12)}-arenediyl_{(C≤12)}.

Also provided is a compound of Formula IV prepared by any of the methods described herein.

Also provided is a compound chosen from
N-benzyl-P-(1-(benzyloxy)-2-oxopiperidin-3-yl)phosphonamidic acid;
P-(1-(benzyloxy)-2-oxopiperidin-3-yl)-N-(pyridin-2-ylmethyl)phosphonamidic acid;
P-(1-(benzyloxy)-2-oxopiperidin-3-yl)-N-(pyridin-3-ylmethyl)phosphonamidic acid;
P-(1-(benzyloxy)-2-oxopiperidin-3-yl)-N-(3-fluorobenzyl)phosphonamidic acid;
P-(1-(benzyloxy)-2-oxopiperidin-3-yl)-N-(4-fluorobenzyl)phosphonamidic acid;
P-(1-(benzyloxy)-2-oxopiperidin-3-yl)-N-(2,4-difluorobenzyl)phosphonamidic acid;
P-(1-(benzyloxy)-2-oxopiperidin-3-yl)-N-(3,4-difluorobenzyl)phosphonamidic acid;
P-(1-(benzyloxy)-2-oxopiperidin-3-yl)-N-methyl-N-(2-(pyrrolidin-1-yl)ethyl phosphoramidic acid;
P-(1-(benzyloxy)-2-oxopiperidin-3-yl)-N-(prop-2-yn-1-yl)phosphonamidic acid;
P-(1-(benzyloxy)-2-oxopiperidin-3-yl)-N-(4-fluorophenyl)phosphonamidic acid;
N-(2-(aminomethyl)phenyl)-P-(1-(benzyloxy)-2-oxopiperidin-3-yl)phosphonamidic acid;
P-(1-(benzyloxy)-2-oxopiperidin-3-yl)-N-(3-hydroxybenzyl)phosphonamidic acid;
naphthalen-1-yl hydrogen benzylphosphoramidate;
naphthalen-1-yl hydrogen (pyridin-2-ylmethyl)phosphoramidate;
naphthalen-1-yl hydrogen (4-fluorobenzyl)phosphoramidate;
naphthalen-1-yl hydrogen (2,6-difluorobenzyl)phosphoramidate;
naphthalen-1-yl hydrogen (cyclohexylmethyl)phosphoramidate;
naphthalen-1-yl hydrogen cyclopropylphosphoramidate;
isopropyl (hydroxy(naphthalen-1-yloxy)phosphoryl)-L-alaninate;
((2S,3R,4R,5R)-5-(6-amino-2-fluoro-9H-purin-9-yl)-3,4-dihydroxytetrahydrofuran-2-yl)methyl hydrogen benzylphosphoramidate;
((2S,3R,4R,5R)-5-(6-amino-2-fluoro-9H-purin-9-yl)-3,4-dihydroxytetrahydrofuran-2-yl)methyl hydrogen (pyridin-2-ylmethyl)phosphoramidate;
((2S,3R,4R,5R)-5-(6-amino-2-fluoro-9H-purin-9-yl)-3,4-dihydroxytetrahydrofuran-2-yl)methyl hydrogen cyclopropylphosphoramidate;
isopropyl ((((2S,3R,4R,5R)-5-(6-amino-2-fluoro-9H-purin-9-yl)-3,4-dihydroxytetrahydrofuran-2-yl)methoxy)(hydroxy)phosphoryl)-D-alaninate;
N-benzyl-P-(1-hydroxy-2-oxopiperidin-3-yl)phosphonamidic acid;
P-(1-acetoxy-2-oxopiperidin-3-yl)-N-benzylphosphonamidic acid;
N-dodecyl-P-(1-hydroxy-2-oxopiperidin-3-yl)phosphonamidic acid;
P—((((S)-1-(6-amino-9H-purin-9-yl)propan-2-yl)oxy)methyl)-N-methylphosphonamidic acid;
1-(6-amino-9H-purin-9-yl)propan-2-yl)oxy)methyl)-N-methylphosphonamidic acid;
P-((2-(6-amino-9H-purin-9-yl)ethoxy)methyl)-N-methylphosphonamidic acid; and
P-((2-(6-amino-9H-purin-9-yl)ethoxy)methyl)-N,N-dimethylphosphonamidic acid, or

a salt thereof.

Also provided is a compound of Formula V

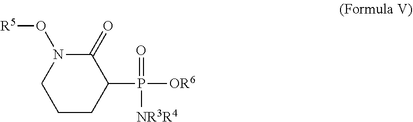

or a salt thereof, wherein

R³ is chosen from hydrogen, alkyl_{(C≤18)}, and substituted alkyl_{(C≤18)};

R⁴ is chosen from alkyl_{(C≤18)}, aryl_{(C≤18)}, aralkyl_{(C≤18)}, heteroaralkyl_{(C≤18)}, alkanediyl_{(C≤18)}-alkoxy_{(C≤18)}, cycloalkyl_{(C≤18)}, alkanediyl_{(C≤18)}-cycloalkyl_{(C≤18)}, or a substituted version of any of these groups; or

R³ and R⁴, together with the intervening nitrogen, combine to form a 5-7 membered heterocycloalkyl;

R⁵ is hydrogen, alkyl_{(C≤12)}, aryl_{(C≤12)}, heteroaryl_{(C≤12)}, aralkyl_{(C≤12)}, heteroaralkyl_{(C≤12)}, or acyl_{(C≤12)}, or a substituted version of any of these groups;

R⁶ is hydrogen, or alkyl_{(C≤12)}, acyl_{(C≤12)}, aralkyl_{(C≤12)}, heteroaralkyl_{(C≤12)}, or a substituted version of any of these groups; or -L₁-R⁷ wherein L₁ is alkanediyl_{(C≤8)} or substituted alkanediyl_{(C≤8)}; and R⁷ is acyl_{(C≤12)}, acyloxy_{(C≤12)}, acylthio_{(C≤12)}, —C(O)-alkoxy_{(C≤12)}, —OC(O)-heterocycloalkanediyl_{(C≤12)}-heterocycloalkyl_{(C≤12)}, or a substituted version of any of these groups, or R⁷ and R⁴ are taken together and are -alkanediyl_{(C≤12)}-arenediyl_{(C≤12)}- or substituted alkanediyl_{(C≤12)}-arenediyl_{(C≤12)}.

In certain embodiments, at least one of R³ and R⁴ is chosen from H and alkyl_{(C≤18)}. In certain further embodiments, at least one of R³ and R⁴ is chosen from H and CH₃. In certain further embodiments, at least one of R³ and R⁴ is H.

In certain embodiments, at least one of R³ and R⁴ is chosen from alkyl_{(8≤C≤18)} and substituted alkyl_{(8≤C≤18)}. In certain embodiments, at least one of R³ and R⁴ is chosen from alkyl_{(10≤C≤16)} and substituted alkyl_{(10≤C≤16)}. In certain embodiments, at least one of R³ and R⁴ is chosen from alkyl_{(12≤C≤16)} and substituted alkyl_{(12≤C≤16)}. In certain embodiments, at least one of R³ and R⁴ is chosen from alkyl_{(12≤C≤14)} and substituted alkyl_{(12≤C≤14)}. In certain embodiments, at least one of R³ and R⁴ is chosen from decyl, dodecyl, tetradecyl, hexadecyl, and octadecyl. In certain embodiments, at least one of R³ and R⁴ is dodecyl.

In certain embodiments, R³ is H; and R⁴ is chosen from alkyl_{(10≤C≤16)} and substituted alkyl_{( ≤C≤16)}. In certain embodiments, R³ is H; and R⁴ is chosen from alkyl_{(12≤C≤16)} and substituted alkyl_{(12≤C≤16)}. In certain embodiments, R³ is H; and R⁴ is alkyl_{(10≤C≤16)}. In certain embodiments, R³ is H; and R⁴ is chosen from decyl, dodecyl, tetradecyl, hexadecyl, and octadecyl. In certain embodiments, R³ is H; and R⁴ is dodecyl.

In certain embodiments, at least one of R³ and R⁴ is chosen from aralkyl_{(C≤18)}, heteroaralkyl_{(C≤18)}, and alkanediyl_{(C≤18)}-cycloalkyl_{(C≤18)}. In certain further embodiments, at least one of R³ and R⁴ is chosen from —CH₂-aryl_{(C≤18)}, —CH₂-heteroaryl_{(C≤18)}, and —CH₂— cycloalkyl_{(C≤18)}. In certain further embodiments, at least one of R³ and R⁴ is chosen from —CH₂-phenyl, —CH₂-pyridinyl, and —CH₂-cycloalkyl_{(C≤6)}.

In some embodiments, R⁶ is hydrogen.

In some embodiments, R⁶ is aralkyl_{(C≤12)}, heteroaralkyl_{(C≤12)}, or a substituted version of any of these groups. In some embodiments, R⁶ is heteroaralkyl_{(C≤12)} or substituted heteroaralkyl_{(C≤12)}. In some embodiments, R⁶ is (5-nitrofuran-2-yl)methyl, (1-methyl-2-nitro-1H-imidazol-5-yl)methyl, or (5-nitrothiophen-2-yl)methyl.

In some embodiments, R⁶ is substituted heteroaralkyl_{(C≤12)}.

In some embodiments, R⁶ is alkyl_{(C≤12)} or substituted alkyl_{(C≤12)}. In some embodiments, R⁶ is substituted alkyl_{(C≤12)}. In some embodiments, R⁶ is 2-cyanoethyl.

In some embodiments, R⁷ is acyloxy_{(C≤12)}, substituted acyloxy_{(C≤12)}, acylthio_{(C≤12)}, or substituted acylthio_{(C≤12)}.

In some embodiments, R⁷ is acyloxy_{(C≤12)} or substituted acyloxy_{(C≤12)}. In some embodiments, R⁷ is acyloxy_{(C≤12)}. In some embodiments, R⁷ is —OC(O)C(CH₃)₃.

In some embodiments, R⁷ is acylthio_{(C≤12)} or substituted acylthio_{(C≤12)}. In some embodiments, R⁷ is acylthio_{(C≤12)}. In some embodiments, R⁷ is —SC(O)C(CH₃)₃.

In some embodiments, R⁷ is —OC(O)-heterocycloalkanediyl_{(C≤12)}-heterocycloalkyl_{(C≤12)} or substituted —OC(O)-heterocycloalkanediyl_{(C≤12)}-heterocycloalkyl_{(C≤12)}. In some embodiments, R⁷ is —OC(O)-heterocycloalkanediyl_{(C≤12)}-heterocycloalkyl_{(C≤12)}.

In some embodiments, L₁ is alkanediyl_{(C≤8)}. In some embodiments, L₁ is methanediyl or ethanediyl.

Also provided is a compound chosen from
N-benzyl-P-(1-hydroxy-2-oxopiperidin-3-yl)phosphonamidic acid;
3-((benzylamino)(2-(pivaloylthio)ethoxy)phosphoryl)-2-oxopiperidin-1-yl acetate;
3-((benzylamino)((S-nitrofuran-2-yl)methoxy)phosphoryl)-2-oxopiperidin-1-yl acetate;
3-((benzylamino)((5-nitrothiophen-2-yl)methoxy)phosphoryl)-2-oxopiperidin-1-yi acetate;
3-((benzylamino)((1-methyl-2-nitro-1H-imidazol-5-yl)methoxy)phosphoryl)-2-oxopiperidin-1-yl acetate;
(((1-hydroxy-2-oxopiperidin-3-yl){(pyridin-2-ylmethyl)amino)phosphoryl)oxy)methyl pivalate;
((((3-fluorobenzyl)amino)(1-hydroxy-2-oxopiperidin-3-yl)phosphoryl)oxy)methyl pivalate;
((((3,4-difluornhenzyl)amino)(1-hydro:xy-2-oxopiperidin-3-yl)phosphoryl)oxy)methyl pivalate;
((((2,4-difluorobenzyl)amino)(1-hydroxy-2-oxopiperidin-3-yl)phosphoryl)oxy)methyl pivalate;
((((Cyclohexylmethyl)amino)(1-hydroxy-2-oxopiperidin-3-yl)phosphoryl)oxy)methyl pivalate;
((((2,6-difluorobenzyl)amino)(1-hydroxy-2-oxopiperidin-3-yl)phosphoryl)oxy)methyl pivalate;
((((4-fluorobenzyl)amino)(1-hydroxy-2-oxopiperidin-3-yl)phosphoryl)oxy)methyl pivalate;
((((Cyclopropylmethyl)amino)(1-hydroxy-2-oxopiperidin-3-yl)phosphoryl)oxy)methyl pivalate;
(((Cyclobutylamino)(1-hydroxy-2-oxopiperidin-3-yl)phosphoryl)oxy)methyl pivalate;
2-oxo-3-((2-(pivaloylthio)ethoxy)((pyridin-2-ylmethyl)amino)phosphoryl)piperidin-1-yl acetate;
3-(((4-fluorobenzyl)amino)((5-nitrofuran-2-yl)methoxy)phosphoryl)-2-oxopiperidin-1-yl acetate;
2-cyanoethyl N-benzyl-P-(1-hydroxy-2-oxopiperidin-3-yl)phosphonamidate; and
(((benzylamino)(1-hydroxy-2-oxopiperidin-3-yl)phosphoryl)oxy)methyl [1,4′-bipiperidine]-1′-carboxylate,

or a salt thereof.

In certain embodiments, the compound of Formula I, Formula IV, or Formula V is substantially pure.

In certain embodiments, the compound of Formula I, Formula IV, or Formula V is substantially free of triphenylphosphine oxide. In certain embodiments, the compound of Formula I, Formula IV, or Formula V is substantially free of dimer.

In certain embodiments, the compound of Formula I, Formula IV, or Formula V is prepared in a yield that is no less than about 50%, no less than about 55%, no less than about 60%, no less than about 65%, no less than about 70%, no less than about 75%, no less than about 80%, no less than about 85%, no less than about 90%, or no less than about 95%, wherein the yield is calculated based on starting material.

In certain embodiments, the total impurities in the compound of Formula I, Formula IV, or Formula V prepared by the methods provided herein are no greater than about 5% by weight, no greater than about 4% by weight, no greater than about 3% by weight, no greater than about 2.5% by weight, no greater than about 2% by weight, no greater than about 1.5% by weight, no greater than about 1% by weight, no greater than about 0.5% by weight, or no greater than about 0.1% by weight. In certain embodiments, the impurity is detectable by HPLC (high performance liquid chromatography).

In some embodiments, the method for preparing a compound of Formula I, further comprises combining the compound of Formula I with a pharmaceutically acceptable carrier to produce a pharmaceutical composition.

In some embodiments, the method for preparing a compound of Formula IV, further comprises combining the compound of Formula IV with a pharmaceutically acceptable carrier to produce a pharmaceutical composition.

Also provided is a method of inhibition of enolase, the method comprising the step of contacting enolase (ENO) with a compound of Formula I. Also provided is a method of inhibition of enolase, the method comprising the step of contacting enolase with a compound of Formula IV. Also provided is a method of inhibition of enolase, the method comprising the step of contacting enolase with a compound of Formula V, or a salt thereof. Also provided is a method of inhibition of enolase, the method comprising the step of contacting enolase with a compound, or a salt thereof, described herein. In certain embodiments, the enolase is selective for enolase 1 (ENO1). In certain embodiments, the enolase is selective for enolase 1 (ENO2).

Also provided is a method of inhibition of an enolase-mediated disease, the method comprising the administration of a therapeutically effective amount of a compound of Formula I. Also provided is a method of inhibition of an enolase-mediated disease, the method comprising the administration of a therapeutically effective amount of a compound of Formula IV. Also provided is a method of inhibition of an enolase-mediated disease, the method comprising the administration of a therapeutically effective amount of a compound of Formula V, or a salt thereof. Also provided is a method of inhibition of an enolase-mediated disease, the method comprising the administration of a therapeutically effective amount of a compound, or a salt thereof, described herein.

In certain embodiments, the enolase-mediated disease is cancer. In certain embodiments, the cancer is an ENO1-deleted cancer. In certain embodiments, the cancer is glioma. In certain embodiments, the glioma is glioblastoma.

Also provided is a compound chosen from

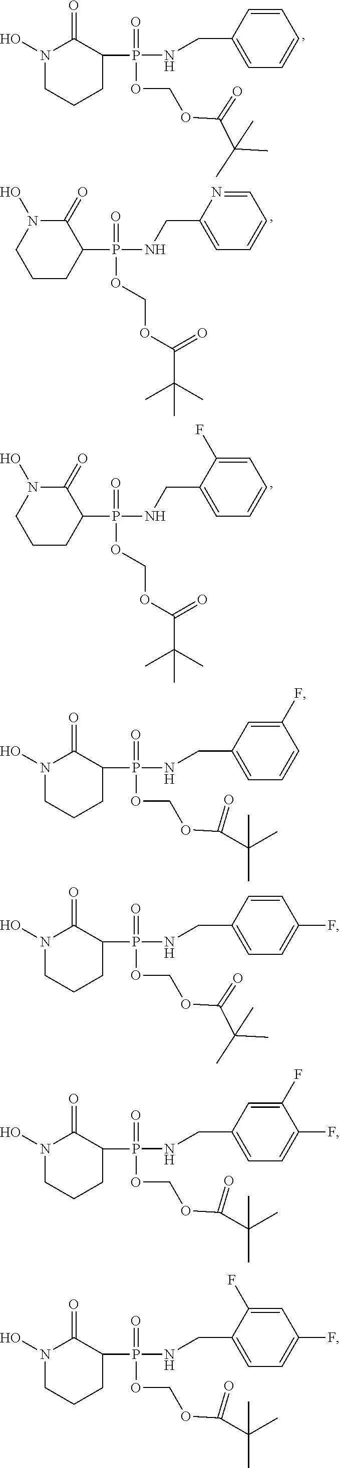

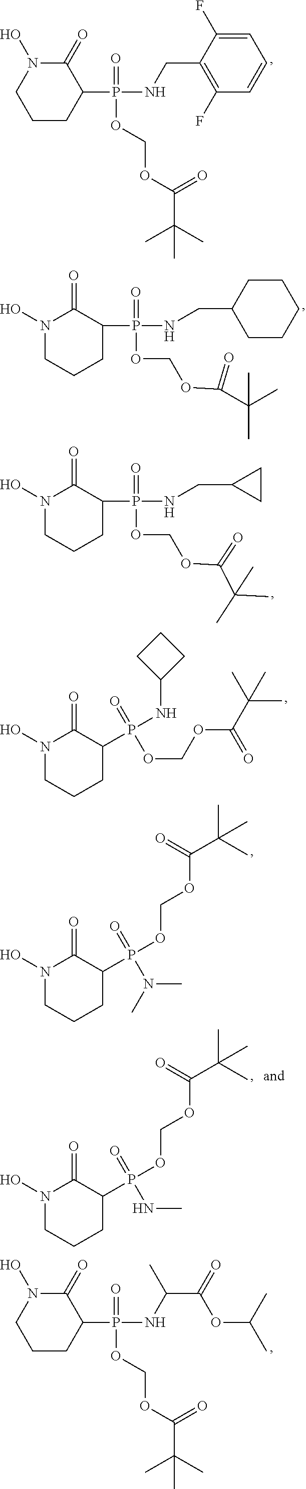

or a salt thereof.

Also provided is a method of inhibition of RNA polymerase, the method comprising the step of contacting RNA polymerase with a compound of Formula I. Also provided is a method of inhibition of RNA polymerase, the method comprising the step of contacting RNA polymerase with a compound of Formula IV. Also provided is a method of inhibition of RNA polymerase, the method comprising the step of contacting RNA polymerase with a compound of Formula V, or a salt thereof. Also provided is a method of inhibition of RNA polymerase, the method comprising the step of contacting RNA polymerase with a compound described herein, or a salt thereof. In certain embodiments, the RNA polymerase is an RNA-dependent polymerase. In certain embodiments, the RNA polymerase is nonstructural protein 5B (NS5B).

Also provided is a method of inhibition of an RNA polymerase-mediated disease, the method comprising the administration of a therapeutically effective amount of a compound of Formula I. Also provided is a method of inhibition of an RNA polymerase-mediated disease, the method comprising the administration of a therapeutically effective amount of a compound of Formula IV. Also provided is a method of inhibition of an RNA polymerase-mediated disease, the method comprising the administration of a therapeutically effective amount of a compound of Formula V, or a salt thereof. Also provided is a method of inhibition of an RNA polymerase-mediated disease, the method comprising the administration of a therapeutically effective amount of a compound described herein, or a salt thereof.

In certain embodiments, the RNA polymerase-mediated disease is hepatitis. In certain embodiments, the hepatitis is hepatitis B. In certain embodiments, the RNA polymerase-mediated disease is associated with human immunodeficiency virus (HIV). In certain embodiments, the RNA polymerase-mediated disease is chosen from acquired immunodeficiency syndrome (AIDS) and an AIDS-related disease.

Also provided is a method of treating HIV (e.g., HIV-1 and/or HIV-2) infections in a patient in need thereof, comprising administering a compound of Formula I, optionally in combination with one or more other antiretroviral agents, to the patient. Also provided is a method of treating HIV (e.g., HIV-1 and/or HIV-2) infections in a patient in need thereof, comprising administering a compound of Formula IV, optionally in combination with one or more other antiretroviral agents, to the patient. Also provided is a method of treating HIV (e.g., HIV-1 and/or HIV-2) infections in a patient in need thereof, comprising administering a compound of Formula V, or a salt thereof, optionally in combination with one or more other antiretroviral agents, to the patient. Also provided is a method of treating HIV (e.g., HIV-1 and/or HIV-2) infections in a patient in need thereof, comprising administering a compound described herein, or a salt thereof, optionally in combination with one or more other antiretroviral agents, to the patient.

Also provided is a method of preventing HIV (e.g., HIV-1 and/or HIV-2) infections in a patient in need thereof, comprising administering a compound of Formula I, optionally in combination with one or more other antiretroviral agents, to the patient. Also provided is a method of preventing HIV (e.g., HIV-1 and/or HIV-2) infections in a patient in need thereof, comprising administering a compound of Formula IV, optionally in combination with one or more other antiretroviral agents, to the patient. Also provided is a method of preventing HIV (e.g., HIV-1 and/or HIV-2) infections in a patient in need thereof, comprising administering a compound of Formula V, or a salt thereof, optionally in combination with one or more other antiretroviral agents, to the patient. Also provided is a method of preventing HIV (e.g., HIV-1 and/or HIV-2) infections in a patient in need thereof, comprising administering a compound described herein, or a salt thereof, optionally in combination with one or more other antiretroviral agents, to the patient. In some embodiments, the methods disclosed herein comprise pre-exposure prophylaxis (PrEP). In some embodiments, methods disclosed herein comprise post-exposure prophylaxis (PEP).

Also provided is a method to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP) comprising administering a compound of Formula I, optionally in combination with one or more other antiretroviral agents, to the patient. Also provided is a method to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP) comprising administering a compound of Formula IV, optionally in combination with one or more other antiretroviral agents, to the patient. Also provided is a method to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP) comprising administering a compound of Formula V, or a salt thereof, optionally in combination with one or more other antiretroviral agents, to the patient. Also provided is a method to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP) comprising administering a compound described herein, or a salt thereof, optionally in combination with one or more other antiretroviral agents, to the patient.

Also provided is a method for reducing the risk of acquiring HIV (e.g., HIV-1 and/or HIV-2) comprising administering a compound of Formula I, optionally in combination with one or more other antiretroviral agents, to the patient. In certain specific embodiments, the method is used in combination with safer sex practices. Also provided is a method for reducing the risk of acquiring HIV (e.g., HIV-1 and/or HIV-2) comprising administering a compound of Formula IV, optionally in combination with one or more other antiretroviral agents, to the patient. Also provided is a method for reducing the risk of acquiring HIV (e.g., HIV-1 and/or HIV-2) comprising administering a compound of Formula V, or a salt thereof, optionally in combination with one or more other antiretroviral agents, to the patient. In certain specific embodiments, the method is used in combination with safer sex practices. Also provided is a method for reducing the risk of acquiring HIV (e.g., HIV-1 and/or HIV-2) comprising administering a compound described herein, or a salt thereof, optionally in combination with one or more other antiretroviral agents, to the patient. In certain specific embodiments, the method is used in combination with safer sex practices.

Also provided is a method for reducing the risk of acquiring HIV (e.g., HIV-1 and/or HIV-2) comprising administering to an individual at risk of acquiring HIV a compound of Formula I, optionally in combination with one or more other antiretroviral agents. Examples of individuals at high risk for acquiring HIV include, without limitation, an individual who is at risk of sexual transmission of HIV. Also provided is a method for reducing the risk of acquiring HIV (e.g., HIV-1 and/or HIV-2) comprising administering to an individual at risk of acquiring HIV a compound of Formula IV, optionally in combination with one or more other antiretroviral agents. Examples of individuals at high risk for acquiring HIV include, without limitation, an individual who is at risk of sexual transmission of HIV. Also provided is a method for reducing the risk of acquiring HIV (e.g., HIV-1 and/or HIV-2) comprising administering to an individual at risk of acquiring HIV a compound of Formula V, or a salt thereof, optionally in combination with one or more other antiretroviral agents. Examples of individuals at high risk for acquiring HIV include, without limitation, an individual who is at risk of sexual transmission of HIV. Also provided is a method for reducing the risk of acquiring HIV (e.g., HIV-1 and/or HIV-2) comprising administering to an individual at risk of acquiring HIV a compound described herein, or a salt thereof, optionally in combination with one or more other antiretroviral agents. Examples of individuals at high risk for acquiring HIV include, without limitation, an individual who is at risk of sexual transmission of HIV.

In certain embodiments, the reduction in risk of acquiring HIV is at least about 40%, 50%, 60%, 70%, 80%, 90%, or 95%. In certain embodiments, the reduction in risk of acquiring HIV is at least about 75%. In certain embodiments, the reduction in risk of acquiring HIV is about 80%, 85%, or 90%.

Also provided is a method of treating chronic hepatitis B in a patient in need thereof, comprising administering a compound of Formula I to the patient. Also provided is a method of treating chronic hepatitis B in a patient in need thereof, comprising administering a compound of Formula IV to the patient. Also provided is a method of treating chronic hepatitis B in a patient in need thereof, comprising administering a compound of Formula V, or a salt thereof, to the patient. Also provided is a method of treating chronic hepatitis B in a patient in need thereof, comprising administering a compound described herein, or a salt thereof, to the patient.

In some embodiments, the compound is

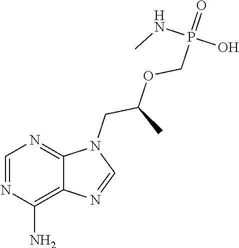 [SMILES: CNP(=O)(O)CO[C@@H](C)Cn1cnc2c(N)ncnc21]

or a salt thereof.

In some embodiments, the compound is

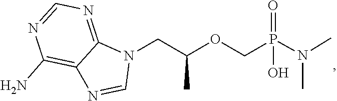 [SMILES: C[C@@H](Cn1cnc2c(N)ncnc21)OCP(=O)(O)N(C)C]

or a salt thereof.

In some embodiments, the compound is

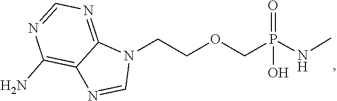 [SMILES: CNP(=O)(O)COCCn1cnc2c(N)ncnc21]

or a salt thereof.

In some embodiments, the compound is

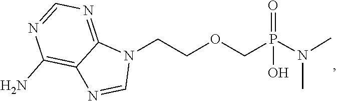 [SMILES: CN(C)P(=O)(O)COCCn1cnc2c(N)ncnc21]

or a salt thereof.

Also provided is a method of inhibition of an enzyme chosen from RNA reductase and DNA polymerase, the method comprising the step of contacting the enzyme with a compound of Formula I. Also provided is a method of inhibition of an enzyme chosen from RNA reductase and DNA polymerase, the method comprising the step of contacting the enzyme with a compound of Formula IV. Also provided is a method of inhibition of an enzyme chosen from RNA reductase and DNA polymerase, the method comprising the step of contacting the enzyme with a compound of Formula V, or a salt thereof. Also provided is a method of inhibition of an enzyme chosen from RNA reductase and DNA polymerase, the method comprising the step of contacting the enzyme with a compound described herein, or a salt thereof.

Also provided is a method of inhibition of a disease mediated by an enzyme chosen from RNA reductase and DNA polymerase, the method comprising the administration of a therapeutically effective amount of a compound of Formula I. Also provided is a method of inhibition of a disease mediated by an enzyme chosen from RNA reductase and DNA polymerase, the method comprising the administration of a therapeutically effective amount of a compound of Formula IV. Also provided is a method of inhibition of a disease mediated by an enzyme chosen from RNA reductase and DNA polymerase, the method comprising the administration of a therapeutically effective amount of a compound of Formula V, or a salt thereof. Also provided is a method of inhibition of a disease mediated by an enzyme chosen from RNA reductase and DNA polymerase, the method comprising the administration of a therapeutically effective amount of a compound described herein, or a salt thereof.

In certain embodiments, the disease mediated by an enzyme chosen from RNA reductase and DNA polymerase is a lymphoproliferative malignancy. In certain embodiments, the disease mediated by an enzyme chosen from RNA reductase and DNA polymerase is leukemia. In certain embodiments, the disease mediated by an enzyme chosen from RNA reductase and DNA polymerase is leukemia. In certain further embodiments, the leukemia is chosen from hairy cell leukemia, acute myeloid leukemia, chronic lymphocytic leukemia, and acute lymphocytic leukemia. In certain embodiments, the disease mediated by an enzyme chosen from RNA reductase and DNA polymerase is lymphoma. In certain further embodiments, the lymphoma is non-Hodgkin's lymphoma.

Also provided is a method of treating B-cell chronic lymphocytic leukemia in a patient in need thereof, comprising administering a compound of Formula I to the patient. Also provided is a method of treating B-cell chronic lymphocytic leukemia in a patient in need thereof, comprising administering a compound of Formula IV to the patient. Also provided is a method of treating B-cell chronic lymphocytic leukemia in a patient in need thereof, comprising administering a compound of Formula V, or a salt thereof, to the patient. Also provided is a method of treating B-cell chronic lymphocytic leukemia in a patient in need thereof, comprising administering a compound described herein, or a salt thereof, to the patient. In some embodiments, the patient has not responded to or whose disease has progressed during treatment with at least one standard alkylating-agent containing regimen.

In some embodiments, the compound is

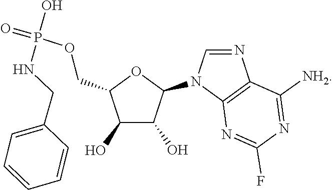 [SMILES: Nc1nc(F)nc2c1ncn2[C@@H]1O[C@@H](COP(=O)(O)NCc2ccccc2)[C@H](O)[C@H]1O]

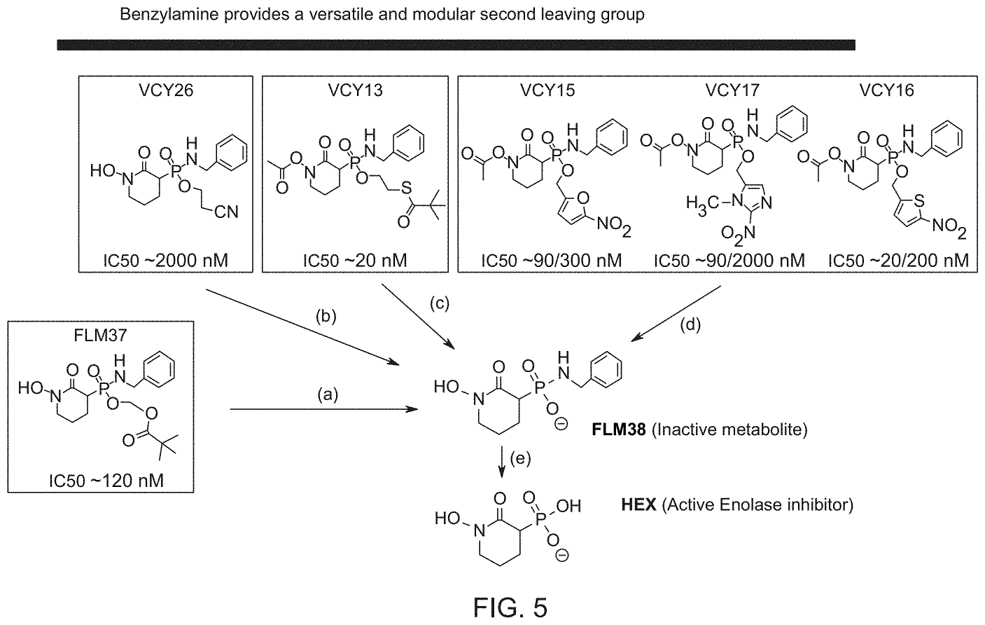

Without wishing to be bound by theory, a concept for delivery of an enolase inhibitor from a benzylamine-derived compound is presented in FIG. 5. The ester and thiolester functionalities of FLM37 and VCY13, respectively, can be cleaved by carboxyesterase and thioesterase, respectively. The cyanoethyl moeity of VCY26 can be cleaved via E2 elimination. The ((nitro)heteroaryl)methyl groups of VCY15, VCY17, and VC16 are susceptible to removal, due to formation of the corresponding hydroxylamine by the action of nitroreductase. The product that is formed from all pathways, FLM38, can be converted to the active HEX via phosphoramidase-promoted removal of the benzyl group.

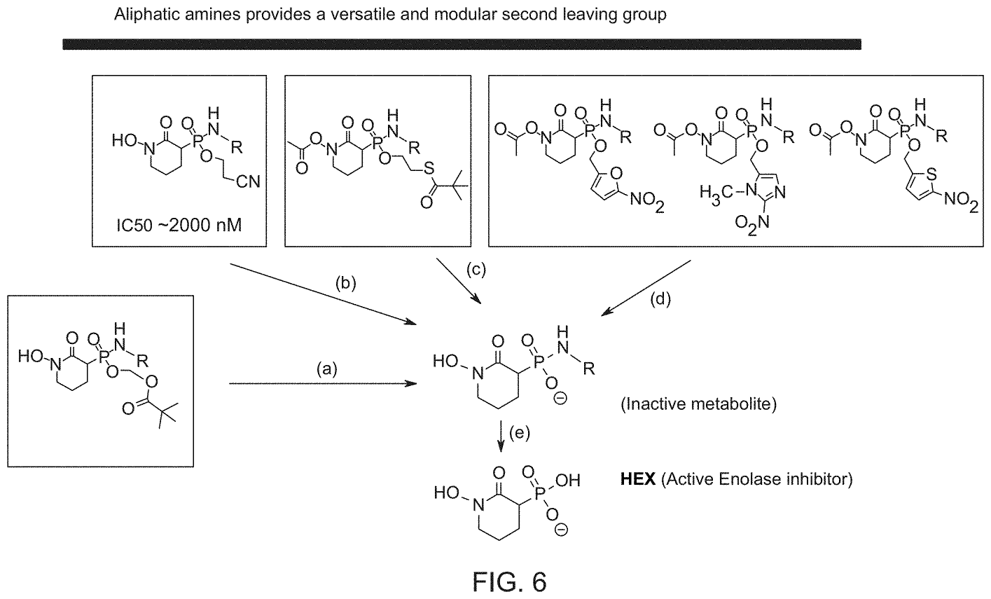

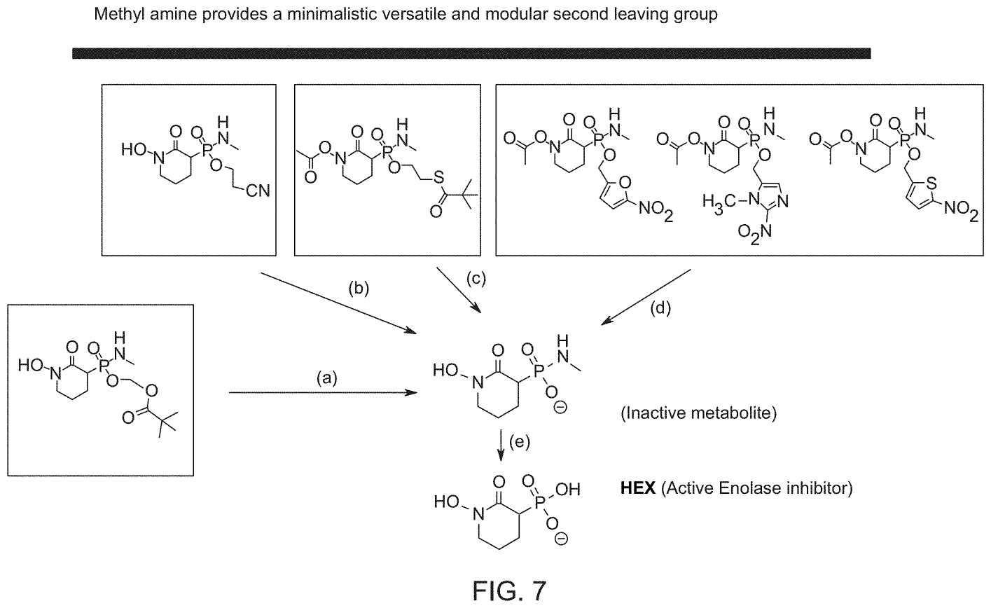

Similar behavior is depicted for aliphatic amine- and methylamine-based compounds in FIGS. 6 and 7, respectively.

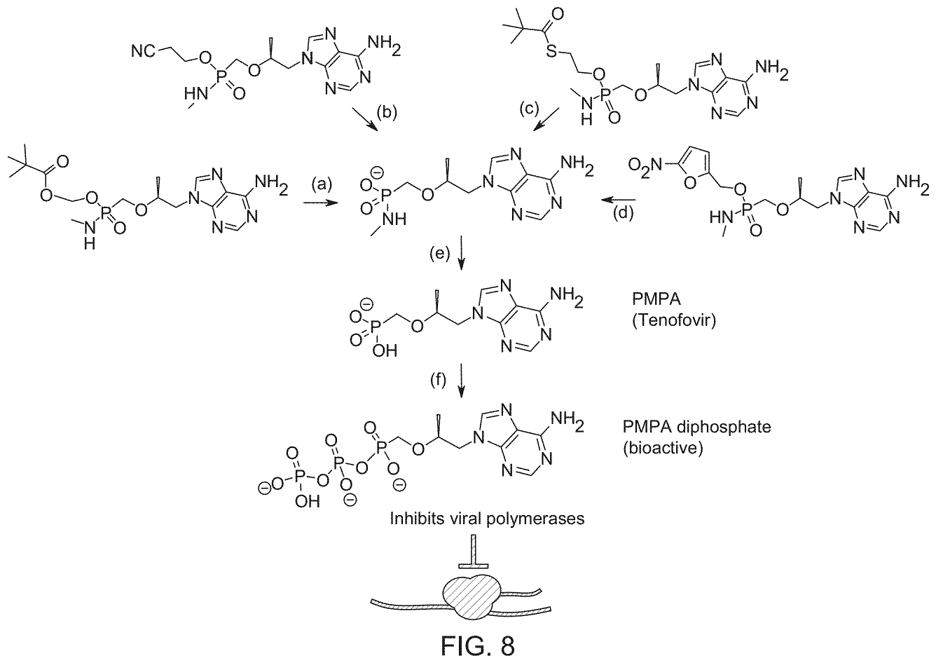

Potential behavior of purine-based compounds is depicted in FIG. 8. Removal of groups proceeds as described above to afford the monophosphate PMPA (Tenofovir). The action of nucleotide kinase provides the triphosphate PMPA diphosphate, which is the putative active species for inhibition of viral polymerases.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### General Information

¹H, ¹³C, and ³¹P, and ¹⁹F NMR were recorded on a Bruker Avance 300 MHz spectrometer in either CDCl₃ or D₂O, unless otherwise indicated. Chemical shifts were measured in ppm relative to CDCl₃ (δ=7.24 for ¹H and δ=77.0 for ¹³C) or D₂O (δ=4.80 for ¹H). NMR characterizations are reported in the following order: chemical shift, multiplicity (s=singlet, d=doublet, t=triplet, q=quartet, m=multiplet), and coupling constant (J, reported in Hertz). Where appropriate, 2-dimensional experiments (HSQC) and decoupled NMR experiments (¹H with ³¹P decoupling) were used to support assignments. BnHEX was initially synthesized according to previously published procedures. Fludarabine Monophosphate was purchased from Selleckchem. Other starting materials were purchased at the highest commercial quality from Sigma Aldrich and were used without additional purification. Centrifugations were performed using an Eppendorf Centrifuge 5810R. Mass spectra were obtained on a Waters Acquity UPLC H-Class PLUS System (A/B: A=30% MeCN in water, B=0.1% formic acid in water) using an electrospray ion source.

Abbreviations used: DCM-dichloromethane; NMP-N-methyl-2-pyrrolidone; BnHEX-(1-(benzyloxy)-2-oxopiperidin-3-yl)phosphonic acid; Fludarabine monophosphate-((2S,3R,4R,5R)-5-(6-amino-2-fluoro-9H-purin-9-yl)-3,4-dihydroxytetrahydrofuran-2-yl)methyl dihydrogen phosphate; DIAD-diisopropyl (E)-diazene-1,2-dicarboxylate=diisopropyl azodicarboxylate; DIPEA-diisopropylethylamine; HEX-(1-hydroxy-2-oxopiperidin-3-yl)phosphonic acid; MeCN-acetonitrile; MeOH-methanol; POM-Pivaloyloxymethyl; POM-Cl-pivaloyloxymethyl chloride; POMHEX-(((1-hydroxy-2-oxopiperidin-3-yl)phosphoryl)bis(oxy))bis(methylene) bis(2,2-dimethylpropanoate); TFA-trifluoroacetic acid; THF-tetrahydrofuran.

### Synthetic Procedures

Synthesis of phosphoramidates from BnHEX. All coupling reactions described for BnHEX (1) follow the same general procedure. Betaine formation: DIAD (2 equiv.) and triphenyl phosphine (2 equiv.) were combined in anhydrous DCM at 0° C. and allowed to stir to room temperature for 30 minutes. Separately, BnHEX (1 equiv.) and the indicated amine (2 equiv.) were dissolved in anhydrous DCM and then added dropwise to the betaine solution. The reaction was allowed to stir for 30 minutes. The crude reaction mixture was then transferred to a 50 mL Falcon tube, where 1 volume of water was added. The reaction was vortexed and centrifuged (4000 rpm, 4° C.) for 2 minutes. The aqueous layer was then isolated and lyophilized to a white powder, unless otherwise specified.

### Synthesis of N-benzyl-P-(1-(benzyloxy)-2-oxopiperidin-3-yl)phosphonamidic acid (2, BnFLM38)

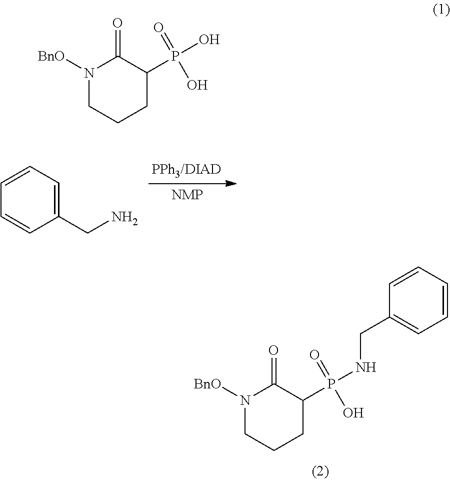 [SMILES: O=C1C(P(=O)(O)O)CCCN1OCc1ccccc1 ; NCc1ccccc1 ; O=C1C(P(=O)(O)NCc2ccccc2)CCCN1OCc1ccccc1]

Following the general procedure above, the following quantities were used: DIAD (68.17 μL, 350.59 μmol) and triphenylphosphine (43.87 mg, 350.59 μmol) in anhydrous DCM (3 mL). BnHEX (1, 50 mg, 175.29 μmol) and benzylamine (38.30 μL, 350.59 μmol) in anhydrous DCM (1 mL). Yield: 40%. Analysis by ESI+(Expected [M+H]+=375.38. Observed [M+H]+=375.35). ¹H NMR (500 MHz, D₂O) δ 7.32-7.53 (m, 10H), 4.91-4.96 (m, 2H), 4.02-4.11 (m, 2H)), 3.46-3.57 (m, 2H), 2.80-2.87 (dt, J=21.52 Hz, J=21.70 Hz, 1H), 2.07-2.13 (m, 1H), 1.92-1.98 (m, 2H), 1.72-1.79 (m, 1H). ¹³C NMR (125.7 MHz, D₂O) δ 167.97 (d, J=4.72 Hz, 1C), 141.25 (d, J=7.36 Hz, 1C), 134.52 (s, 1C), 129.92 (s, 2C), 129.15 (s, 1C), 128.73 (s, 2C), 128.60 (s, 2C), 127.59 (s, 1C), 127.00 (s, 1C), 75.54 (s, 1C), 50.11 (s, 1C), 45.28 (s, 1C), 43.64 (d, J=111.12 Hz, 1C), 22.33 (d, J=3.65 Hz, 1C), 21.65 (d, J=7.23 Hz, 1C). ³¹P NMR (202 MHz, D₂O) δ 20.91.

### Synthesis of P-(1-(benzyloxy)-2-oxopiperidin-3-yl)-N-(pyridin-2-ylmethyl)phosphonamidic acid (3)

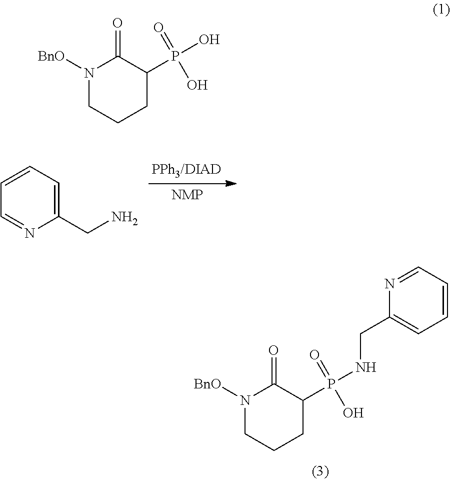 [SMILES: O=C1C(P(=O)(O)O)CCCN1OCc1ccccc1 ; NCc1ccccn1 ; O=C1C(P(=O)(O)NCc2ccccn2)CCCN1OCc1ccccc1]

Following the general procedure above, the following quantities were used: DIAD (68.17 μL, 350.59 μmol) and triphenylphosphine (43.87 mg, 350.59 μmol) in anhydrous DCM (3 mL). BnHEX (1, 50 mg, 175.29 μmol) and pyridin-2-ylmethanamine (37.83 μL, 350.59 μmol) in anhydrous DCM (1 mL). The product was lyophilized to an orange oil. Yield: 74%. Analysis by ESI+(Expected [M+H]+=376.36. Observed [M+H]+=376.35). ¹H NMR (300 MHz, D₂O) δ 8.38 (d, J=4.73 Hz, 1H), 7.83 (t, J=1.77, 1.92 Hz, 1H), 7.37-7.49 (m, 7H), 4.90 (d, J=2.02 Hz, 2H), 3.43-3.58 (m, 2H), 2.79-2.91 (dt, J=21.84 Hz, 1H), 2.06-2.14 (m, 1H), 1.91-1.99 (m, 2H), 1.70-1.81 (m, 1H). ¹³C NMR (75 MHz, D₂O) δ 167.86 (s, 1C), 159.86 (d, J=7.28 Hz, 1C), 147.91 (s, 1C), 138.34 (s, 1C), 129.84 (s, 2C), 129.11 (s, 1C), 128.70 (s, 2C), 123.71 (s, 1C), 122.76 (s, 1C), 122.07, (s, 1C), 71.16 (s, 1C), 50.04 (s, 1C), 46.42 (s, 1C), 43.18-44.65 (d, J=111.72 Hz, 1C), 22.38 (d, J=3.65 Hz, 1C), 21.54 (d, J=7.01 Hz, 1C). ³¹P NMR (121 MHz, D₂O) δ 20.60.

### Synthesis of P-(1-(benzyloxy)-2-oxopiperidin-3-yl)-N-(pyridin-3-ylmethyl)phosphonamidic acid (4)

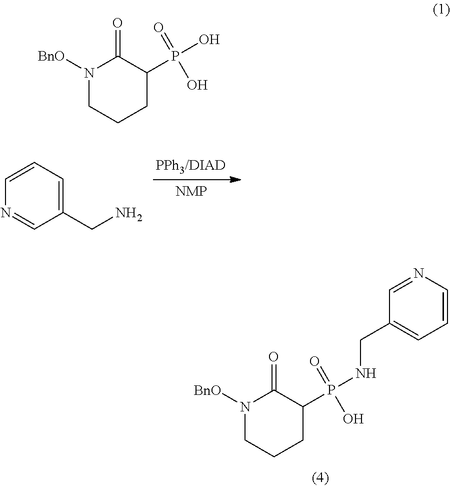 [SMILES: O=C1C(P(=O)(O)O)CCCN1OCc1ccccc1 ; NCc1cccnc1 ; O=C1C(P(=O)(O)NCc2cccnc2)CCCN1OCc1ccccc1]

Following the general procedure above, the following quantities were used: DIAD (68.17 μL, 350.59 μmol) and triphenylphosphine (43.87 mg, 350.59 μmol) in anhydrous DCM (3 mL). BnHEX (1, 50 mg, 175.29 μmol) and pyridin-3-ylmethanamine (37.83 μL, 350.59 μmol) in anhydrous DCM (1 mL). Yield: 76% Analysis by ESI+(Expected [M+H]+=376.36. Observed [M+H]+=376.35). ¹H NMR (300 MHz, D₂O) δ 8.58 (d, J=2.14 Hz, 1H), 8.38-8.40 (dd, J=5.02 Hz, 1H), 7.92-7.95 (dt, J=7.90 Hz, 1H), 7.53-7.48 (m, 5H), 4.92 (s, 2H), 4.07-4.10 (d, J=9.13 Hz, 2H), 3.47-3.55 (m, 2H), 2.76-2.88 (dt, J=21.88 Hz, 1H), 2.05-2.16 (m, 1H), 1.90-2.00 (m, 2H), 1.70-1.82 (m, 1H). ¹³C NMR (75 MHz, D₂O) 167.84 (d, J=4.44 Hz, 1C), 149.20 (s, 1C), 148.82 (s, 1C), 147.65 (s, 1C), 146.93 (s, 1C), 129.88 (s, 2C), 129.62 (s, 1C), 129.13 (s, 1C), 128.71 (s, 2C), 124.09 (s, 1C), 75.47 (s, 1C), 43.15-44.63 (d, J=110.71 Hz, 1C), 42.67 (s, 1C), 22.41 (d, J=4.10 Hz, 1C), 2.53-2.63 (d, J=7.02 Hz, 1C), 21.14-21.17 (d, J=2.48 Hz, 1C). ³¹P NMR (121 MHz, D₂O) δ 20.52.

### Synthesis of P-(1-(benzyloxy)-2-oxopiperidin-3-yl)-N-(3-fluorobenzyl)phosphonamidic acid (5)

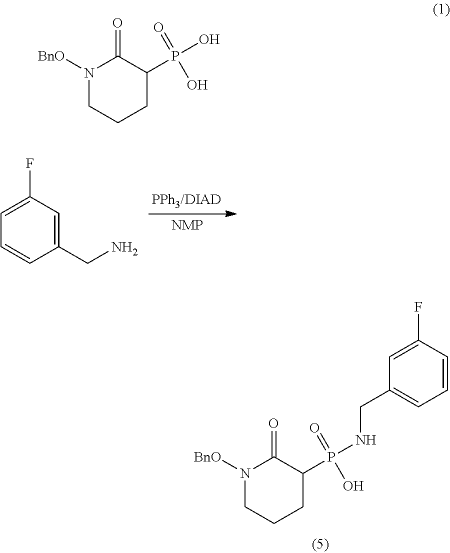 [SMILES: O=C1C(P(=O)(O)O)CCCN1OCc1ccccc1 ; NCc1cccc(F)c1 ; O=C1C(P(=O)(O)NCc2cccc(F)c2)CCCN1OCc1ccccc1]

Following the general procedure above, the following quantities were used: DIAD (86.98 μL, 441.74 μmol) and triphenylphosphine (115.86 mg, 441.74 μmol) in anhydrous DCM (3 mL). BnHEX (1, 63 mg, 220.87 μmol) and 3-fluorophenyl methanamine (50.39 μL, 441.74 μmol) in anhydrous DCM (1 mL). Yield: 61%. Analysis by ESI+(Expected [M+H]+=393.37. Observed [M+H]+=393.42). ¹H NMR (300 MHz, CDCl₃) δ 7.18-7.38 (m, 9H), 4.87 (s, 2H), 4.03 (s, 2H), 3.27-3.38 (m, 2H), 2.62-2.74 (dt, J=22.20 Hz, J=6.9 Hz, 1H), 2.07-2.13 (m, 1H), 1.79-1.88 (m, 2H), 1.55-1.59 (m, 1H). ¹³C NMR (75 MHz, CDCl₃) δ 166.80 (d, J=4.50 Hz, 1C), 161.13 (d, J=2.32 Hz 1C), 144.65 (d, J=7.36 Hz, 1C), 135.40 (s, 1C), 129.39 (s, 2C), 129.58 (s, 1C), 128.42 (s, 2C), 124.60 (d, J=2.89 Hz, 1C), 122.83 (s, 1C), 115.95 (d, J=21.89 Hz, 1C), 114.24 (d, J=21.34 Hz, 1C), 75.48 (s, 1C), 50.52 (s, 1C), 45.47 (s, 1C), 42.70-43.64 (d, J=96 Hz, 1C), 23.12 (d, J=2.84 Hz, 1C), 22.35 (d, J=8.16 Hz, 1C). ³¹P NMR (121 MHz, CDCl₃) δ 19.30. ¹⁹F NMR (282 MHz, CDCl₃) δ−112.30.

### Synthesis of P-(1-(benzyloxy)-2-oxopiperidin-3-yl)-N-(4-fluorobenzyl)phosphonamidic acid (6)

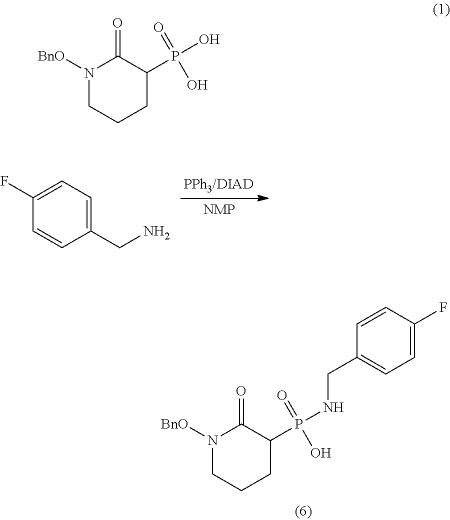 [SMILES: O=C1C(P(=O)(O)O)CCCN1OCc1ccccc1 ; NCc1ccc(F)cc1 ; O=C1C(P(=O)(O)NCc2ccc(F)cc2)CCCN1OCc1ccccc1]

Following the general procedure above, the following quantities were used: DIAD (68.17 μL, 350.59 μmol) and triphenylphosphine (43.87 mg, 350.59 μmol) in anhydrous DCM (3 mL). BnHEX (1, 50 mg, 175.29 μmol) 4-fluorophenyl methanamine (40.07 μL, 350.59 μmol) in anhydrous DCM (1 mL). Yield: 64%. Analysis by ESI+(Expected [M+H]+=393.37. Observed [M+H]+=393.38). ¹H NMR (300 MHz, D₂O) δ 7.41-7.54 (m, 5H), 7.10-7.24 (m, 4H), 4.94 (s, 2H), 4.02 (d, J=8.76 Hz, 2H), 3.46-3.60 (m, 2H), 2.77-2.88 (dt, J=21.74 Hz, 1H), 1.90-2.00 (m, 2H), 2.07-2.16 (m, 1H), 1.77-1.83 (m, 1H). ¹³C NMR (75 MHz, D₂O) a 167.89 (d, J=4.16 Hz, 1C), 164.48 (s, 1C), 137.00-137.13 (dd, J=7.12 Hz, 1C), 134.52 (s, 1C), 130.95 (d, J=8.86 Hz, 2C), 129.90 (s, 2C), 129.15 (s, 1C), 128.73 (s, 2C), 114.89 (d, J=21.90 Hz, 2C), 75.51 (s, 1C), 50.09 (s, 1C), 42.95-44.41 (d, J=110.78 Hz, 1C), 22.31 (d, J=4.00 Hz, 1C), 21.59 (d, J=8.00 Hz, 1C), 21.34 (s, 1C). ³¹P NMR (121 MHz, D₂O) δ 20.78. ¹⁹F NMR (282 MHz, D₂O) δ−113.20.

### Synthesis of P-(1-(benzyloxy)-2-oxopiperidin-3-yl)-N-(2,4-difluorobenzyl)phosphonamidic acid (7)

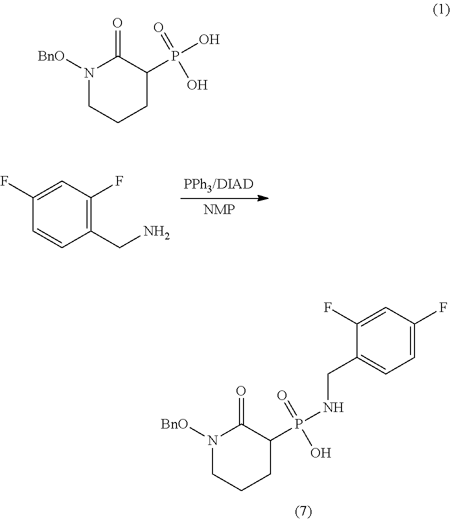 [SMILES: O=C1C(P(=O)(O)O)CCCN1OCc1ccccc1 ; NCc1ccc(F)cc1F ; O=C1C(P(=O)(O)NCc2ccc(F)cc2F)CCCN1OCc1ccccc1]

Following the general procedure above, the following quantities were used: DIAD (96.64 μL, 490.82 μmol) and triphenylphosphine (128.74 mg, 490.82 μmol) in anhydrous DCM (3 mL). BnHEX (1, 70 mg, 245.41 μmol) and (2,4-difluorophenyl)methanamine (58.55 μL, 490.82 μmol) in anhydrous DCM (1 mL). Yield: 61%. Analysis by ESI+(Expected [M+H]+=411.12. Observed [M+H]+=411.38). ¹H NMR (300 MHz, CDCl₃) δ 6.68-7.71 (m, 9H), 4.84 (m, 2H), 4.09 (s, 2H), 3.29 (m, 2H), 2.65 (m, 1H), 2.01 (m, 2H). 1.55-1.76 (2H, m); ¹³C NMR (75 MHz, CDCl₃) δ 176.91 (s, 1C), 158.62-164.51 (dd, J1=11.92 Hz, J2=150.9 Hz, 2C), 166.95 (d, J=4.42 Hz, 1C), 135.37 (s, 1C), 132.21 (s, 1C), 130.48 (dd, J1=9.60 Hz, J2=150.28 Hz, 1C), 129.31 (s, 2C), 128.59 (s, 1C), 128.41 (s, 2C), 124.55-124.88 (d, J=3.75 Hz, 1C), 118.25 (dq, J1=3.52 Hz, J2=14.70 Hz, 1C), 111.71 (dq, J1=3.52 Hz, J2=21.00 Hz, 1C), 103.44 (q, J1=25.12 Hz, 1C), 75.41 (s, 1C), 50.49 (s, 1C), 44.78-45.56 (d, J1=112.50 Hz, 1C), 39.14 (d, J=3.50 Hz, 1C), 36.06 (d, J=3.50 Hz, 1C), 23.19 (s, 1C), 22.35 (d, J=7.57 Hz, 1C). ³¹P NMR (121 MHz, CDCl₃) δ 18.98. ¹⁹F NMR (282 MHz, CDCl₃) −109.43 (d, J=7.86, 1F), 6-112.95 (d, J=6.86, 1F).

### Synthesis of P-(1-(benzyloxy)-2-oxopiperidin-3-yl)-N-(3,4-difluorobenzyl)phosphonamidic acid (8)

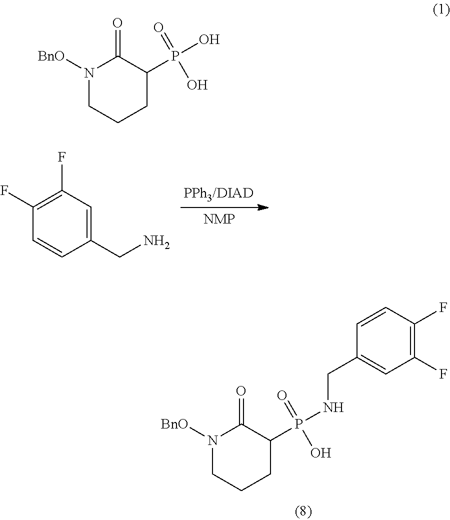 [SMILES: O=C1C(P(=O)(O)O)CCCN1OCc1ccccc1 ; NCc1ccc(F)c(F)c1 ; O=C1C(P(=O)(O)NCc2ccc(F)c(F)c2)CCCN1OCc1ccccc1]

Following the general procedure above, the following quantities were used: DIAD (276.11 μL, 1.40 mmol) and triphenylphosphine (367.82 mg, 1.40 mmol) in anhydrous DCM (3 mL). BnHEX (1, 200 mg, 245.41 μmol) and (3,4-difluorophenyl)methanamine (165.89 μL, 1.40 mmol) in anhydrous DCM (1 mL). Yield: 52%. Analysis by ESI+(Expected [M+H]+=411.36. Observed [M+H]+=411.42). ¹H NMR (300 MHz, CDCl₃) δ 7.21-7.25 (m, 8H), 4.77 (s, 2H), 3.91 (s, 2H), 3.21-3.35 (m, 2H), 2.50-2.62 (m, 1H), 1.90-1.92 (m, 1H). 1.75-1.79 (m, 1H), 1.46-1.50 (1H, m); ¹³C NMR (75 MHz, CDCl₃) δ 166.90 (s, 1C), 151.56-151.85 (m, 1C), 148.27-148.55 (m, 1C), 131.54-131.65 (dd, J1=3.75 Hz, J2=5.25 Hz, 1C), 129.28 (s, 2C), 128.69 (s, 1C), 128.46 (s, 2C), 125.51 (dd, J1=3.75 Hz, J1=5.25 Hz, 1C), 118.33 (d, J1=17.25 Hz, 1C) 117.09 (d, J1=17.25 Hz, 1C), 75.41 (s, 1C), 50.39 (s, 1C), 44.05-45.55 (d, J1=112.5 Hz, 1C), 45.00 (s, 1C), 42.10 (s, 1C), 23.18 (s, 1C), 22.38 (d, J=8.25 Hz, 1C). ³¹P NMR (121 MHz, CDCl₃) δ 18.95. ¹⁹F NMR (282 MHz, D₂O) 6-137.06 (d, J=21.32 Hz, 1F), −138.22 (d, J=12.73, 1F).

### Synthesis of P-(1-(benzyloxy)-2-oxopiperidin-3-yl)-N-methyl-N-(2-(pyrrolidin-1-yl)ethyl phosphoramidic acid (9)

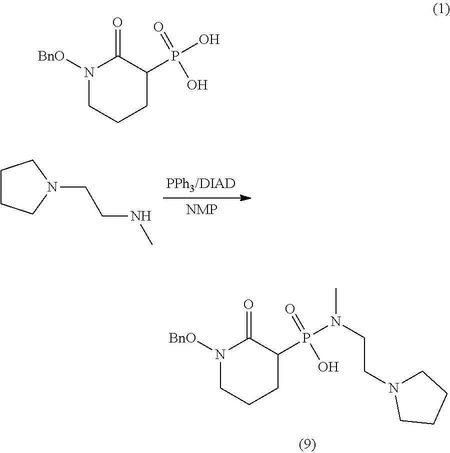

Following the general procedure above, the following quantities were used: DIAD (137.65 μL, 707.17 μmol) and triphenylphosphine (183.91 mg, 707.17 μmol) in anhydrous DCM (5 mL). BnHEX (1, 100 mg, 350.59 μmol) and N-methyl-2-(pyrrolidin-1-yl)ethan-1-amine (88.39 μL, 701.17 μmol) in anhydrous DCM (2 mL). Yield: 63%. Analysis by ESI+(Expected [M+H]+=396.44. Observed [M+H]+=396.51). ¹H NMR (300 MHz, D₂O) δ 7.47-7.54 (m, 5H), 4.99 (s, 2H), 3.59-3.63 (m, 2H), 2.99-3.04 (t, J=6.44, 7.03 Hz, 4H), 2.76-2.80 (dt, J=10.9 Hz, 1H), 2.63 (s, 3H), 2.00-2.25 (m, 7H), 1.90-1.97 (m, 4H), 1.82-1.90 (m, 1H). ¹³C NMR (75 MHz, D₂O) δ 167.92 (d, J=4.57 Hz, 1C), 134.40 (s, 1C), 129.86 (s, 2C), 129.20 (s, 1C), 128.76 (s, 2C), 75.45 (s, 1C), 53.86 (s, 2C), 51.46 (s, 1C), 49.91-50.10 (d, J=15.22 Hz, 1C), 46.20 (s, 1C), 41.40-42.88 (d, J=111.54 Hz, 1C), 33.20-33.25 (d, J=4.18 Hz, 1C), 22.55-22.62 (d, J=5.52 Hz, 1C), 22.29-22.35 (d, J=4.28 Hz, 1C), 21.07 (s, 2C). ³¹P NMR (121 MHz, D₂O) δ 22.00.

### Synthesis of P-(1-(benzyloxy)-2-oxopiperidin-3-yl)-N-(prop-2-yn-1-yl)phosphonamidic acid (10)

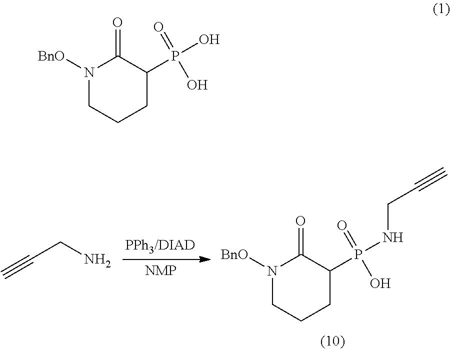

Following the general procedure above, the following quantities were used: DIAD (68.17 μL, 350.59 μmol) and triphenylphosphine (43.87 mg, 350.59 μmol) in anhydrous DCM (3 mL). BnHEX (1, 50 mg, 175.29 μmol) and propargylamine (16.84 μL, 262.94 μmol) in anhydrous DCM (1 mL). Yield: 35%. Analysis by ESI+(Expected [M+H]+=323.30. Observed [M+H]+=323.39). ¹H NMR (300 MHz, D₂O) δ 7.37-7.44 (m, 5H), 4.88 (s, 2H), 3.57-3.61 (dd, J=10.85 Hz, 2H), 3.43-3.49 (m, 2H), 2.84-2.96 (dt, J=22.10 Hz, 1H), 1.97-2.03 (m, 1H), 1.86-1.93 (m, 2H), 1.68-1.76 (m, 1H). ¹³C NMR (75 MHz, D₂O) δ 167.64 (d, J=4.50 Hz, 1C), 134.50 (s, 1C), 129.95 (s, 2C), 129.19 (s, 1C), 128.76 (s, 2C), 76.44 (s, 1C), 50.05 (s, 1C), 43.09-44.57 (d, 111.27 Hz, 1C), 30.07 (s, 1C), 22.24-22.29 (d, J=3.79 Hz, 1C), 21.69-21.79 (d, J=8.03 Hz, 1C); some tertiary and quaternary carbons not visible under these running conditions. ³¹P NMR (121 MHz, D₂O) δ 20.89.

### Synthesis of P-(1-(benzyloxy)-2-oxopiperidin-3-yl)-N-(4-fluorophenyl)phosphonamidic acid (11)

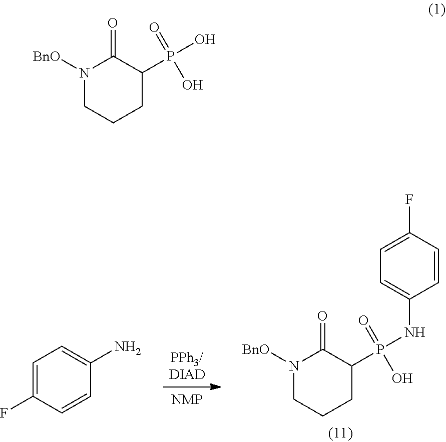

Following the general procedure above, the following quantities were used: DIAD (68.17 μL, 350.59 μmol) and triphenylphosphine (43.87 mg, 350.59 μmol) in anhydrous DCM (3 mL). BnHEX (1, 50 mg, 175.29 μmol) and 4-fluoroaniline (38.96 mg, 262.94 μmol) in anhydrous DCM (1 mL). Yield: 48%. Analysis by ESI+(Expected [M+H]+=379.34. Observed [M+H]+=379.38). ¹H NMR (300 MHz, D₂O) δ 7.46-7.54 (m, 5H), 7.41-7.44 (t, J=4.40, 4.61 Hz, 2H), 7.26-7.32 (t, J=8.67, 8.80 Hz, 2H), 4.98 (s, 2H), 3.53-3.57 (m, 2H), 2.85-2.97 (dt, J=24.41 Hz, 1H), 1.95-2.16 (m, 3H), 1.75-1.87 (m, 1H). ¹³C NMR (75 MHz, D₂O) δ 167.29-167.35 (d, J=5.19 Hz, 1C), 160.59-163.85 (d, J=246.61 Hz, 1C), 134.53 (s, 1C), 134.47 (s, 1C), 129.96 (s, 2C), 129.18 (s, 1C), 128.75 (s, 2C), 124.77-124.89 (d, J=9.77 Hz, 2C), 116.73-117.04 (d, J=23.62 Hz, 2C), 71.22 (s, 1C), 50.17 (s, 1C), 42.67-44.32 (d, J=124.63 Hz, 1C), 22.35-22.39 (d, J=3.65 Hz, 1C), 21.27-21.36 (d, J=7.25 Hz, 1C). ³¹P NMR (121 MHz, D₂O) δ 18.07. ¹⁹F NMR (282 MHz, D₂O) δ−113.00.

### Synthesis of N-(2-(aminomethyl)phenyl)-P-(1-(benzyloxy)-2-oxopiperidin-3-yl)phosphonamidic acid (12)

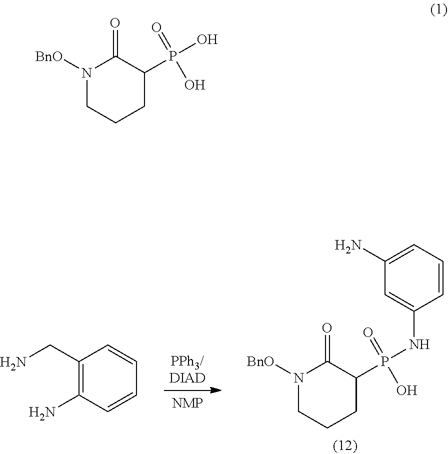

Following the general procedure above, the following quantities were used: DIAD (13.63 μL, 70.12 μmol) and triphenylphosphine (36.78 mg, 70.12 μmol) in anhydrous DCM (1.5 mL). BnHEX (1, 20 mg, 70.02 μmol) and 2-(aminomethyl)aniline (17.13 mg, 140.23 μmol) in anhydrous DCM (500 μL). Yield: 22%. Analysis by ESI+(Expected [M+H]+=390.39. Observed [M+H]+=390.39). ¹H NMR (300 MHz, D₂O) δ 7.44-7.53 (m, 5H), 7.17-7.22 (t, J=7.67, 7.78 Hz, 1H), 6.86-6.94 (m, 5H), 4.94 (s, 2H), 4.12 (s, 2H), 3.95 (d, J=6.80 Hz, 1H), 3.51-3.60 (m, 2H), 2.86-2.98 (dt, J=21.94 Hz, 1H), 2.08-2.16 (m, 1H), 1.95-2.04 (m, 2H), 1.75-1.83 (m, 1H). ¹³C NMR (75 MHz, D₂O) δ 167.87 (d, J=4.43 Hz, 1C), 144.75 (s, 1C), 134.52 (s, 1C), 130.39 (s, 2C), 129.90 (d, J=1.97 Hz, 1C), 129.62 (s, 1C), 129.15 (s, 1C), 128.72 (s, 2C), 128.56 (s, 1C), 118.79 (s, 1C), 117.01 (s, 1C), 75.64 (d, J=2.53 Hz, 1C), 43.94-42.49 (d, J=109.26 Hz, 1C), 42.60 (s, 1C), 22.25 (d, J=3.99 Hz, 1C), 21.56 (d, J=7.42 Hz, 1C), 21.32 (s, 1C). ³¹P NMR (121 MHz, D₂O) δ 21.15.

### Synthesis of P-(1-(benzyloxy)-2-oxopiperidin-3-yl)-N-(3-hydroxybenzyl)phosphonamidic acid (13)

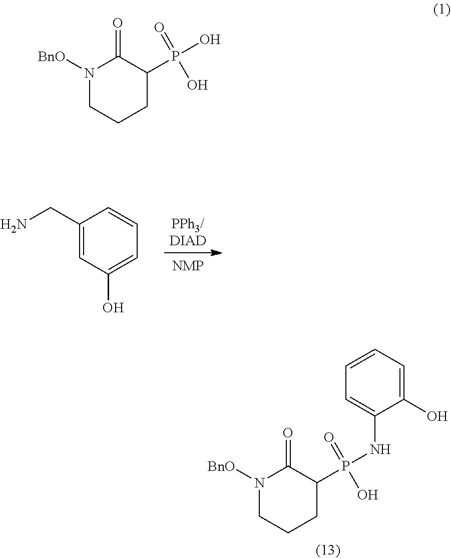

Following the general procedure above, the following quantities were used: DIAD (13.63 μL, 70.12 μmol) and triphenylphosphine (36.78 mg, 70.12 μmol) in anhydrous DCM (1.5 mL). BnHEX (1, 20 mg, 70.02 μmol) and 3-(aminomethyl)phenol (17.27 mg, 140.23 μmol) in anhydrous DCM (500 μL). Yield: 42%. Analysis by ESI+(Expected [M+H]+=391.38. Observed [M+H]+=391.37). ¹H NMR (300 MHz, D₂O) δ 7.44-7.52 (m, 6H), 7.26-7.35 (m, 3H), 4.92 (d, J=1.80 Hz, 2H), 3.99 (dd, J=8.48, 2H), 3.48-3.55 (m, 2H), 2.76-2.87 (dt, J=21.81 Hz, 1H), 2.04-2.14 (m, 1H), 1.89-1.99 (m, 2H), 1.71-1.81 (m, 1H). ¹³C NMR (75 MHz, D₂O) δ 167.90 (d, J=4.32 Hz, 1C), 156.57 (s, 1C), 143.34 (d, J=6.97 Hz, 1C), 134.51 (s, 1C), 130.53 (s, 2C), 129.89 (s, 1C), 129.14 (s, 1C), 128.71 (s, 2C), 116.36 (s, 1C), 115.92 (s, 1C), 119.59 (s, 1C), 75.51 (s, 1C), 43.05-45.07 (d, J=153.89 Hz, 1C), 44.51 (s, 1C), 22.34 (d, J=3.96 Hz, 1C), 21.60 (d, J=7.81 Hz, 1C), 21.27 (s, 1C). ³¹P NMR (121 MHz, D₂O) δ 23.29.

Synthesis of phosphoramidates from phosphates. For phosphate-containing compounds (1-naphthyl phosphate, Fludarabine monophosphate), reagents were added in the following order: 1.) phosphate starting material, 2.) triphenyl phosphine (2 equiv.), 3.) amine (2 equiv.), 4.) DIAD (2 equiv.). After 1 second, the reaction was transferred to a 5 mL Eppendorf tube and chloroform (2.5 mL), followed by water (2.5 mL) were added to the reaction. The reaction was vortexed and centrifuged (4000 rpm, 4° C.) for 2 minutes. The aqueous layer was then isolated and lyophilized to a clear oil.

### ADDITIONAL EXAMPLES

Using procedures similar to those above with the addition of 2 equivalents of DBU and with the following amines, the correspond compounds of Formula I were prepared.

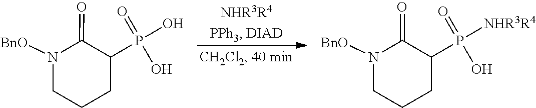

| Amine | % Yield^{§} |
|---|---|
| Cyclopropylmethanamine | 95 |
| Cyclobutanamine | 85 |
| Cyclohexane methylamine | 83 |
| Dodecylamine | 80 |
| Dimethylamine | 86 |
| Methylamine | 97 |

^{§}Yields were calculated via ³¹P NMR spectroscopic analysis of the crude reaction mixture, using TPPO as an internal standard.

### Synthesis of naphthalen-1-yl hydrogen benzylphosphoramidate (15)

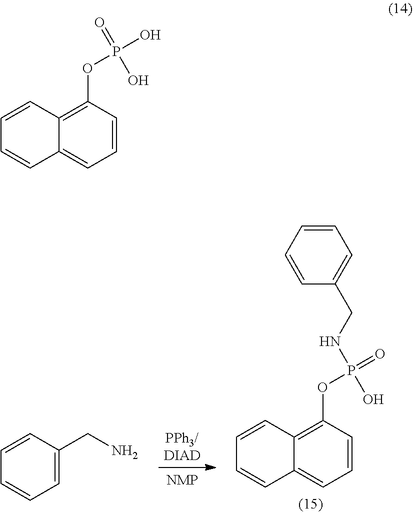

Following the general procedure above, the following quantities were used: 1-naphthyl phosphate (5 mg, 22.31 μmol, 14), benzylamine (4.87 μL, 44.61 μmol), triphenylphosphine (11.70 mg, 44.61 μmol), and DIAD (8.67 μL, 44.61 μmol) in anhydrous NMP (500 μL). Yield: 70%. Analysis by ESI+(Expected [M+H]+=314.29. Observed [M+H]+=314.32). ¹H NMR (300 MHz, D₂O) δ 8.10-8.13 (d, J=7.05 Hz, 1H), 7.95 (d, J=6.77 Hz, 1H), 7.70-7.73 (d, J=8.54 Hz, 1H), 7.55-7.59 (m, 2H), 7.46-7.55 (t, J=7.79, 7.79 Hz, 1H), 7.38-7.41 (d, J=7.79 Hz, 1H), 7.22-7.28 (m, 5H), 4.05 (d, J=10.73 Hz, 2H). ¹³C NMR (125 MHz, D₂O) δ 148.13 (d, J=7.70 Hz, 1C), 140.79 (d, J=6.73 Hz, 1C), 134.50 (s, 1C), 129.98 (s, 1C), 128.38 (s, 2C), 127.58 (s, 1C), 127.30 (s, 2C), 126.83 (s, 2C), 126.56 (s, 1C), 126.00 (d, J=3.70 Hz, 1C), 123.18 (s, 1C), 122.06 (s, 1C), 114.62 (d, J=3.68 Hz, 1C), 45.34 (s, 1C). ³¹P NMR (121 MHz, D₂O) δ 5.11.

### Synthesis of naphthalen-1-yl hydrogen (pyridin-2-ylmethyl)phosphoramidate (16)

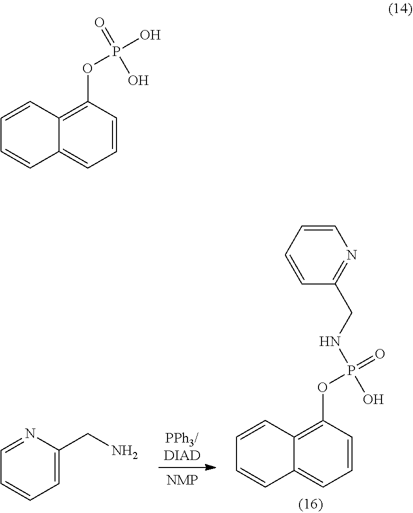

Following the general procedure above, the following quantities were used: 1-naphthyl phosphate (5 mg, 22.31 μmol, 14), pyridin-2-ylmethanamine (4.60 μL, 44.61 μmol), triphenylphosphine (11.70 mg, 44.61 μmol), and DIAD (8.67 μL, 44.61 μmol) in anhydrous NMP (500 μL). Yield: 72%. Analysis by ESI+(Expected [M+H]+=315.28. Observed [M+H]+=315.22). ¹H NMR (300 MHz, D₂O) δ 8.56 (d, J=5.07 Hz, 1H), 8.10 (d, J=5.15 Hz, 1H), 7.96-8.00 (d, J=8.67 Hz, 1H), 7.87-7.92 (t, J=7.82, 7.97 Hz, 1H), 7.81-7.84 (d, J=7.76 Hz, 1H), 7.59-7.62 (d, J=7.82 Hz, 1H), 7.35-7.52 (m, 4H), 7.13-7.15 (d, J=7.87 Hz, 1H), 4.11-4.15 (d, J=12.88 Hz, 2H). ¹³C NMR (125 MHz, D₂O) δ 158.38 (d, J=4.91 Hz, 1C), 149.18 (s, 1C), 147.88 (d, J=7.49 Hz, 1C), 138.40 (s, 1C), 134.34 (s, 1C), 127.52 (s, 1C), 126.48 (s, 2C), 125.86 (d, J=1.37 Hz, 1C), 124.12 (s, 1C), 123.16 (d, J=0.89 Hz, 1C), 122.36 (s, 1C), 121.89 (s, 1C), 114.26 (d, J=3.48 Hz, 1C), 45.92 (s, 1C). ³¹P NMR (121 MHz, D₂O) δ 4.59.

### Synthesis of naphthalen-1-yl hydrogen (4-fluorobenzyl)phosphoramidate (17)

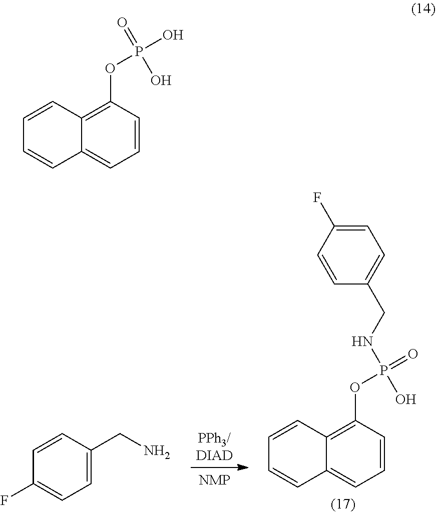

Following the general procedure above, the following quantities were used: 1-naphthyl phosphate (5 mg, 22.31 μmol, 14), 4-fluorobenzylamine (5.10 μL, 44.61 μmol), triphenylphosphine (11.70 mg, 44.61 μmol), and DIAD (8.67 μL, 44.61 μmol) in anhydrous NMP (500 μL). Yield: 68%. Analysis by ESI+(Expected [M+H]+=332.28. Observed [M+H]+=332.29). ¹H NMR (300 MHz, D₂O) δ 8.06-8.09 (d, J=7.57 Hz, 1H), 7.91-7.94 (d, J=8.43 Hz, 1H), 7.68-7.70 (d, J=8.43 Hz, 1H), 7.44-7.58 (m, 3H), 7.12-7.25 (m, 4H), 6.87-6.90 (d, J=8.89 Hz, 1H), 3.99-4.03 (d, J=11.55 Hz, 2H). ¹³C NMR (125 MHz, D₂O) δ 128.86-131.03 (dd, J=8.95, 9.04, 265.33 Hz, 2C), 127.52 (s, 1C), 126.52 (s, 1C), 125.92-125.5 (d, J=3.47 Hz, 1C), 123.08 (d, J=1.56 Hz, 1C), 122.03 (s, 1C), 114.60-116.03 (dd, J=162.43 Hz, 2C), 30.72-30.84 (d, J=15.39 Hz, 1C); quaternary carbons and some tertiary carbons not visible under these running conditions. ³¹P NMR (121 MHz, D₂O) δ 5.02. ¹⁹F NMR (282 MHz, D₂O) δ−113.24.

### Synthesis of naphthalen-1-yl hydrogen (2,6-difluorobenzyl)phosphoramidate (18)

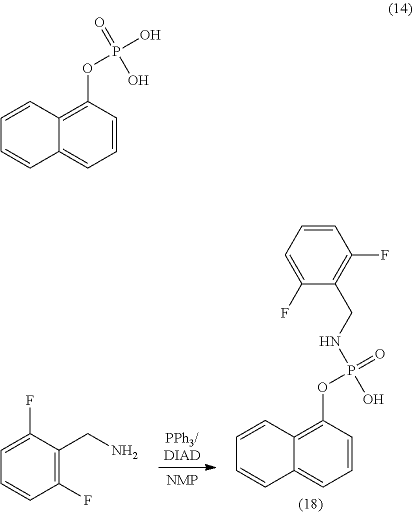

Following the general procedure above, the following quantities were used: 1-naphthyl phosphate (5 mg, 22.31 μmol, 14), (2,6-difluorophenyl)methanamine (5.33 μL, 44.61 μmol), triphenylphosphine (11.70 mg, 44.61 μmol), and DIAD (8.67 μL, 44.61 μmol) in anhydrous NMP (500 μL). Yield: 34%. Analysis by ESI+(Expected [M+H]+=350.27. Observed [M+H]+=350.25). ¹H NMR (300 MHz, D₂O) δ 8.25-8.27 (d, J=6.64 Hz, 1H), 7.96-7.99 (d, J=7.01 Hz, 1H), 7.83-7.90 (m, 1H), 7.74-7.77 (d, J=8.23 Hz, 1H), 7.60-7.74 (p, J=2.10, 2.29, 3.84, 3.84 Hz, 2H), 7.49-7.51 (t, J=3.21, 3.65 Hz, 1H), 7.42-7.45 (d, J=7.64 Hz, 1H), 7.08-7.13 (t, J=8.02, 8.11 Hz, 2H), 4.13-4.18 (d, J=13.24 Hz, 2H). ¹³C NMR (125 MHz, D₂O) δ 160.09-162.12 (dd, J=7.28, 246.98 Hz, 2C), 147.94 (d, J=7.15 Hz, 1C), 134.45 (s, 1C), 127.64 (s, 2C), 126.88 (s, 2C), 126.23 (s, 1C), 126.02 (d, J=1.52 Hz, 1C), 123.71 (d, J=1.32 Hz, 1C), 122.03 (s, 1C), 115.08 (d, J=3.20 Hz, 1C), 111.79-111.60 (dd, J=5.00, 5.00, 19.23 Hz, 1C), 33.04 (t, J=3.56, 3.78 Hz, 1C). ³¹P NMR (121 MHz, D₂O) δ 3.99. ¹⁹F NMR (282 MHz, D₂O) δ−114.99.

### Synthesis of naphthalen-1-yl hydrogen (cyclohexylmethyl)phosphoramidate (19)

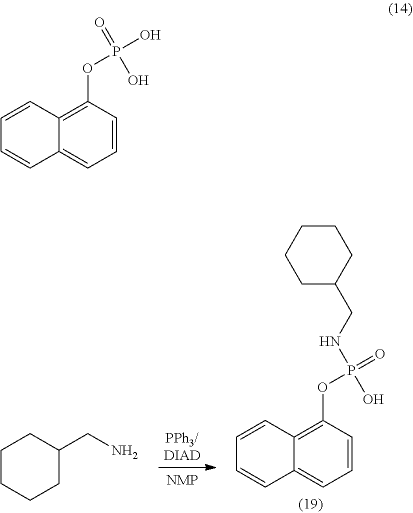

Following the general procedure above, the following quantities were used: 1-naphthyl phosphate (5 mg, 22.31 μmol, 14), cyclohexane methylamine (5.80 μL, 44.61 μmol), triphenylphosphine (11.70 mg, 44.61 μmol), and DIAD (8.67 μL, 44.61 μmol) in anhydrous NMP (500 μL). Yield: 86%. Analysis by ESI+(Expected [M+H]+=320.34. Observed [M+H]+=320.33). ¹H NMR (300 MHz, D₂O) δ 8.33 (d, J=4.52 Hz, 1H), 7.98 (d, J=4.96 Hz, 1H), 7.71-7.73 (d, J=8.16 Hz, 1H), 7.60-7.63 (m, 2H), 7.50-7.55 (t, J=7.81, 7.88 Hz, 1H), 7.44-7.45 (d, J=7.53 Hz, 1H), 2.61-2.66 (dd, J=10.82 Hz, 2H), 1.62-1.76 (m, 11H). ¹³C NMR (125 MHz, D₂O) 148.73 (d, J=7.40 Hz, 1C), 134.41 (s, 1C), 127.54 (s, 1C), 126.17 (d, J=1.15 Hz, 1C) 126.06 (s, 1C), 125.95 (s, 2C), 122.35 (s, 1C), 114.72 (d, J=3.11 Hz, 1C), 51.93 (s, 1C), 37.79 (s, 1C), 25.49 (s, 1C), 24.95 (s, 2C), 21.05 (s, 2C); some quaternary carbons not visible under these running conditions. ³¹P NMR (121 MHz, D₂O) δ 6.27.

### Synthesis of naphthalen-1-yl hydrogen cyclopropylphosphoramidate (20)

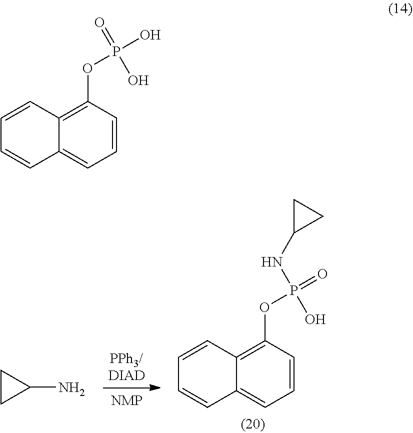

Following the general procedure above, the following quantities were used: 1-naphthyl phosphate (5 mg, 22.31 μmol, 14), cyclopropylamine (3.09 μL, 44.61 μmol), triphenylphosphine (11.70 mg, 44.61 μmol), and DIAD (8.67 μL, 44.61 μmol) in anhydrous NMP (500 μL). Yield: 91%. Analysis by ESI+(Expected [M+H]+=264.23. Observed [M+H]+=264.21). ¹H NMR (300 MHz, D₂O) δ 8.31 (d, J=6.10 Hz, 1H), 7.97 (d, J=6.34 Hz, 1H), 7.73 (d, J=7.71 Hz, 1H), 7.61-7.64 (p, J=1.71, 1.76, 3.86, 3.89 Hz, 2H), 7.50-7.55 (t, J=7.84, 8.87 Hz, 1H), 7.44-7.47 (d, J=7.70 Hz, 1H), 2.3-2.45 (m, 1H), 0.85-0.88 (d, J=5.20 Hz, 2H), 0.51-0.54 (d, J=6.37 Hz, 2H). ¹³C NMR (125 MHz, D₂O) δ 148.90 (d, J=7.20 Hz, 1C), 134.44 (s, 1C), 127.63 (s, 1C), 126.66 (s, 1C), 126.12 (s, 2C), 126.06 (d, J=1.36 Hz, 1C), 123.77 (d, J=1.61 Hz, 1C), 122.15 (s, 1C), 114.62 (d, J=3.28 Hz, 1C), 6.10 (s, 2C), 6.02 (d, J=5.39 Hz, 1C). ³¹P NMR (121 MHz, D₂O) δ 5.0.

### Synthesis of isopropyl (hydroxy(naphthalen-1-yloxy)phosphoryl)-L-alaninate 21)

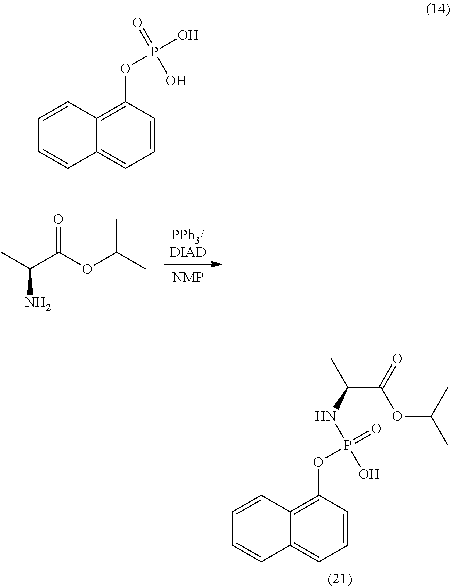

Following the general procedure above, the following quantities were used: 1-naphthyl phosphate (5 mg, 22.31 μmol, 14), cyclohexane methylamine (5.80 μL, 44.61 μmol), triphenylphosphine (11.70 mg, 44.61 μmol), and DIAD (8.67 μL, 44.61 μmol) in anhydrous NMP (500 μL). Yield: 86%. Analysis by ESI+(Expected [M+H]+=320.34. Observed [M+H]+=320.33). ¹H NMR (300 MHz, D₂O) δ 8.25-8.28 (d, J=9.76 Hz, 1H), 7.96-7.99 (d, J=9.39 Hz, 1H), 7.75-7.77 (d, J=8.82 Hz, 1H), 7.60-7.64 (m, 2H), 7.50-7.55 (t, J=7.39, 7.86 Hz, 1H), 7.43-7.46 (d, J=7.86 Hz, 1H), 5.07-5.15 (m, 1H), 3.80-3.85 (m, 1H), 1.54-1.57 (d, J=7.20 Hz, 6H), 1.24-1.27 (d, J=7.42 Hz, 3H). ¹³C NMR (125 MHz, D₂O) δ 176.27-176.30 (d, J=4.29 Hz, 1C), 147.95-147.89 (d, J=7.13 Hz, 1C), 134.46 (s, 1C), 127.66 (s, 1C), 126.71 (s, 2C), 126.25 (s, 1C), 126.04 (d, J=1.41 Hz, 1C), 123.73 (d, J=1.11 Hz, 1C), 122.04 (s, 1C), 115.07-115.09 (d, J=3.14 Hz, 1C), 70.22 (s, 1C), 55.83 (s, 1C), 20.26 (s, 2C), 15.04 (s, 1C). ³¹P NMR (121 MHz, D₂O) δ 2.66.

### Additional Compounds

Using procedures similar to those above with the addition of 2 equivalents of DBU and with the following amines, the correspond compounds of Formula I were prepared.

| Amine | % Yield^{§} |
|---|---|
| Oleylamine | 9 |
| (S)-1-phenylethan-1-amine | 97 |
| (R)-1-phenylethan-1-amine | 97 |
| D-(+)-Glucosamine* | 40 |

^{§}Yields were calculated via ³¹P NMR spectroscopic analysis of the crude reaction mixture, using TPPO as an internal standard.
Yields with asterisks indicate the addition of 2 equivalents of DBU to the reaction.

### Synthesis of ((2S,3R,4R,5R)-5-(6-amino-2-fluoro-9H-purin-9-yl)-3,4-dihydroxytetrahydrofuran-2-yl)methyl hydrogen benzylphosphoramidate (23)

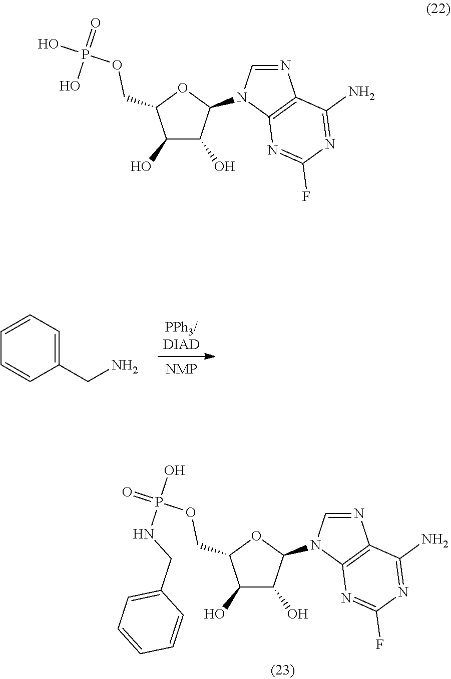

Following the general procedure above, the following quantities were used: Fludarabine monophosphate (5 mg, 13.69 μmol, 22), benzylamine (2.99 μL, 27.38 μmol), triphenylphosphine (7.18 mg, 27.38 μmol), and DIAD (5.38 μL, 27.38 μmol) in anhydrous NMP (500 μL). Yield: 52%. Analysis by ESI+(Expected [M+H]+=455.36. Observed [M+H]+=455.48). ¹H NMR (300 MHz, D₂O) δ 8.26 (s, 1H), 7.42-7.47 (m, 5H), 6.20-6.22 (d, J=6.12 Hz, 1H), 4.59-4.63 (t, J=6.47, 6.71 Hz, 1H), 4.42-4.46 (t, J=6.86, 6.86 Hz, 1H), 4.01-4.07 (m, 3H), 3.84-3.87 (d, J=9.65 Hz, 2H). ¹³C NMR (125 MHz, D₂O) δ 156.5-160.22 (d, J=255.64 Hz, 1C), 150.09-50.34 (d, J=18.85 Hz, 1C), 140.64-140.74 (d, J=7.41 Hz, 1C), 129.17 (s, 2C), 128.60 (s, 1C), 128.28 (s, 2C), 128.08 (s, 1C), 116.31-116.36 (d, J=4.16 Hz, 1C), 82.84 (s, 1C), 80.95-81.07 (d, J=8.88 Hz, 1C), 75.39 (s, 1C), 73.26 (s, 1C), 62.86-62.93 (d, J=5.17 Hz, 1C), 44.93 (s, 1C). ³¹P NMR (121 MHz, D₂O) δ 8.70.

### Synthesis of ((2S,3R,4R,5R)-5-(6-amino-2-fluoro-9H-purin-9-yl)-3,4-dihydroxytetrahydrofuran-2-yl)methyl hydrogen (pyridin-2-ylmethyl)phosphoramidate (24)

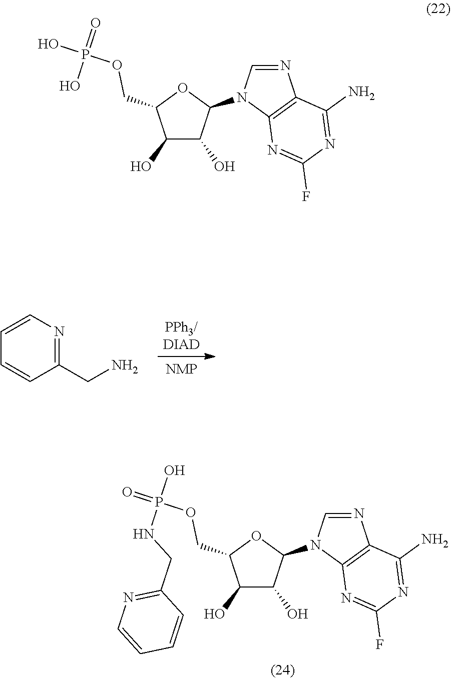

Following the general procedure above, the following quantities were used: Fludarabine (5 mg, 13.69 μmol, 22), pyridin-2-ylmethanamine (2.82 μL, 27.38 μmol), triphenylphosphine (7.18 mg, 27.38 μmol), and DIAD (5.38 μL, 27.38 μmol) in anhydrous NMP (500 μL). Yield: 69%. Analysis by ESI+(Expected [M+H]+=456.34. Observed [M+H]+=456.42). ¹H NMR (300 MHz, D₂O) δ 8.56-8.58 (d, J=4.88 Hz, 1H), 8.22 (s, 1H), 7.47-7.50 (d, J=8.38 Hz, 1H), 7.46-7.47 (t, J=3.70, 3.90 Hz, 1H), 7.16-7.20 (t, J=6.61, 6.63 Hz, 1H), 6.16-618 (d, J=6.29 Hz, 1H), 4.58-4.63 (t, J=6.49, 6.95 Hz, 1H), 4.42-4.45 (t, J=6.95, 6.95 Hz, 1H), 4.04-4.13 (m, 3H), 3.99-4.02 (d, J=10.18 Hz, 2H). ¹³C NMR (75 MHz, D₂O) δ 151.47-154.72 (d, J=246.11 Hz, 1C), 149.18 (s, 1C), 147.18 (s, 1C), 141.16-141.20 (d, J=2.47 Hz, 1C), 138.52 (s, 1C), 123.09 (s, 1C), 122.42 (s, 1C), 82.72 (s, 1C), 80.79-80.90 (d, J=8.60 Hz, 1C), 75.36 (s, 1C), 73.09 (s, 1C), 62.87-62.93 (d, J=4.82 Hz, 1C), 45.77 (s, 1C); some quaternary carbons not visible under these running conditions. ³¹P NMR (121 MHz, D₂O) δ 8.38.

### Synthesis of ((2S,3R,4R,5R)-5-(6-amino-2-fluoro-9H-purin-9-yl)-3,4-dihydroxytetrahydrofuran-2-yl)methyl hydrogen cyclopropylphosphoramidate (25)

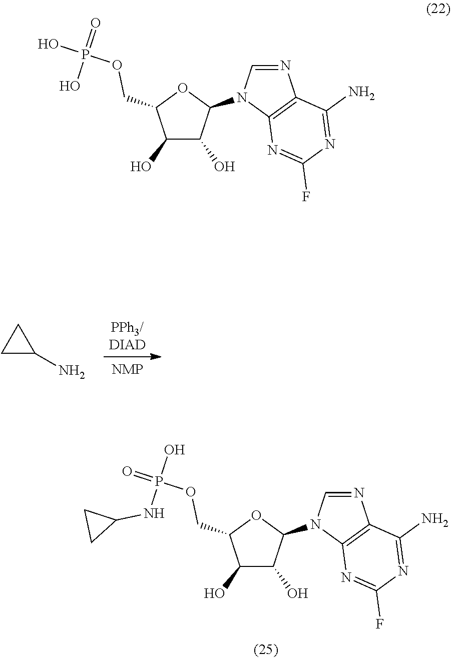

Following the general procedure above, the following quantities were used: Fludarabine monophosphate (5 mg, 13.69 μmol, 22), cyclopropylamine (1.90 μL, 27.38 μmol), triphenylphosphine (7.18 mg, 27.38 μmol), and DIAD (5.38 μL, 27.38 μmol) in anhydrous NMP (500 μL). Yield: 38%. Analysis by ESI+(Expected [M+H]+=405.30. Observed [M+H]+=405.37). ¹H NMR (300 MHz, D₂O) δ 8.44 (s, 1H), 6.31-6.33 (d, J=6.08 Hz, 1H), 4.61-4.65 (t, J=6.29, 6.33 Hz, 1H), 4.46-4.50 (t, J=6.90, 6.80 Hz, 1H), 4.14-422 (m, 3H), 2.24-2.32 (m, 1H), 0.86-0.89 (m, 2H), 0.44-0.467 (m, 2H). ¹³C NMR (75 MHz, D₂O) δ 149.25-149.43 (d, J=13.71 Hz, 1C), 141.07-141.10 (d, J=2.79 Hz, 1C), 83.40 (s, 1C), 80.09-81.11 (d, J=8.75 Hz, 1C), 75.51 (s, 1C), 73.16 (s, 1C), 63.15-63.18 (d, J=3.15 Hz, 1C), 22.92-23.00 (d, J=5.93 Hz, 1C), 6.11 (s, 2C); some quaternary carbons not visible under these running conditions. ³¹P NMR (121 MHz, D₂O) δ 7.97.

### Synthesis of isopropyl ((((2S,3R,4R,5R)-5-(6-amino-2-fluoro-9H-purin-9-yl)-3,4-dihydroxytetrahydrofuran-2-yl)methoxy)(hydroxy)phosphoryl)-D-alaninate (26)

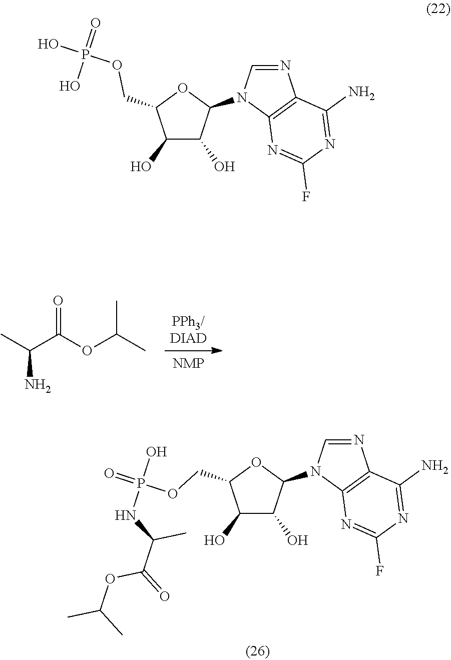

Following the general procedure above, the following quantities were used: Fludarabine monophosphate (5 mg, 13.69 μmol, 22), isopropyl L-alaninate hydrochloric acid salt (3.59 mg, 27.38 μmol), triethylamine (3.82 μL, 27.39 μmol), triphenylphosphine (7.18 mg, 27.38 μmol), and DIAD (5.38 μL, 27.38 μmol) in anhydrous NMP (500 μL). Yield: 36%. Analysis by ESI+(Expected [M+H]+=479.37. Observed [M+H]+=479.50). ¹H NMR (300 MHz, D₂O) δ 8.14 (s, 1H), 5.99-6.00 (d, J=6.37 Hz, 1H), 5.09-5.17 (m, 1H), 4.61-4.65 (t, J=6.55, 6.67 Hz, 1H), 4.30-4.35 (t, J=7.99, 7.99 Hz, 1H), 4.09-4.20 (m, 3H), 3.61-3.71 (m, 1H), 1.57-1.59 (d, J=7.34 Hz, 3H), 1.28-1.30 (d, J=7.78 Hz, 6H). ¹³C NMR (75 MHz, D₂O) δ 176.77-176.83 (d, J=4.58 Hz, 1C), 156.41-156.67 (d, J=18.94 Hz, 1C), 140.33-140.36 (d, J=2.61 Hz, 1C), 85.24 (s, 1C), 83.22-83.29 (d, J=5.37 Hz, 1C), 75.65 (s, 1C), 73.59 (s, 1C), 48.94 (s, 1C), 72.30 (s, 1C), 63.26-63.32 (d, J=4.66 Hz, 1C), 20.64 (s, 2C), 15.07 (s, 1C); some quaternary carbons not visible under these running conditions. ³¹P NMR (121 MHz, D₂O) δ 6.37.

### Synthesis of P—((((S)-1-(6-amino-9H-purin-9-yl)propan-2-yl)oxy)methyl)-N-methylphosphonamidic acid (P-methylamido tenofovir) (28)

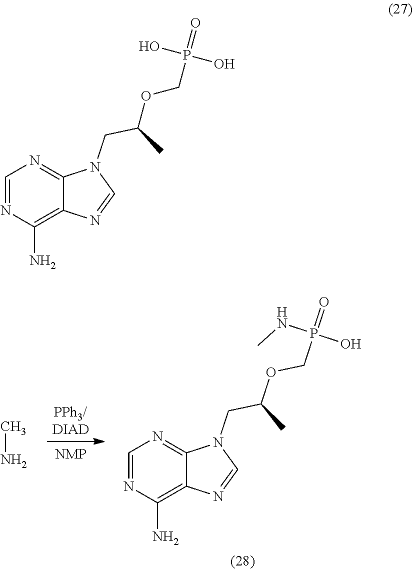

To a solution of triphenylphosphine (146.12 mg, 557.07 μmol) in anhydrous NMP (1.5 mL), diisopropyl (E)-diazene-1,2-dicarboxylate (108.31 μL, 557.07 μmol) was added at −78° C. under argon. The mixture was allowed to stir for 30 minutes. Separately, a solution of tenofovir (27) ((S)-(((1-(6-amino-9H-purin-9-yl)propan-2-yl)oxy)methyl)phosphonic acid, 20 mg, 69.63 μmol), methylamine (2.0 M in THF, 278.54 μL, 557.07 μmol) and DBU (83.15 μL, 557.07 μmol) was prepared in anhydrous NMP (1.5 mL); this was then added dropwise to the solution containing the betaine at −78° C. The reaction stirred under argon for 30 minutes. The crude reaction mixture was then transferred to a 50 mL Falcon tube, where 1 volume of water was added. The reaction was vortexed and centrifuged (4000 rpm, 4° C.) for 2 minutes. The aqueous layer was then isolated and lyophilized to a colorless gum. The product (28) was used without further purification. Analysis by ESI− (Expected [M−H]⁻=299.25. Observed [M−H]⁻=298.88). ¹H NMR (300 MHz, CD₃OD) δ 8.21 (s, 1H), 8.10 (s, 1H), 3.75 (m, 1H), 3.27-3.56 (m, 2H), 2.26 (d, J=11.10 Hz, 3H), 1.07 (d, J=6.60 Hz, 3H). ¹³C NMR (75 MHz, CD₃OD) δ 155.86 (s, 1C), 151.73 (s, 1C), 149.19 (s, 1C), 141.73 (s, 1C), 117.74 (s, 1C), 75.48 (s, 1C), 64.51 (s, 1C), 53.97 (s, 1C), 26.38 (s, 1C), 15.1 (s, 1C). ³¹P NMR (121 MHz, CD₃OD) δ 19.40.

### Optimization Studies

The general procedure above with the following quantities was used to prepare N-benzyl-P-(1-benzyloxy)-2-oxopiperidin-3-yl)phosphonamidic acid.

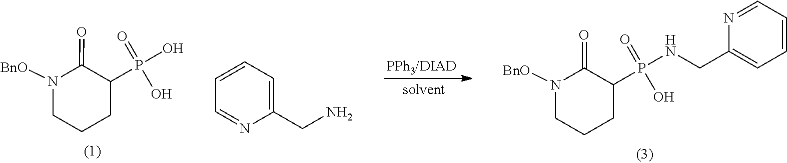 [SMILES: O=C1C(P(=O)(O)O)CCCN1OCc1ccccc1 ; NCc1ccccn1 ; O=C1C(P(=O)(O)NCc2ccccn2)CCCN1OCc1ccccc1]

| Solvent | Eq. Amine | Eq. PPh₃ | Eq. DIAD | Base | Yield (%)* |
|---|---|---|---|---|---|
| THF | 2 | 2 | 2 | None | n.d.^{†} |
| DMF | 2 | 2 | 2 | None | n.d.^{†} |
| MeCN | 2 | 2 | 2 | None | n.d.^{†} |
| DCM | 2 | 2 | 2 | None | 74 |
| DCM | 1 | 1 | 1 | TEA (2 eq.) | 20 |
| DCM | 1 | 1 | 1 | 2,6-lutidine | 17 |
| | | | | (2 eq.) | |
| Pyridine | 2 | 2 | 2 | None | n.d.^{†} |
| Toluene | 2 | 2 | 2 | None | n.d.^{†} |
| NMP | 2 | 2 | 2 | None | n.d.^{†} |
| CDCl₃ | 2 | 2 | 2 | None | 74 |
| CDCl₃ | 1 | 1 | 1 | None | n.d.^{†} |
| CDCl₃ | 2 | 1 | 1 | None | 62 |
| CDCl₃ | 1.5 | 1.5 | 1.5 | None | 64 |
| CDCl₃ | 1 | 1 | 1 | TEA (1 eq.) | 57 |
| CDCl₃ | 2 | 1 | 1 | DBU (1 eq.) | 65 |
| CDCl₃ | 2 | 1 | 1 | DBU (2 eq.) | 56 |

*Yields were calculated via³¹ P NMR spectroscopic analysis of the crude reaction mixture, using triphenylphosphine oxide as an internal standard.
^{†}Products were not detected by LCMS or the reaction resulted in phosphonate dimer formation, as indicated by a ³¹P NMR chemical shift at ~10 ppm in CDCl₃.

The general procedure above with the following quantities was used to prepare naphthalen-1-yl hydrogen benzylphosphoramidate.

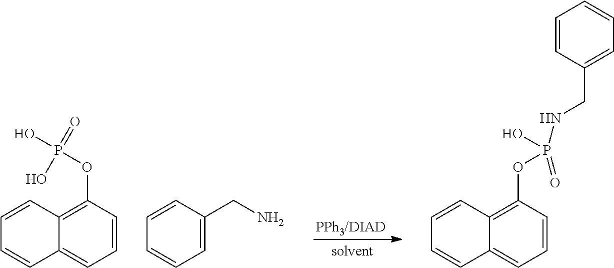

| Solvent | Eq. Amine | Eq. PPh₃ | Eq. DIAD | Base | Yield (%)* |
|---|---|---|---|---|---|
| NMP | 2 | 2 | 2 | None | 74 |
| NMP | 2 | 2 | 2 | DBU (2 eq.) | 70 |
| DMF | 2 | 2 | 2 | None | 74 |
| DMSO | 2 | 2 | 2 | None | 46 |
| Acetone | 2 | 2 | 2 | None | 20 |
| DCM (or | 2 | 2 | 2 | None | n.d.^{†} |
| CHCl₃) | | | | | |
| DCM (or | 2 | 2 | 2 | DBU (2 eq.) | 100 |
| CHCl₃) | | | | | |
| DCM (or | 2 | 1 | 1 | DBU (1 eq.) | 96 |
| CHCl₃) | | | | | |
| DCM (or | 1.5 | 1 | 1 | DBU (1 eq.) | 100 |
| CHCl₃) | | | | | |
| DCM (or | 1.5 | 1 | 1 | DBU (0.75 | 100 |
| CHCl₃) | | | | eq.) | |
| DCM (or | 1 | 1 | 1 | DBU (0.75 | 66 |
| CHCl₃) | | | | eq.) | |
| DCM (or | 1.5 | 1 | 1 | none | n.d.^{†} |
| CHCl₃) | | | | | |

*Yields were calculated via³¹ P NMR spectroscopic analysis of the crude reaction mixture, using triphenylphosphine oxide as an internal standard.
^{†}Reactants were insoluble.

### ADDITIONAL EXAMPLES

The following compounds were prepared using the procedures described below.

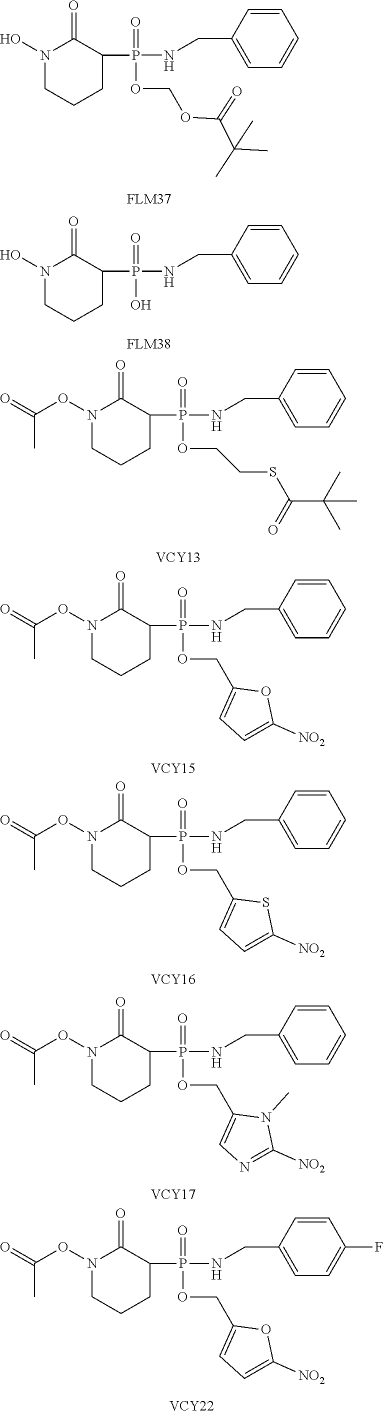

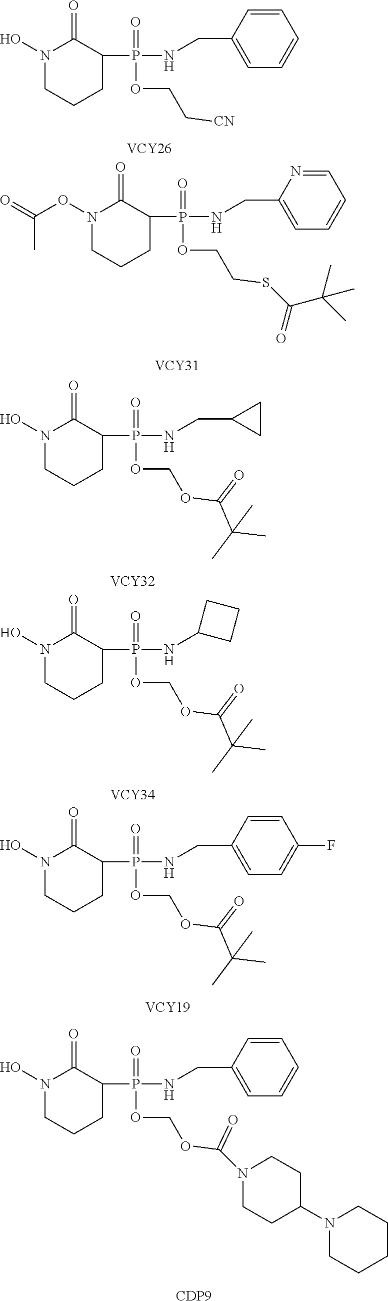

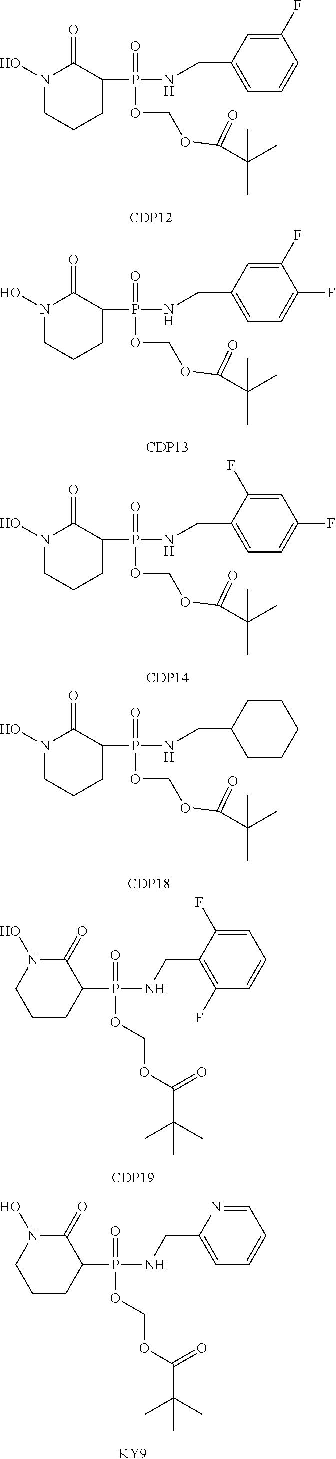

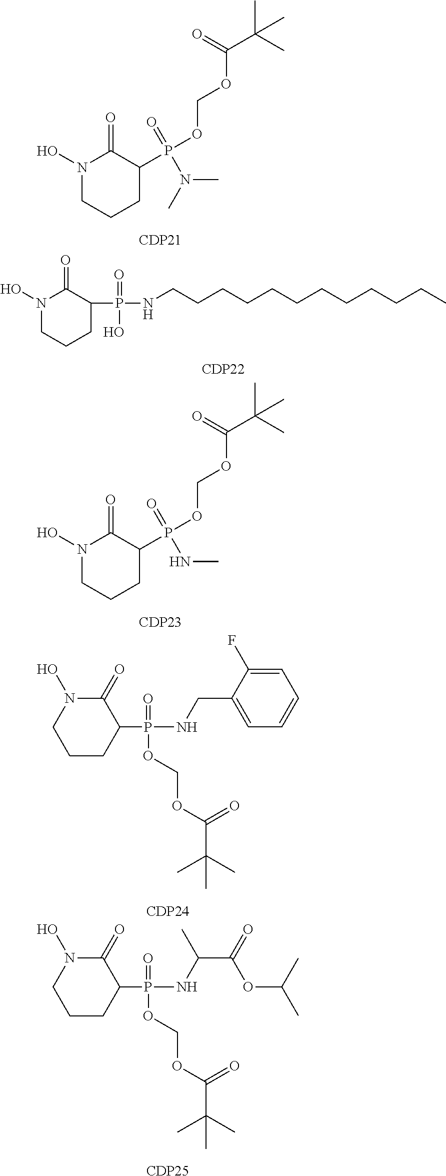

### Synthesis of FLM38 N-benzyl-P-(1-hydroxy-2-oxopiperidin-3-yl)phosphonamidic acid

A solution of 10%, Pd/C (200 mg) in anhydrous THF/MeOH (2:3) was stirred at 25° C. A balloon of H₂ was added and the solution vented for 10 minutes. A second balloon of H₂ was then added, and the solution stirred for 1 h. Then this slurry was transferred to a vial containing BnFLM38 (250 mg, 668 mmol) and was allowed to stir for 1 h. The reaction was filtered and concentrated to a yellow oil. Analysis by ESI+(Expected [M+H]+=285.25. Observed [M+H]⁺=285.23).

### Synthesis of BnFLM37 (((benzylamino)(1-(benzyloxy)-2-oxopiperidin-3-yl)phosphoryl)oxy)methyl pivalate

A mixture of BnFLM38 (118 mg, 315 mmol), triethylamine (120 μL, 0.901 mmol), and chloromethyl pivalate (336 μL, 3.14 mmol) in acetonitrile (60 mL) was stirred vigorously for 24 h at 60° C. This was then concentrated to yield a yellow oil. Next, the product was diluted in 118 mL of CHCl₃ and washed with 1 volume of 1 M HCl and then ddH₂O. The organic layer was removed and concentrated and lyophilized for 5 h to a yellow crystalline solid. Analysis by ESI+(Expected [M+H]⁺=489.52. Observed [M+H]⁺=489.42).

### Synthesis of FLM37 (((benzylamino)(1-hydroxy-2-oxopiperidin-3-yl)phosphoryl)oxy)methyl pivalate

A solution of 10% Pd/C (150 mg) in anhydrous THF/MeOH (2:3) was stirred at 25° C. A balloon of H₂ was added and the solution vented for 10 minutes. A second balloon of H₂ was then added, and the solution stirred for 1 h. Then, this slurry was transferred to a vial containing BnFLM37 (150 mg, 307 mmol) and was allowed to stir for 1 h. The reaction was filtered and concentrated to an orange solid. Analysis by ESI+(Expected [M+H]+=399.40. Observed [M+H]+=399.35).

### Synthesis of AcFLM38 P-(1-acetoxy-2-oxopiperidin-3-yl)-N-benzylphosphonamidic acid

To a solution FLM38 (65 mg, 229 μmol) in anhydrous MeCN (500 μL), Ac₂O (65 μL, 668 μmol) were added. The reaction stirred for 3 h at 25° C. Then, the reaction was concentrated and lyophilized for 2 days. Analysis by ESH. (Expected [M+H]⁺=327.29. Observed [M+H]⁺=327.32).

### Synthesis of VCY13, 3-((benzylamino)(2-(pivaloylthio)ethoxy)phosphoryl)-2-oxopiperidin-1-yl acetate

To a solution of AcFLM38 (15 mg, 46 mmol) in anhydrous CHCl₃, S-(2-hydroxyethyl)-2,2-dimethylpropanethioate (11.19 μL, 69 mmol) was added, followed by triphenylphosphine (18 mg, 69 mmol). Then DIAD (13.54 μL 69 mmol) was added. The reaction was allowed to stir at 25° C. for 20 h. The solvent was removed and the crude product was purified via reverse-phase HPLC. This was then lyophihzed to a yellow solid. Analysis by ESH. (Expected [[M+H]⁺=471.52. Observed [M+H]⁺=471.38, 471.42 (cis/trans isomers)). ³¹P NMR (121 MHz, CDCl₃) δ 28.87 (d, 1P).

### Synthesis of VCY15, 3-((benzylamino)((S-nitrofuran-2-yl)methoxy)phosphoryl)-2-oxopiperidin-1-yl acetate

To a solution of AcFLM38 (73 mg, 224 mmol) in anhydrous acetonitrile, 2-(bromornethyl)-5-nitrofuran (138.26 mg, 671 mmol) was added. The reaction was allowed to stir at 50° C. for 20 h. The solvent was then removed and the reaction was purified via reverse-phase HPLC. This was then lyophilized to a yellow solid. Analysis by ESI+(Expected [M+H]⁺=452.37. Observed [M+H]⁺=452.32).

### Synthesis of VCY16, 3-((benzylamino)((5-nitrothiophen-2-yl)methoxy)phosphoryl)-2-oxopiperidin-1-yi acetate

To a solution of AcFLM38 (6 mg, 19 mmol) in anhydrous CHCl₃, (5-nitrothiophen-2-y])methanol (4.39 mg, 28 mmol) was added, followed by triphenylphosphine (7.2 mg, 28 mmol). Then, DIAD (5.4 μL, 28 mmol) was added. The reaction was allowed to stir at 25° C. for 20 h. The solvent was removed and the crude product was purified via reverse-phase HPLC. This was then lyophillzed to an orange solid. Analysis by ESI+(Expected [M+H]⁺=468.43. Observed [M+H]⁺=468.32).

### Synthesis of VCY17, 3-((benzylamino)((1-methyl-2-nitro-1H-imidazol-5-yl)methoxy)phosphoryl)-2-oxopiperidin-1-yl acetate

To a solution of AcFLM38 (46 mg, 141 mmol) in anhydrous CHCl₃, (1-methyl-2-nitro-1H-imidazol-5-yl)methanol (33 mg, 211 mmol) was added, followed by triphenylphosphine (55 mg, 211 mmol). Then, DIAD (42 μL, 211 mmol) was added. The reaction was allowed to stir at 25° C. for 20 h. The solvent was removed and the crude product was purified via reverse-phase HPLC. This was then lyophilized to a yellow solid. Analysis by ESI+(Expected [M+H]⁺=466.40. Observed [M+H]⁺=466.34).

### General Synthetic Procedures

STEP 1-Synthesis of phosphonoamidates from BnHEX. All coupling reactions described for BnHEX (1) follow the same general procedure. Betaine formation: DIAD (2 equiv.) and triphenyl phosphine (2 equiv.) were combined in anhydrous DCM at 0° C. and allowed to stir to room temperature for 30 minutes. Separately, BnHEX (1 equiv.) and the indicated amine (2 equiv.) were dissolved in anhydrous DCM and then added dropwise to the betaine solution. The reaction was allowed to stir for 30 minutes. The crude reaction mixture was then transferred to a 50 mL Falcon tube, where 1 volume of water was added. The reaction was vortexed and centrifuged (4000 rpm, 4° C.) for 2 minutes The aqueous layer was then isolated and lyophilized to a white powder, unless otherwise specified.

### For Thioester and Nitroheterocycle Compounds

STEP 2. A mixture of palladium on carbon (10 wt %, 1 equiv. by mass) in THF/MeOH (2:3 ratio) was flushed with two H₂-containing balloons for 1 h. Separately, the compound obtained from STEP 1 was dissolved in MeOH and added to the palladium-containing slurry and allowed to react at room temperature for 12 h. The reaction was then filtered and concentrated under reduced pressure to a yellow oil. The product was used without further purification.

### For Pivaloyloxymethyl (POM) Compounds

STEP 2 To a solution of compound in STEP 1 in MeCN, POMCI (1.5 equiv.) and DIPEA (0.1 equiv.) were added. The reaction was allowed to stir for 15 h at 60° C. Then, the crude mixture was concentrated to a yellow oil and lyophilized for 15 h to a yellow oil. The crude product was used without further purification.

### For Thioester and Nitroheterocycle Compounds

STEP 3. To a solution of compound in STEP 2 in anhydrous MeCN, acetic anhydride (1.5 equiv.) and DIPEA (0.1 equiv.) were added. The reaction was stirred at 50° C. for 1 h. The crude mixture was then concentrated to a yellow oil and used without further purification.

### For Pivaloyloxymethyl (POM) Compounds

STEP 3. A mixture of palladium on carbon (10 wt. %, 1 equiv. by mass) in THF/MeOH (2:3 ratio) was flushed with two H₂-containing balloons for 1 h. Separately, the compound obtained from STEP 2 was dissolved in MeOH and added to the palladium-containing slurry and allowed to react at room temperature for 12 h. The reaction was then filtered and concentrated under reduced pressure to a yellow oil. Then, the crude product was purified via reverse-phase HPLC (Aligent G1361 A 1260 Infinity) using a stepwise gradient (5-90%) Buffer B over 10 minutes, 90-100% Buffer B over 7 minutes, 100% Buffer B over 8 minutes, 100-5%, Buffer B over 5 minutes: Buffer A: dH₂O with 0.1% TFA, Buffer B: CH₃CN+0.1% TFA). Product-containing fractions were combined and lyophilized to a pale yellow oil.

### For Thioester and Nitroimidazole Compounds

STEP 4. To a solution of compound in STEP 3 in anhydrous DCM, S-(2-hydroxyethyl) 2,2-dimethylpropanethioate, OR (1-methyl-2-nitro-1H-imidazol-5-yl)methanol (2 equiv.), and DIAD (2 equiv.) were added sequentially. The reaction—was allowed to react with end-over-end rotation for 15 h. Next, the crude reaction mixture was concentrated to a yellow-orange oil. Then, the crude product was purified via reverse-phase HPLC (Aligent G 1361 A 1260 Infinity) using a stepise gradient (5-90% Buffer B over 10 minutes, 90-100% Buffer B over 7 minutes, 100% Buffer B over 8 minutes, 100-5% Buffer B over 5 minutes; Buffer A: dH₂O with 0.1% TFA, Buffer B: CH₃CN+0.1% TFA). Product-containing fractions were combined and lyophilized to a pale-yellow powder.

### For Nitroheterocycle Compounds

STEP 4. To a solution of compound in STEP 3 in anhydrous:MeCN. 2-(bromomethyl)-5-nitrofuran, OR 2-(bromomethyl)-5-nitrothiophene (1.5 equiv), followed by TEA (02 equiv.) were added. The reaction was allowed to stir at 50° C. for 12 h. The reaction was then concentrated under reduced pressure and purified via reverse-phase HPLC (Aligent G 1361A 1260 Infinity) using a stepwise gradient (5-90% Butler B over 10 minutes, 90-100′% Buffer B over 7 minutes, 100% Buffer B over 8 minutes, 100-5% Buffer B over 5 minutes: Buffer A: dH₂O with 0.1% TFA, Buffer B: CH₃CN+0.1% TFA). Product-containing fractions were combined and lyophilized to an orange oil.

### For VCY26

STEP 1. N-benzyl-P-(1-(benzyloxy)-2-oxopiperidin-3-yl)phosphonamidic acid (25 mg, 87.65 μmol) was dissolved in neat phosphorous oxychloride (100 μL) and allowed to react with end-over-end rotation for 40 minutes. Then, the crude reaction mix was dissolved in chloroform (5 mL) and the reaction was washed first with 1 volume of water, followed by 1 volume of saturated sodium bicarbonate, 1 volume of water, 1 volume of brine, and then 1 volume of water. The organic layer was dried over sodium sulfate and concentrated under reduced pressure to a pale-yellow oil.

STEP 2. To a solution of compound obtained in STEP 1 (16.10 mg, 40.99 μmol) in anhydrous DCM (5 mL) stirring at −78° C. under argon, anhydrous DIPEA (5 μL), was added dropwise. Separately, 3-hydroxypropionitrile (4.59 mg, 40.99 μmol) was dissolved anhydrous DCM (500 μL) with dry DIPEA (1 μL, 6.15 μmol); this solution was then added dropwise to solution containing (2) The reaction was allowed to stir at −78° C. to ambient temperature over 2 hours. The reaction was then concentrated to a translucent oiL which was then purified via reverse-phase HPLC (Ailgent G 1 361A 1260 Infinity) using a stepwise gradient (5-90-% Buffer B over 10 minutes, 90-100% Buffer B over 7 minutes, 100% Bufler B over 8 minutes, 100-5% Buffer B over 5 minutes: Buffer A: dH₂O with 0.1% TFA Buffer B: CH₃CN+0.1% TFA). Fractions containing the desired product were combined and lyophilized to a white powder.

STEP 3. A mixture of palladium on carbon (10 wt %, 1 equiv. by mass) in THF/MeOH (2:3 ratio) was flushed with two H₂-containing balloons for 1 h. Separately, the compound obtained from STEP 2 was dissolved in MeOH and added to the palladium-containing slurry and allowed to react at room temperature for 12 h. The reaction was then filtered and concentrated under reduced pressure to a yellow oil. Then, the crude product was purified via reverse-phase HPLC (Aligent G 1361A J 260 Infinity) using a stepwise gradient (5-90% Buffer B over 10 minutes, 90-100% Buffer B over 7 minutes, 100% Buffer B over 8 minutes, 100-5% Buffer B over 5 minutes; Buffer A: dH₂O with 0.1% TFA, Buffer B: CH₃CN+0.1% TFA). Product-containing fractions were combined and lyophilized to a pale-yellow oil.

The following compounds were prepared using procedures similar to those above.

(((1-hydroxy-2-oxopiperidin-3-yl){(pyridin-2-ylmethyl)amino)phosphoryl)oxy)methyl pivalate (KY9). Analysis by ESI+(Expected [M+H]⁺=400.38. Observed [M+H]⁺=400.48).

((((3-fluorobenzyl)amino)(1-hydroxy-2-oxopiperidin-3-yl)phosphoryl)oxy)methyl pivalate (CDP12). Analysis by ESI+ Expected [M+H]⁺=400.38. Observed [M+H]⁺=400.44).

((((3,4-difluornhenzyl)amino)(1-hydro:xy-2-oxopiperidin-3-yl)phosphoryl)oxy)methyl pivalate (CDP13). Analysis by ESI+(Expected [M+H]⁺=400.38. Observed [M+H]⁺=400.44).

((((2,4-difluorobenzyi)amino)(1-hydrnxy-2-oxopiperidin-3-yl)phosphoryl)oxy)methyl pivalate (CDP14). Analysis by ESI+(Expected [M+H]⁺=400.38. Observed [M+H]⁺=400.44).

((((Cyclohexylmethyl)amino)(1-hydro:xy-2-oxopiperidin-3-yl)phosphoryl)oxy)methyl pivalate (CDP18). Analysis by ESI+(Expected [M+H]⁺=400.38. Observed [M+H]⁺=400.44).

((((2,6-difluorobenzyl)amino)(1-hydroxy-2-oxopiperidin-3-yl)phosphoryl)oxy)methyl pivalate (CDP19). Analysis by ESI+(Expected [M+H]⁺=400.38. Observed [M+H]⁺=400.44).

((((4-fluorobenzyl)amino)(1-hydroxy-2-oxopiperidin-3-yl)phosphoryl)oxy)methyl pivalate (VCY19). Analysis by ESI+(Expected [M+H]⁺=417.39. Observed [M+H]⁺=417.44).

((((Cyclopropylmethyl)amino)(1-hydroxy-2-oxopiperidin-3-yl)phosphoryl)oxy)methyl pivalate (VCY32). Analysis by ESI+(Expected [M+H]⁺=363.36. Observed [M+H]⁺=363.39).

(((Cyclobutylamino)(1-hydroxy-2-oxopiperidin-3-yl)phosphoryl)oxy)methyl pivalate (VCY34). Analysis by ESI+(Expected [M+H]⁺=363.36. Observed [M+H]⁺=363.98).

2-oxo-3-((2-(pivaloylthio)ethoxy)((pyridin-2-ylmethyl)amino)phosphoryl)piperidin-1-yl acetate (VCY31). Analysis by ESI+(Expected [M+H]⁺=472.51. Observed [M+H]⁺=472.44).

3-(((4-fluorobenzyl)amino)((5-nitrofuran-2-yl)methoxy)phosphoryl)-2-oxopiperidin-1-yl acetate (VCY22). ³¹PNMR (121 MHz, CDCl₃) 29.48 (d, J=17.25 Hz, 1P) ¹⁹FNMR (470 MHz, CDCl₃) 115.25 (m, 1F)

(((dimethylamino)(1-hydroxy-2-oxopiperidin-3-yl)phosphoryl)oxy)methyl pivalate (CDP21). Analysis by ESI+(Expected [M+H]⁺=337.15 Observed [M+Na]⁺=358.95). ¹H NMR (300 MHz, CDCl₃) δ 5.54-5.64 (m, 2H), 3.56-3.71 (m, 2H), 2.99-3.09 (m, 1H), 2.71-2.74 (d, J=9.0, 6H), 1.83-2.37 (m, 2H), 2.00-2.20 (m, 2H), 1.62-1.74 (m, 6H). ¹³C NMR (75 MHz, CDCl₃) 177.15 (s, 1C), 160.12 (d, J=1.19 Hz, 1C), 80.05-81.07 (d, J=6.0 Hz, 1C), 50.79 (s, 1C), 38.17-39.84 (d, J=123 Hz, 1C), 38.80 (s, 1C), 36.39-36.47 (d, J=5.25 Hz, 2C), 26.90 (s, 3C), 22.49 (d, J=2.72 Hz, 1C), 21.39 (d, J=2.72 Hz, 1C). ³¹P NMR (121 MHz, CDCl₃) δ 30.89 (1P), 29.04 (1P) (isomers).

N-dodecyl-P-(1-hydroxy-2-oxopiperidin-3-yl)phosphonamidic acid (CDP22). Analysis by ESI+(Expected [M+H]⁺=363.24. Observed [M+H]⁺=362.98). ¹H NMR (300 MHz, CDCl₃) 3.55-3.63 (m, 2H), 2.85-2.93 (m, 4H), 2.12-2.17 (m, 1H), 1.96-2.03 (m, 2H), 1.76-1.81 (m, 2H), 1.56-1.66 (m, 2H), 1.41-1.51 (m, 2H), 1.18 (m, 36H), 0.79-0.83 (t, J=6.0 Hz). ¹³C NMR (75 MHz, CDCl₃) 167.23 (s, 1C), 50.66 (s, 1C), 42.54 (s, 1C), 39.97 (s, 1C), 32.88 (d, J=7.5 Hz, 1C), 31.93 (s, 2C), 29.25-29.70 (m, 12C), 29.02 (s, 1C), 27.74 (s, 2C), 26.78 (s, 2C), 23.44 (s, 1C), 22.70 (s, 2C), 14.12 (s, 2C). ³¹P NMR (121 MHz, CDCl₃) 31.14 (1P), 22.54 (1P) (isomers).

(((1-hydroxy-2-oxopiperidin-3-yl)(methylamino)phosphoryl)oxy)methyl pivalate (CDP23). Analysis by ESI+(Expected [M+H]⁺=323.14 Observed [M+Na]⁺=344.91). ¹H NMR (300 MHz, CDCl₃) δ 5.60-5.69 (m, 2H), 3.86 (brs, 1H), 3.66-3.70 (m, 2H), 2.92-3.03 (dt, J=6.0 Hz, J=16.8 Hz, 1H), 2.71-2.74 (dd, J=5.4 Hz, J=12.3 Hz, 3H), 2.08-2.25 (m, 2H), 1.87-1.96 (m, 1H), 1.5 (s, 9H). ¹³C NMR (75 MHz, CDCl₃) 177.35 (s, 1C), 160.62 (d, J=1.20 Hz, C), 80.05-80.87 (d, J=6.20 Hz, 1C), 50.59 (s, 1C), 38.21-39.84 (d, J=121 Hz, 2C), 38.84 (s, 1C), 36.35-36.43 (d, J=5.2 Hz, 1C), 26.75 (s, 3C), 22.45 (d, J=2.75 Hz, 1C), 21.56 (d, J=2.75 Hz, HC). ³¹P NMR (121 MHz, CDC₃) δ 29.93 (HP) (isomers).

Additional NMR spectroscopic data are presented below. The solvent is CDCl₃ unless otherwise indicated.

| | | | ³¹P (121 MHz)/ |
|---|---|---|---|
| Cpd. | ¹H (300 MHz) | ¹³C(75 MHz) | ¹⁹F (282 MHz) |
| KY9 | δ 8.74 (d, J = 4.48 Hz, | δ 160.89 (d, J = 4.03 Hz, 1C), | ³¹P: δ 26.16 (1P), |
| | 1H), 8.17-8.22 (t, J = 7.93 | 123.50 (d, J = 3.68 Hz, 1C), 81.67 | 26.59 (1P). |
| | Hz, 8.37 Hz, 1H), 7.83- | (d, J = 7.61 Hz, 1C), 40.96 (s, 1C), | |
| | 7.85 (m, 1H), 7.62-7.67 | 26.84 (s, 1C), 22.07 (d, J = 5.68 | |
| | (t, J = 6.53 Hz, 6.94 Hz, | Hz, 1C), 21.67 (d, J = 7.44 Hz, 1C). | |
| | 1H), 5.62-5.64 (d, J = 7.04 | | |
| | Hz, 2H), 4.61-4.71 (m, | | |
| | 2H), 3.64 (s, 2H), 3.14- | | |
| | 3.27 (dt, J = 25.08 Hz, | | |
| | 1H), 1.99-2.19 (m, 2H), | | |
| | 1.78-1.91 (m, 2H), 1.18 | | |
| | (s, 9H). | | |
| VCY19 | 6.95-7.02 (m, 4H), 5.52 | 177.40 (s, 1C), 163.91 (d, J = 1.43 | ³¹P: δ 27.23 (1P), |
| | (s, 2H), 4.19-4.27 (m, | Hz, 1C), 135.96 (dd, J = 3.21 Hz, | 26.08 (1P). |
| | 2H), 3.58-3.63 (m, 2H), | 3.21, 5.14 Hz, 1C), 129.22-129.32 | ¹⁹F: −116.33 (s, |
| | 2.84-3.08 (m, 1H), 1.95- | (d, J = 8.16 Hz, 2C), 115.52 (d, | 1F), −116.27 (s, |
| | 2.25 (m, 3H), 1.78-1.89 | J = 3.14, 2C), 81.50 (d, J = 5.96 Hz, | 1F). |
| | (m, 1H), 1.16 (s, 9H). | 1C), 49.53 (s, 1C), 43.91 (s, 1C), | |
| | | 41.03-42.72 (d, J = 128.22 Hz, 1C), | |
| | | 27.03 (s, 3C), 21.51-22.15 (m, | |
| | | 2C). | |
| CDP24 | (MeOD) 7.02-7.46 (m, | (MeOD) 176.97 (s, 1C), 162.55 (s, | ³¹P (MeOD) δ |
| | 1H), 5.51-5.64 (m, 2H), | 1C), 160.11 (s, 1C), 129.66 (d, | 30.04 (1P), 29.13 |
| | 4.24-4.40 (m, 2H), 3.60- | J = 4.50 Hz, 1C), 129.33 (d, J = 8.25 | (1P). |
| | 3.67 (m, 2H), 2.88-3.15 | Hz, 1C), 126.94 (d, J = 5.25 Hz, | ¹⁹F: (MeOD) δ- |
| | (m, 1H), 2.16-2.29 (m, | 1C), 124.34 (d, J = 3.75 Hz, 1C), | 118.85 (s, 1F). |
| | 1H), 1.91-2.15 (m, 2H), | 115.55 (s, 1C), 81.06 (s, 1C), 48.67 | |
| | 1.81-1.91 (m, 1H), 1.21 | (s, 1C), 43.53 (s, 1C), 41.71-42.43 | |
| | (s, 9H). | (d, J = 54.00 Hz, 1C), 38.69 (s, 1C), | |
| | | 26.80 (s, 3C), 21.71 (m, 2C). | |
| CDP12 | (MeOD) 7.20-7.27 (m, | (MeOD) 176.89 (s, 1C), 164.66 (s, | ³¹P: (MeOD) δ |
| | 1H), 7.05-7.11 (m, 2H), | 1C), 162.23 (d, J = 1.43 Hz, 1C), | 31.18 (1P), 30.91 |
| | 6.95-6.85 (t, J = 8.29 Hz, | 143.39 (d, J = 3.21 Hz, 3.21 Hz, | (1P). |
| | 8.90 Hz, 1H), 5.56 (m, | 5.14 Hz, 1C), 129.82-129.93 (d, | ¹⁹F: (MeOD) δ- |
| | 2H), 4.09-4.16 (t, | J = 8.16 Hz, 2C), 113.59 (d, J = 5.25 | 114.08 (s, 1F), |
| | J = 10.08 Hz, 10.08 Hz, | Hz, 2C), 81.37 (d, J = 7.5 Hz, 1C), | −114.19 (s, 1F). |
| | 2H), 3.49-3.53 (t, J = 5.76, | 50.95 (s, 1C), 43.53 (s, 1C), 41.88- | |
| | 5.78 Hz, 2H), 3.02-3.16 | 43.16 (d, J = 96 Hz, 1C), 25.84 (s, | |
| | (dt, J = 25.13 Hz, 1H), | 3C), 21.51-22.03 (m, 2C). | |
| | 1.99-2.11 (m, 2H), 1.86- | | |
| | 1.97 (m, 1H), 1.73-1.84 | | |
| | (m, 1H), 1.10 (s, 9H). | | |
| CDP13 | 7.21 (m, 1H),7.11 (m, | δ 176.98 (s, 1C), 159.87 (d, | ³¹P: δ 29.95 (1P), |
| | 2H), 6.63 (bs, 1H), 5.60 | J = 1.92 Hz, 2C), 151.46 (s, 1C), | 28.79 (1P). |
| | (m, 2H), 4.28 (m, 2H), | 123.15 (s, 1C), 117.47 (d, J = 17.00 | ¹⁹F: δ −137.78 (d, |
| | 3.61-3.64 (t, J = 3.14 Hz, | Hz, 1C), 116.20 (d, J = 18.00 Hz, | J = 20.79 Hz, 1F), |
| | 4.17 Hz, 2H), 2.97-3.09 | 1C), 81.06 (d, J = 7.50 Hz, 1C), | −137.83 (d, J = 21.62 |
| | (dt, J = 23.84 Hz, 1H), | 48.56 (s, 1C), 38.76-40.54 (d, | Hz, 1F), −140.40 |
| | 2.19-2.28 (m, 1H), 1.99- | J = 134.60 Hz, 1C), 32.15-32.25 (t, | (d, J = 21.17 Hz, |
| | 2.12 (m, 2H), 1.80-1.92 | J = 3.75 Hz, 4.04 Hz, 1C), 26.78 (s, | 1F), 140.43 (d, |
| | (m, 1H), 1.18 (s, 9H). | 3C), 21.71 (d, J = 12.00 Hz, 1C), | J = 21.33 Hz, 1F). |
| | | 21.40 (d, J = 4.56 Hz, 1C). (a) | |
| CDP14 | 7.42 (m, 1H), 6.86 (m, | δ 177.11 (s, 1C), 159.65 (d, | ³¹P: δ 29.94 (1P), |
| | 2H), 6.12 (bs, 1H), 5.56- | J = 1.92 Hz, 1C), 130.49 (m, 1C), | 28.79 (1P). |
| | 5.60 (dd, J = 12.77 Hz, | 111.65 (s, 2C), 80.96 (d, J = 7.5 | ¹⁹F: δ −111.55 (d, |
| | 2H), 4.33 (m, 2H), 3.63 | Hz, 1C), 49.25 (s, 1C), 40.70- | J = 7.29 Hz, 1F), |
| | (m, 2H), 2.96-3.09 (dt, | 42.41 (d, J = 128.25 Hz, 1C), 38.08 | −111.66 (d, J = 7.41 |
| | J = 22.80 Hz, 1H), 2.17- | (t, J = 3.75 Hz, 1C), 26.81 (s, 3C), | Hz, 1F), −115.2 (d, |
| | 2.26 (m, 1H), 1.98-2.10 | 21.35-21.71 (m, 2C). (a) | J = 7.74 Hz, 1F), |
| | (m, 2H), 1.79-1.90 (m, | | −115.18 (d, J = 7.77 |
| | 1H), 1.17 (s, 9H). | | Hz, 1F). |
| CDP19 | δ 7.23 (m, 2H), 6.85-6.90 | δ 176.98 (s, 1C), 159.61 (d, | ³¹P: δ 29.28 (1P), |
| | (t, J = 7.51 Hz, 8.09 Hz, | J = 1.92 Hz, 2C), 129.58 (s, 1C), | 28.61 (1P). |
| | 1H), 5.51-5.57 (dd, | 111.65 (s,2C), 81.03 (d, J = 6.44 | ¹⁹F NMR −115.33 |
| | J = 13.24 Hz, 2H), 4.40 | Hz, 1C), 48.43 (s, 1C), 38.76- | (s, 2F). |
| | (m, 2H), 3.63 (m, 2H), | 40.54 (d, J = 134.60 Hz, 1C), | |
| | 2.99-3.10 (dt, J = 23.86 | 32.15-32.25 (t, J = 3.75 Hz, 4.04 | |
| | Hz, 1H), 2.17-2.26 (m, | Hz, 1C), 26.78 (s, 3C), 21.71 (d, | |
| | 1H), 1.97-2.11 (m, 2H), | J = 12.00 Hz, 1C), 21.40 (d, J = 4.56 | |
| | 1.80-1.90 (m, 1H), 1.17 | Hz, 1C). (a) | |
| | (s, 9H). | | |
| CDP18 | δ 5.54-5.64 (m, 2H), | 177.18 (s, 1C), 160.29 (d, J = 1.19 | ³¹P: δ 30.65 (1P), |
| | 3.61-3.65 (m, 2H), 2.88- | Hz, 1C), 92.29 (s, 1C), 81.35- | 29.94 (1P). |
| | 2.97 (m, 1H), 2.15-2.25 | 81.44 (d, J = 7.52 Hz, 1C), 40.62- | |
| | (m, 1H), 1.97-2.12 (m, | 42.34 (d, J = 130.41 Hz, 1C), 30.67 | |
| | 2H), 1.83-1.93 (m, 1H), | (d, J = 1.28 Hz, 1C) 27.00 (s, 3C), | |
| | 1.62-1.74 (m, 6H), 1.32- | 26.00 (s, 2C), 25.98 (s, 1C), 25.96 | |
| | 1.44 (m, 1H), 1.19 (s, | (s, 1C),21.85-21.89 (d, J = 2.72 | |
| | 9H), 0.79-0.94 (m, 3H). | Hz, 1C), 21.67-21.73 (d, J = 5.09 | |
| | | Hz, 1C). | |
| VCY32 | δ 5.54-5.59 (m, 2H), 3.59 | δ 177.12 (s, 1C), 160.77 (d, | ³¹P: δ 30.15 (1P), |
| | (m, 2H), 2.92-2.98 (dt, | J = 5.46 Hz, 1C), 81.23 (d, J = 2.76 | 28.65 (1P). |
| | J = 18.06 Hz, 1H), 2.78- | Hz, 1C), 49.98 (s, 1C), 45.87 (d, | |
| | 2.82 (q, J = 6.81 Hz, 7.07 | J = 0.95 Hz, 1C), 42.36-41.35 (d, | |
| | Hz, 7.07 MHz, 1H), | J = 127.79 Hz, 1C), 38.87 (s, 1C), | |
| | 2.14-2.20 (m, 1H), 2.07- | 27.03 (s, 3C), 22.03 (d, J = 4.83 | |
| | 2.13 (m, 2H), 1.77-1.85 | Hz, 1C), 21.64 (d, J = 4.83 Hz, 1C), | |
| | (m, 1H), 1.16 (s, 9H), | 13.38 (d, J = 6.46 Hz, 1C), 3.62 (s, | |
| | 0.44 (m, 1H), 0.17 (m, | 1C), 3.60 (s, 1C). | |
| | 1H). | | |
| VCY33 | (D₂O) 5 3.38-3.51 (m, | (D₂O) δ 163.18 (s, 1C), 52.38 (s, | ³¹P: (D₂O) δ 24.26 |
| | 2H), 2.49-2.74 (m, 3H), | 1C), 46.47 (s, 1C), 40.94-42.40 (d, | (1P). |
| | 1.95-2.05 (m, 1H), 1.82- | J = 34.5 Hz, 1C), 21.06 (s, 1C), | |
| | 1.92 (m, 2H), 1.66-1.75 | 17.01 (s, 1C), 11.99-12.11 (d, | |
| | (m, 1H), 0.83-0.94 (m, | J = 10.5 Hz, 1C), 2.71 (d, J = 2.25 | |
| | 1H), 0.37-0.40 (m, 2H), | Hz, 2C). | |
| | 0.04-0.09 (m, 2H). | | |
| VCY34 | δ 5.55-5.57 (d, J = 5.32 | δ 179.81 (s, 1C), 177.09-177.19 | ³¹P: δ 28.31 (1P), |
| | Hz, 2H), 3.78-3.91 (m, | (d, J = 7.21 Hz, 1C), 81.20-81.30 | 27.47 (1P). |
| | 1H), 3.57 (t, J = 4.76 Hz, | (d, J = 7.85 Hz, 1C), 50.15 (s, 1C), | |
| | 6.89 Hz, 2H), 2.90-2.98 | 46.56 (d, J = 3.64 Hz, 1C), 41.19- | |
| | (dt, J = 14.04 Hz, 1H), | 41.88 (d, J = 128.81 Hz, 1C), 38.79 | |
| | 2.26 (m, 2H), 2.10 (m, | (s, 2C), 26.92 (s, 1C), 22.02 (s, | |
| | 2H), 1.99 (m, 2H), 1.89 | 1C), 21.94 (d, J = 4.50 Hz, 1C), | |
| | (m, 2H), 1.60 (m, 2H), | 21.69 (d, J = 5.22 Hz, 1C). | |
| | 1.15 (s, 9H). | | |
| CDP25 | δ 5.61-5.74 (m, 2H), | 177.00 (s, 1C), 173.76 (d, J = 3.25 | ³¹P: δ 29.06 (1P). |
| | 5.00-5.07 (m, 2H), 4.01- | Hz, 1C), 160.40 (s, 1C), 116.93 (s, | |
| | 4.67 (m, 1H), 3.65-3.69 | 1C), 113.13 (s, 1C), 81.50 (d, | |
| | (m, 2H), 2.97-3.35 (m, | J = 7.50 Hz, 1C), 69.25 (s, 1C), | |
| | 1H), 2.20-2.29 (m, 1H), | 50.29 (s, 1C), 49.68 (s, 1C), 42.97 | |
| | 1.97-2.82 (m, 2H), 1.83- | (s, 1C), 41.22 (s, 1C), 38.75 (s, | |
| | 1.97 (m, 2H), 1.42-1.49 | 1C), 26.89 (s, 3C), 21.66 (s, 1C). | |
| | (m, 1H), 1.22-1.29 (m, | | |
| | 1H). | | |

(a) Some quaternary carbons were not observed.

### ADDITIONAL EXAMPLES

The following compounds were prepared using the procedures described below.

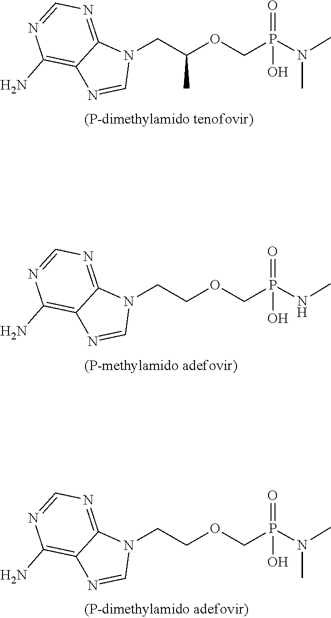 [SMILES: C[C@@H](Cn1cnc2c(N)ncnc21)OCP(=O)(O)N(C)C ; CNP(=O)(O)COCCn1cnc2c(N)ncnc21 ; CN(C)P(=O)(O)COCCn1cnc2c(N)ncnc21]

Synthesis of P—((((S)-1-(6-amino-9H-purin-9-yl)propan-2-yl)oxy)methyl)-N,N-dimethylphosphonamidic acid (P-dimethylamido tenofovir) (17). To a solution of triphenylphosphine (73.03 mg, 278.54 μmol) in anhydrous CHCl₃ (0.5 mL), diisopropyl (E)-diazene-1,2-dicarboxylate (54.16 μL, 278.54 μmol) was added at −78° C. under argon. The mixture was allowed to stir for 30 minutes. Separately, a solution of tenofovir ((S)-(((1-(6-amino-9H-purin-9-yl)propan-2-yl)oxy)dimethyl)phosphonic acid, 10 mg, 34.82 μmol), dimethylamine-HCl (22.71 mg, 278.54 μmol) and DIPEA (20.04 μL, 114.90 μmol) was prepared in anhydrous CHCl₃ (0.5 mL); this was then added dropwise to the solution containing the betaine at −78° C. The reaction stirred under argon for 30 minutes. The crude reaction mixture was then transferred to a 50 mL Falcon tube, where 1 volume of water was added. The reaction was vortexed and centrifuged (4000 rpm, 4° C.) for 2 minutes. The aqueous layer was then isolated and lyophilized to a colorless gum. The crude product was purified via reverse-phase HPLC (Agilent G1361A 1260 Infinity) using a stepwise gradient (CH₃CN:H₂O 1:99-40:60, 30 min). The product (17) was used without further purification. Analysis by ESI− (Expected [M−H]⁻=313.28. Observed [M−H]⁻=313.16). ¹H NMR (300 MHz, D₂O) δ 8.09 (s, 1H), 8.05 (s, 1H), 4.21 (m, 2H), 3.90 (m, 1H), 3.33-3.55 (m, 2H), 2.16 (dd, J=9.60 Hz, 6H), 1.12 (d, J=6.30 Hz, 3H). ¹³C NMR (75 MHz, D₂O) δ 155.39 (s, 1C), 152.54 (s, 1C), 149.09 (s, 1C), 143.46 (s, 1C), 118.04 (s, 1C), 75.72 (s, 1C), 62.75 (s, 1C), 48.17 (s, 1C), 35.85 (s, 1C), 15.97 (s, 1C). ³¹P NMR (121 MHz, D₂O) δ 21.90

Synthesis of P-((2-(6-amino-9H-purin-9-yl)ethoxy)methyl)-N-methylphosphonamidic acid (P-methylamido adefovir) (16). To a solution of triphenylphosphine (153.62 mg, 585.68 μmol) in anhydrous NMP (1.5 mL), diisopropyl (E)-diazene-1,2-dicarboxylate (113.87 μL, 585.68 μmol) was added at −78° C. under argon. The mixture was allowed to stir for 30 minutes. Separately, adefovir ((2-(6-amino-9H-purin-9-yl)ethoxy)methyl)phosphonic acid, 20 mg, 73.21 μmol) was dissolved in 250 μL DMSO, to which was added methylamine (2.0 M in THF, 292.84 μL, 585.68 μmol) and DBU (87.42 μL, 585.68 μmol) in NMP (1.5 mL); this was then added dropwise to the solution containing the betaine at −78° C. The reaction stirred under argon for 30 minutes. The crude reaction mixture was then transferred to a 50 mL Falcon tube, where 1 volume of water was added. The reaction was vortexed and centrifuged (4000 rpm, 4° C.) for 2 minutes. The aqueous layer was then isolated and lyophilized to a colorless gum. The crude product was purified via reverse-phase HPLC (Agilent G1361A 1260 Infinity) using a stepwise gradient (CH₃CN:H₂O 1:99-40:60, 30 min). The product (16) was used without further purification. Analysis by ESI− (Expected [M−H]⁻=286.23. Observed [M−H]⁻=286.35). ¹H NMR (300 MHz, MeOD) δ 8.26 (s, 1H), 8.18 (s, 1H), 4.40 (t, J=3.00 Hz, 2H), 3.86 (t, J=4.50 Hz, 2H), 3.58 (d, J=8.40 Hz, 2H), 2.45 (d, J=11.10 Hz, 3H). ¹³C NMR (75 MHz, MeOD) δ 157.20 (s, 1C), 153.55 (s, 1C), 150.55 (s, 1C), 143.88 (s, 1C), 119.79 (s, 1C), 71.83 (s, 1C), 70.00 (s, 1C), 68.11 (s, 1C), 25.77 (s, 1C). ³¹P NMR (121 MHz, MeOD) δ 21.03.

Synthesis of P-((2-(6-amino-9H-purin-9-yl)ethoxy)methyl)-N,N-dimethylphosphonamidic acid (P-dimethylamido adefovir) (18). To a solution of triphenylphosphine (38.40 mg, 146.42 μmol) in anhydrous CHCl₃ (0.25 mL), diisopropyl (E)-diazene-1,2-dicarboxylate (28.47 μL, 146.42 μmol) was added at −78° C. under argon. The mixture was allowed to stir for 30 minutes. Separately, adefovir ((2-(6-amino-9H-purin-9-yl)ethoxy)methyl)phosphonic acid, 5 mg, 18.30 μmol) was dissolved in NMP (0.25 ml), to which was added dimethylamine-HCl (11.94 mg, 146.42 μmol) and DBU (21.85 μL, 146.42 μmol) in CHCl₃ (0.25 mL); this was then added dropwise to the solution containing the betaine at −78° C. The reaction stirred under argon for 30 minutes. The crude reaction mixture was then transferred to a 50 mL Falcon tube, where 1 volume of water was added. The reaction was vortexed and centrifuged (4000 rpm, 4° C.) for 2 minutes. The aqueous layer was then isolated and lyophilized to a colorless gum. The crude product was purified via reverse-phase HPLC (Agilent G1361A 1260 Infinity) using a stepwise gradient (CH₃CN:H₂O 1:99-40:60, 30 min). The product (18) was used without further purification. Analysis by ESI− (Expected [M−H]⁻=299.23. Observed [M−H]⁻=299.13). ¹H NMR (300 MHz, D₂O) δ 8.15 (s, 1H), 8.12 (s, 1H), 4.35 (t, J=4.80 Hz, 2H), 3.81 (t, J=4.50 Hz, 2H), 3.50 (m, 2H), 2.20 (d, J=11.10 Hz, 3H). ¹³C NMR (75 MHz, D₂O) δ 155.47 (s, 1C), 152.31 (s, 1C), 148.83 (s, 1C), 143.00 (s, 1C), 118.20 (s, 1C), 70.85 (s, 1C), 67.24 (s, 1C), 65.38 (s, 1C), 36.06 (s, 1C). ³¹P NMR (121 MHz, D₂O) δ 20.84

### Biological Assays

### Crystal Violet Cell Proliferation Assay Against D423 Cells

D423 cells were incubated with CDP21 or CDP23 for 5 days. See, Leonard et al. (2016) Nat. Chem. Biol. 12:1053-1058 and Satani et al. (2016) PLos One, 11:e0168739, each of which is incorporated by reference for all purposes. Then, cells were fixed and stained with crystal violet and quantified spectroscopically. Cell density as measured by crystal violet were plotted as a function of inhibitor. While both compounds are selective for ENO1-deleted cells, CDP23 exhibits a more than 2.5-fold greater potency compared to CDP21 (IC₅₀=40 nM versus 108 nM).

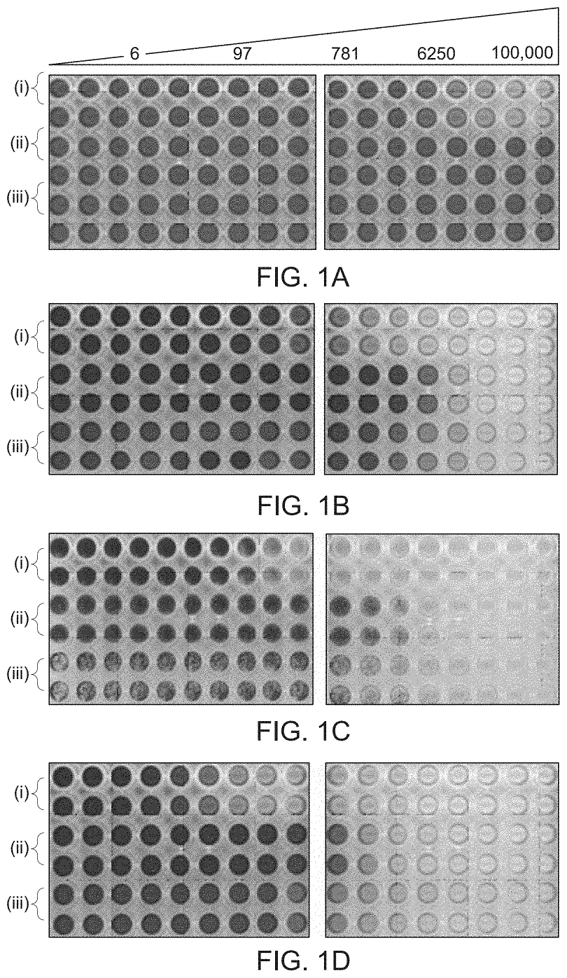

FIGS. 1A, 1B, 1C, and 1D show representative cell proliferation assays for the compounds HEX (1A), FLM37 (1B), CDP18 (1C), and VCY32 (1D). For each compound, assays are run against (i) ENO1-deleted (D423), (ii) ENO1-rescued (D423 ENO1), and (iii) ENO1-wildtype (LN319) glioma cells. Whereas HEX displays very little activity against any of the three cells, FLM37, CDP18, and VCY32 all show activity at the micromolar range or better. The improved behavior of FLM37, CDP18, and VCY32 can be attributed to their increased hydrophilicity.

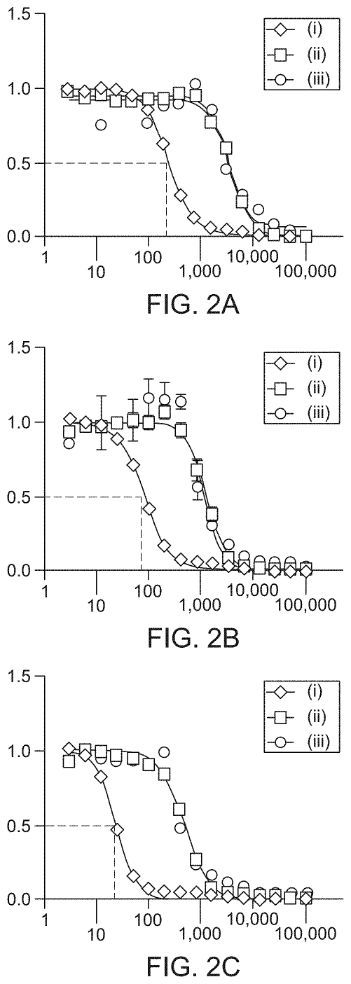

FIGS. 2A, 2B, and 2C present quantitative results from the above cell proliferation assays. IC₅₀ values, represented by the dashed lines, are in all cases less than 10 M. Particularly noteworthy is the sub-micromolar inhibition of enolase in ENO1-deleted (D423) glioma cells by all three compounds.

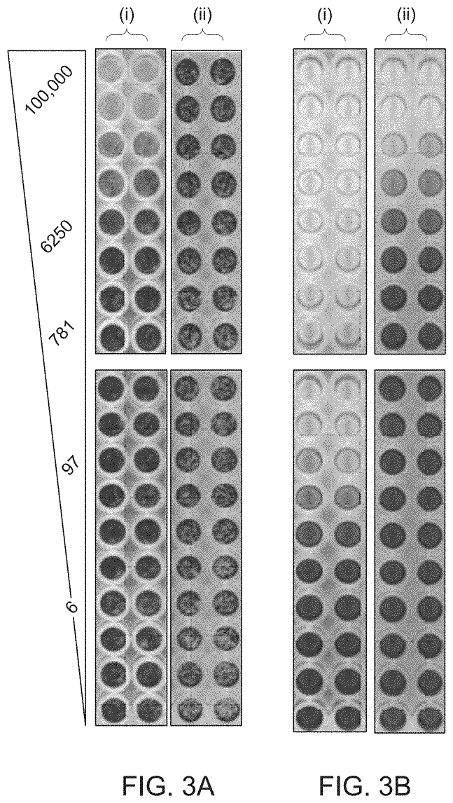

FIGS. 3A and 3B show the dramatic effect of lipophilicity on enolase inhibition in the cell proliferation assay. Cyclopropyl compound VCY33 (3A), having a log P of 4.04, shows inhibition of ENO1-deleted (D423) cells (i) at concentrations of 1 μM or higher; no inhibition is readily apparent in ENO1-wildtype (LN319) glioma cells (ii). In contrast, CDP22, having a long-chain alkyl moiety and a log P of −0.57, inhibits ENO1-deleted (D423) cells at sub-micromolar levels, and shows activity in ENO1-wildtype (LN319) cells.

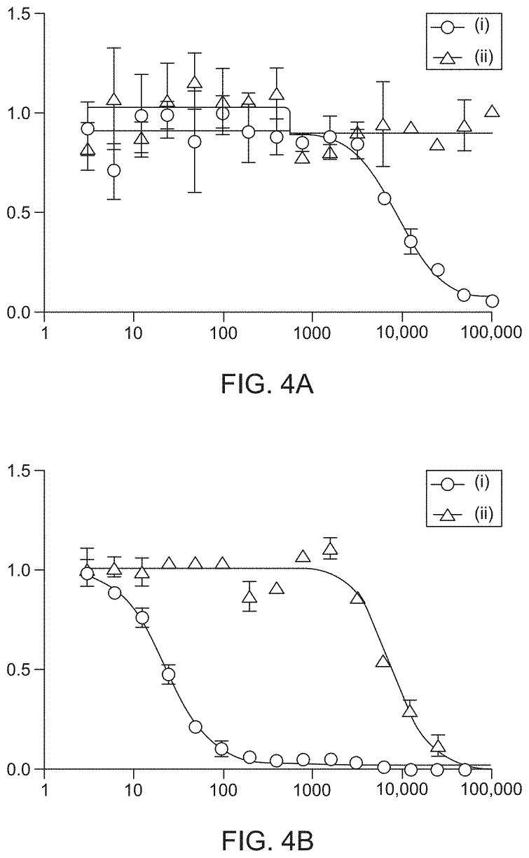

FIGS. 4A and 4B present quantitative results from the above cell proliferation assays. The enhanced activity due to increased lipophilicity is evident on comparing IC₅₀ values for ENO1-wildtype (LN319) glioma cells (i): 8828 nM for VCY33 (4A) vs. 23.97 nM for CDP22 (4B).

A comparison of IC₅₀ values (in nM) for ENO1-deleted (D423), ENO1-rescued (D423 ENO1), and ENO1-wildtype (LN319) glioma cells by CDP21, CDP22 and CDP23 is shown below.

| | | IC50,D423 | IC50,D423 ENO1 | IC50, LN319 |
|---|---|---|---|---|
| Entry | Compound | (ENO1−/−) | (ENO1+/+) | (ENO1+/+) |
| 11 | CDP21 | 108 | 2169 | 5063 |
| 12 | CDP22 | 58 | N/A | 844 |
| 13 | CDP23 | 40 | 790 | 1,500 |

IC₅₀ values (in nM) and log P values for compounds of Formula V are disclosed below, along with data for HEX and POMHEX for comparison.

| | | |
|---|---|---|
| 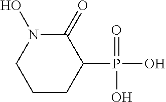 [SMILES: O=C1C(P(=O)(O)O)CCCN1O] | 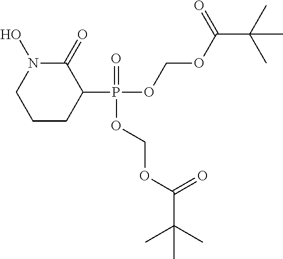 [SMILES: CC(C)(C)C(=O)OCOP(=O)(OCOC(=O)C(C)(C)C)C1CCCN(O)C1=O] | 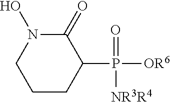 |
| HEX | POMHEX | (Formula V) |
| | | R⁶ = —CH₂OCOC(CCH₃)₃ |

| | | | IC50, | |
|---|---|---|---|---|
| | | | D423 | |
| | | | EN01 | |
| Entry | Compound | R³R⁴NH | (ENO1 +/+) | LogP |
| 1 | HEX | — | 7,574.0 | −1.52 |
| 2 | POMHEX | — | 17.3 | 2.13 |
| 3 | FLM37 | 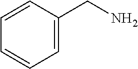 | 244.4 | 1.78 |
| 4 | KY9 | 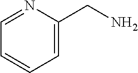 | 96.2 | 0.61 |
| 5 | CDP24 | 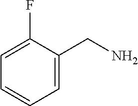 | 195.6 | 1.90 |
| 6 | CDP12 | 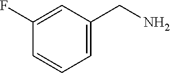 | 102.7 | 1.90 |
| 7 | VCY19 | 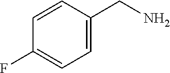 | 129.2 | 1.95 |
| 8 | CDP13 | 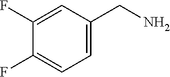 | 84.6 | 2.04 |
| 9 | CDP14 | 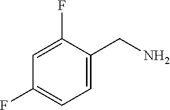 | 80.3 | 2.04 |
| 10 | CDP19 | 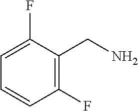 | 116.4 | 2.01 |
| 11 | CDP18 | 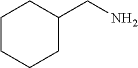 | 81.1 | 2.67 |
| 12 | VCY32 | 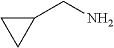 | 22.0* | 1.25 |
| 13 | VCY34 | 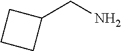 | 121.0* | 1.04 |
| 14 | CDP21 | 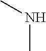 | 114.8 | 0.63 |
| 15 | CDP23 | 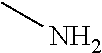 | 62.58 | 0.38 |
| 16 | CDP25 | 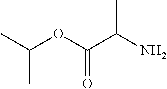 | 257.8 | 1.31 |

*IC50s in reference to respective POMHEX controls (VCY32 IC50 = 22 nM vs POMHEX IC50 = 41.47 nM; VCY34 IC50 = 121.0 nM vs POMHEX IC50 = 217.0 nM)

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for preparing a compound of Formula I 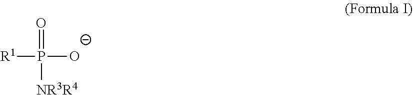
wherein
R¹ is chosen from —R² and —OR²;
R² is chosen from alkyl_{(C≤12)}, cycloalkyl_{(C≤12)}, heterocycloalkyl_{(C≤12)}, aryl_{(C≤12)}, and heteroaryl_{(C≤12)}, or a substituted version of any of these groups;
R³ is chosen from hydrogen, alkyl_{(C≤18)}, and substituted alkyl_{(C≤18)}; and
R⁴ is chosen from alkyl_{(C≤18)}, aryl_{(C≤18)}, aralkyl_{(C≤18)}, heteroaralkyl_{(C≤18)}, alkanediyl_{(C≤18)}-alkoxy_{(C≤18)}, cycloalkyl_{(C≤18)}, alkanediyl_{(C≤18)}-cycloalkyl_{(C≤18)}, or a substituted version of any of these groups; or
R³ and R⁴, together with the intervening nitrogen, combine to form a 5-7 membered heterocycloalkyl;
said method comprising:
treating a compound of Formula II 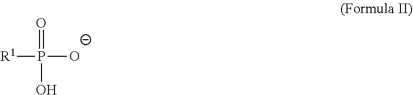
with a dialkylazodicarboxylate; a phosphine reagent; and an amine of Formula III
NHR³R⁴ (Formula III)
in an aprotic solvent to yield a compound of Formula I.

2. (canceled)

3. (canceled)

4. (canceled)

5. (canceled)

6. (canceled)

7. (canceled)

8. The method of claim 1, wherein the compound of Formula II is fludarabine monophosphate.

9. The method of claim 1, wherein R¹ is 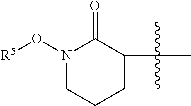
wherein R⁵ is hydrogen, alkyl_{(C≤12)}, aryl_{(C≤12)}, heteroaryl_{(C≤12)}, aralkyl_{(C≤12)}, heteroaralkyl_{(C≤12)}, or acyl_{(C≤12)}, or a substituted version of any of these groups.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. The method of claim 1, wherein the amine of Formula III is an amino acid.

44. The method of claim 1, wherein the dialkylazodicarboxylate is chosen from diisopropyl azodicarboxylate (DIAD), diethyl azodicarboxylate (DEAD), and di-2-methoxyethyl azodicarboxylate (DMEAD)

45. (canceled)

46. The method of claim 1, wherein the phosphine reagent is triphenylphosphine or tributylphosphine.

47. (canceled)

48. (canceled)

49. (canceled)

50. (canceled)

51. The method of claim 1, wherein the aprotic solvent is chosen from dichloromethane, chloroform, ethyl acetate, tetrahydrofuran, acetone, dimethylformamide, acetonitrile, and dimethylsulfoxide.

52. (canceled)

53. (canceled)

54. The method of claim 1, wherein the triphenylphosphine and the dialkylazodicarboxylate are combined to form a betaine prior to treatment with the compound of Formula II and/or the compound of Formula III.

55. A compound of Formula I prepared by the method of claim 1.

56. A compound chosen from
N-benzyl-P-(1-(benzyloxy)-2-oxopiperidin-3-yl)phosphonamidic acid;
P-(1-(benzyloxy)-2-oxopiperidin-3-yl)-N-(pyridin-2-ylmethyl)phosphonamidic acid;
P-(1-(benzyloxy)-2-oxopiperidin-3-yl)-N-(pyridin-3-ylmethyl)phosphonamidic acid;
P-(1-(benzyloxy)-2-oxopiperidin-3-yl)-N-(3-fluorobenzyl)phosphonamidic acid;
P-(1-(benzyloxy)-2-oxopiperidin-3-yl)-N-(4-fluorobenzyl)phosphonamidic acid;
P-(1-(benzyloxy)-2-oxopiperidin-3-yl)-N-(2,4-difluorobenzyl)phosphonamidic acid;
P-(1-(benzyloxy)-2-oxopiperidin-3-yl)-N-(3,4-difluorobenzyl)phosphonamidic acid;
P-(1-(benzyloxy)-2-oxopiperidin-3-yl)-N-methyl-N-(2-(pyrrolidin-1-yl)ethyl phosphoramidic acid;
P-(1-(benzyloxy)-2-oxopiperidin-3-yl)-N-(prop-2-yn-1-yl)phosphonamidic acid;
P-(1-(benzyloxy)-2-oxopiperidin-3-yl)-N-(4-fluorophenyl)phosphonamidic acid;
N-(2-(aminomethyl)phenyl)-P-(1-(benzyloxy)-2-oxopiperidin-3-yl)phosphonamidic acid;
P-(1-(benzyloxy)-2-oxopiperidin-3-yl)-N-(3-hydroxybenzyl)phosphonamidic acid;
naphthalen-1-yl hydrogen benzylphosphoramidate;
naphthalen-1-yl hydrogen (pyridin-2-ylmethyl)phosphoramidate;
naphthalen-1-yl hydrogen (4-fluorobenzyl)phosphoramidate;
naphthalen-1-yl hydrogen (2,6-difluorobenzyl)phosphoramidate;
naphthalen-1-yl hydrogen (cyclohexylmethyl)phosphoramidate;
naphthalen-1-yl hydrogen cyclopropylphosphoramidate;
isopropyl (hydroxy(naphthalen-1-yloxy)phosphoryl)-L-alaninate;
((2S,3R,4R,5R)-5-(6-amino-2-fluoro-9H-purin-9-yl)-3,4-dihydroxytetrahydrofuran-2-yl)methyl hydrogen benzylphosphoramidate;
((2S,3R,4R,5R)-5-(6-amino-2-fluoro-9H-purin-9-yl)-3,4-dihydroxytetrahydrofuran-2-yl)methyl hydrogen (pyridin-2-ylmethyl)phosphoramidate;
((2S,3R,4R,5R)-5-(6-amino-2-fluoro-9H-purin-9-yl)-3,4-dihydroxytetrahydrofuran-2-yl)methyl hydrogen cyclopropylphosphoramidate;
isopropyl ((((2S,3R,4R,5R)-5-(6-amino-2-fluoro-9H-purin-9-yl)-3,4-dihydroxytetrahydrofuran-2-yl)methoxy)(hydroxy)phosphoryl)-D-alaninate;
N-benzyl-P-(1-hydroxy-2-oxopiperidin-3-yl)phosphonamidic acid;
P-(1-acetoxy-2-oxopiperidin-3-yl)-N-benzylphosphonamidic acid;
N-dodecyl-P-(1-hydroxy-2-oxopiperidin-3-yl)phosphonamidic acid;
P—((((S)-1-(6-amino-9H-purin-9-yl)propan-2-yl)oxy)methyl)-N-methylphosphonamidic acid;
1-(6-amino-9H-purin-9-yl)propan-2-yl)oxy)methyl)-N-methylphosphonamidic acid;
P-((2-(6-amino-9H-purin-9-yl)ethoxy)methyl)-N-methylphosphonamidic acid; and
P-((2-(6-amino-9H-purin-9-yl)ethoxy)methyl)-N,N-dimethylphosphonamidic acid,
or a salt thereof.

57. The method of claim 1, further comprising converting a compound of Formula I to a compound of Formula IV 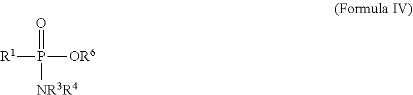
wherein
R⁶ is hydrogen, or
alkyl_{(C≤12)}, acyl_{(C≤12)}, aralkyl_{(C≤12)}, heteroaralkyl_{(C≤12)}, or a substituted version of any of these groups; or
-L₁-R⁷ wherein
L₁ is alkanediyl_{(C≤8)} or substituted alkanediyl_{(C≤8)}; and
R⁷ is acyl_{(C≤12)}, acyloxy_{(C≤12)}, acylthio_{(C≤12)}, —C(O)-alkoxy_{(C≤12)}, —OC(O)-heterocycloalkanediyl_{(C≤12)}-heterocycloalkyl_{(C≤12)}, or a substituted version of any of these groups, or
R⁷ and R⁴ are taken together and are -alkanediyl_{(C≤12)}-arenediyl_{(C≤12)}- or substituted alkanediyl_{(C≤12)}-arenediyl_{(C≤12)}.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A compound of Formula IV prepared by the method of claim 57.

78. A compound chosen from
3-((benzylamino)(2-(pivaloylthio)ethoxy)phosphoryl)-2-oxopiperidin-1-yl acetate;
3-((benzylamino)((S-nitrofuran-2-yl)methoxy)phosphoryl)-2-oxopiperidin-1-yl acetate;
3-((benzylamino)((5-nitrothiophen-2-yl)methoxy)phosphoryl)-2-oxopiperidin-1-yi acetate;
3-((benzylamino)((1-methyl-2-nitro-1H-imidazol-5-yl)methoxy)phosphoryl)-2-oxopiperidin-1-yl acetate;
(((1-hydroxy-2-oxopiperidin-3-yl){(pyridin-2-ylmethyl)amino)phosphoryl)oxy)methyl pivalate;
((((3-fluorobenzyl)amino)(1-hydroxy-2-oxopiperidin-3-yl)phosphoryl)oxy)methyl pivalate;
((((3,4-difluorobenzyl)amino)(1-hydroxy-2-oxopiperidin-3-yl)phosphoryl)oxy)methyl pivalate;
((((2,4-difluorobenzyl)amino)(1-hydroxy-2-oxopiperidin-3-yl)phosphoryl)oxy)methyl pivalate;
((((Cyclohexylmethyl)amino)(1-hydroxy-2-oxopiperidin-3-yl)phosphoryl)oxy)methyl pivalate;
((((2,6-difluorobenzyl)amino)(1-hydroxy-2-oxopiperidin-3-yl)phosphoryl)oxy)methyl pivalate;
((((4-fluorobenzyl)amino)(1-hydroxy-2-oxopiperidin-3-yl)phosphoryl)oxy)methyl pivalate;
((((Cyclopropylmethyl)amino)(1-hydroxy-2-oxopiperidin-3-yl)phosphoryl)oxy)methyl pivalate;
(((Cyclobutylamino)(1-hydroxy-2-oxopiperidin-3-yl)phosphoryl)oxy)methyl pivalate;
2-oxo-3-((2-(pivaloylthio)ethoxy)((pyridin-2-ylmethyl)amino)phosphoryl)piperidin-1-yl acetate;
3-(((4-fluorobenzyl)amino)((5-nitrofuran-2-yl)methoxy)phosphoryl)-2-oxopiperidin-1-yl acetate;
2-cyanoethyl N-benzyl-P-(1-hydroxy-2-oxopiperidin-3-yl)phosphonamidate;
(((benzylamino)(1-hydroxy-2-oxopiperidin-3-yl)phosphoryl)oxy)methyl [1,4′-bipiperidine]-1′-carboxylate;
(((dimethylamino)(1-hydroxy-2-oxopiperidin-3-yl)phosphoryl)oxy)methyl pivalate;
(((1-hydroxy-2-oxopiperidin-3-yl)(methylamino)phosphoryl)oxy)methyl pivalate;
((((2-fluorobenzyl)amino)(1-hydroxy-2-oxopiperidin-3-yl)phosphoryl)oxy)methyl pivalate; and
(((1-hydroxy-2-oxopiperidin-3-yl)((1-isopropoxy-1-oxopropan-2-yl)amino)phosphoryl)oxy)methyl pivalate, or a salt thereof.

79. The method of claim 1, further comprising combining the compound of Formula I with a pharmaceutically acceptable carrier to produce a pharmaceutical composition comprising a compound of Formula I.

80. A pharmaceutical composition comprising a compound of Formula I prepared by the method of claim 79.

81. (canceled)

82. (canceled)

83. (canceled)

84. (canceled)

85. (canceled)

86. A method of inhibition of an enolase-mediated disease in a patient in need thereof, the method comprising the administration of a therapeutically effective amount of a compound of claim 55, or a salt thereof, to the patient.

87. (canceled)

88. A method of inhibition of an RNA polymerase-mediated disease in a patient in need thereof, the method comprising the administration of a therapeutically effective amount of a compound of claim 55, or a salt thereof, to the patient.

89. A method of treating HIV infection in a patient in need thereof, comprising administering a compound of claim 55, or a salt thereof, optionally in combination with one or more other antiretroviral agents, to the patient.

90. (canceled)

91. (canceled)

92. (canceled)

93. (canceled)

94. A method of treating chronic hepatitis B in a patient in need thereof, comprising administering a compound of claim 55, or a salt thereof, to the patient.

95. (canceled)

96. A method of inhibition of a disease mediated by an enzyme chosen from RNA reductase and DNA polymerase in a patient in need thereof, the method comprising the administration of a therapeutically effective amount of a compound of claim 55, or a salt thereof, to the patient.

97. A method of treating B-cell chronic lymphocytic leukemia in a patient in need thereof, comprising administering a compound of claim 55, or a salt thereof, to the patient.
